SEQUENCE LISTING (22 sequences)

SEQ ID NO: 1 - DNA, 34 bp, Artificial Sequence
  cleavage site
ataacttcgtataatgtatgctatacgaagttat

SEQ ID NO: 2 - DNA, 35 bp, Artificial Sequence
  primer
cggaattccgcaggttttgtaatgtatgtgctcgt

SEQ ID NO: 3 - DNA, 38 bp, Artificial Sequence
  primer
ctccgaagcttaagcccgatatcgtctttcccgtatca

SEQ ID NO: 4 - DNA, 22 bp, Artificial Sequence
  probe
accccgtacgtcttcccgagcg

SEQ ID NO: 5 - DNA, 22 bp, Artificial Sequence
  primer
gggatctgccattgtcagacat

SEQ ID NO: 6 - DNA, 20 bp, Artificial Sequence
  primer
tggtgtgggccataattcaa

SEQ ID NO: 7 - DNA, 22 bp, Artificial Sequence
  probe
tgctggcaccagacttgccctc

SEQ ID NO: 8 - DNA, 20 bp, Artificial Sequence
  primer
cggctaccacatccaaggaa

SEQ ID NO: 9 - DNA, 18 bp, Artificial Sequence
  primer
gctggaattaccgcggct

SEQ ID NO: 10 - DNA, 13 bp, Homo sapiens
  misc_feature 3: n = t or g
canratatgarat

SEQ ID NO: 11 - DNA, 22 bp, Artificial Sequence
  probe
caggaccacttctgcgctcggc

SEQ ID NO: 12 - DNA, 23 bp, Artificial Sequence
  primer
cggctccagatttatcagccaat

SEQ ID NO: 13 - DNA, 47 bp, Artificial Sequence
  Flp recombinase target site (sense strand)
gaagttcctattccaagttcctattctctagaaagtataggaacttc

SEQ ID NO: 14 - DNA, 47 bp, Artificial Sequence
  Flp recombinase target site (antisense strand)
gaagttcctatactttctagagaataggaacttggaataggaacttc

SEQ ID NO: 15 - DNA, 22 bp, Mus musculus
actgtatcactttttcacaatt

SEQ ID NO: 16 - DNA, 19 bp, Mus musculus
actgtaccgatacaaaacc

SEQ ID NO: 17 - DNA, 23 bp, Mus musculus
actgtagttctgtagatatctgt

SEQ ID NO: 18 - DNA, 22 bp, Mus musculus
actgtacagacagcattgtaat

SEQ ID NO: 19 - DNA, 23 bp, Mus musculus
acatttgaaggctgaagtacagt

SEQ ID NO: 20 - DNA, 22 bp, Mus musculus
cagagaatcctaggggtacagt

SEQ ID NO: 21 - DNA, 24 bp, Mus musculus
ctcagcgaatttattcattacagt

SEQ ID NO: 22 - DNA, 24 bp, Mus musculus
ggtcctgaagttcaatcttacagt

(19) United States
(12) Patent Application Publication
(10) Pub. No.: US 2008/0226601 A1
(43) Pub. Date: Sep. 18, 2008

(54) Herpes Virus-Based Compositions and Methods of Use in the Prenatal and Perinatal Periods

(75) Inventors: Howard J. Federoff, Bethesda, MD (US); William J. Bowers, Webster, NY (US)

Correspondence Address: FISH & RICHARDSON P.C., P.O BOX 1022, Minneapolis, MN, 55440-1022, US

(73) Assignee: UNIVERSITY OF ROCHESTER, Rochester, NY (US)

(21) Appl. No.: 11/916,434

(22) PCT Filed: Jun. 5, 2006
(86) PCT No.: PCT/US06/21806

§ 371 Date: Mar. 28, 2008

Related U.S. Application Data
(60) Provisional application No. 60/687,356, filed on Jun. 3, 2005.

(51) Int. Cl. A61K 48/00; C12Q 1/70

(52) U.S. Cl. 424/93.2; 435/5

(57) ABSTRACT

Disclosed are compositions and methods for reducing the severity of a birth defect in a mammal by exposing the mammal (e.g., in utero) to a herpes virus amplicon particle comprising a cis element-flanked transgene and a sequence encoding a transposase. Upon expression, the transposase inserts the transgene into the genome of a cell (e.g., a neuron) within the mammal and the transgene expresses a polypeptide or RNA that compensates for a protein or gene defect that is causally associated with the birth defect.

### RELATED APPLICATIONS

This application claims the benefit of an earlier-filed provisional application, U.S. Ser. No. 60/687,356, filed Jun. 3, 2005, the content of which is hereby incorporated by reference in its entirety.

### STATEMENT REGARDING GOVERNMENT SUPPORT

The work described herein was funded, in part, by grants from the National Institutes of Health (U54-NS045309 and R01-NS364201). The United States government may, therefore, have certain rights in the invention.

### FIELD OF THE INVENTION

The present invention relates generally to herpes virus amplicon particles and herpes viruses in which artificial chromosomes have been packaged. These compositions can be used to screen for and administer therapeutic agents effective in treating medical disorders, including birth defects.

### SUMMARY

A significant obstacle to treating birth defects is the lack of safe and efficient vehicles that can be used to deliver nucleic acid sequences to a cell within the affected animal while in utero. We have discovered herpes virus-based compositions that can be used as such delivery vehicles. We have also designed altered herpes viruses that have packaged modified artificial chromosomes and that can be used to screen for therapeutic agents effective in reducing the severity of a birth defect. The altered herpes viruses, which are described further below, can also be used to identify a cellular target for therapeutic intervention during the prenatal or perinatal periods.

The herpes virus-based compositions can be used to achieve a more persistent expression of a therapeutic agent when modified to assume a chromosomally integrating form. Herpes virus-derived amplicons are vectors devoid of viral genes that normally exist episomally within transduced cells. Thus, they are replication-defective. We have combined the Tcl-like Sleeping Beauty (SB) transposon system with the amplicon to produce a vehicle that carries sequences that are subsequently integrated into the genome of a host cell. Our experiments demonstrate expression in many areas of the brain and prolonged transgene expression in neurons following in utero administration (e.g., infusion) of these vehicles. When cells contain both an enzyme that mediates chromosomal integration and a corresponding amplicon particle bearing a heterologous transgene (e.g., a sequence encoding an agent that reduces the severity of a birth defect), the transgene can integrate into the genomes of affected cells, regardless of whether those cells are mitotically active or post-mitotic. Methods of making herpes virus-based amplicon particles containing a transgene that, upon introduction into a host cell, integrates into the host cell's genome are described below, and vehicles made by such methods are within the scope of the present invention. The transgene can be flanked by cis elements and can encode or express a polypeptide or RNA that compensates for a protein or gene defect that is causally associated with a birth defect.

Unless it is clear from the context that a different meaning is intended, the terms “vehicle”, “vector”, and “particle” are used interchangeably.

The vectors of the invention are exemplified by (but not limited to) HSVT0-βgeo, which contains an SV40 promoter-driven β-galactosidase-neomycin (βgeo) fusion transgene flanked by the SB inverted/direct repeats, and HSVsb, which contains the SB transposase gene transcriptionally driven by the HSV immediate-early 4/5 gene promoter. Co-transduction of these two vectors into mitotically-active baby hamster kidney (BHK) cells resulted in integration, maintenance and expression of the transgenon. This bipartite amplicon platform was also used to successfully introduce the transgenon into primary murine cortical cultures. In addition, co-delivery of these vectors to the brains of E14.5 C57BL/6 mouse embryos resulted in the birth of viable neonates, integration of the transposable element from HSVT-βgeo, and an extended period of transgene expression (at least 90 days) when compared to embryos transduced with HSVT-βgeo and an empty vector control (HSVPrPUC). This specific amplicon-based platform as well as platforms having the features described here (e.g., a promoter driving a fusion transgene flanked by SB inverted/direct repeats) can be used to treat prenatal and/or perinatal brain diseases and other birth defects.

The compositions described herein can be used to reduce the severity of a birth defect that manifests as a structural, functional, or metabolic disorder or abnormality.

For example, the birth defect treated may be due to an inborn error of metabolism. For example, the defect can result from a deficiency of an essential protein, such as an enzyme or hormone. Tay-Sachs disease results when affected babies lack an enzyme (hexosaminidase A (Hex-A)) that catalyzes the breakdown of certain fatty substances in neurons. Thus, where treatment for Tay-Sachs disease is contemplated, the vehicles of the invention can include a sequence that encodes Hex-A or a biologically active variant thereof (e.g., a fragment or other mutant of Hex-A), and the methods of the invention can include administering a therapeutically effective amount of that vehicle to a mammal (e.g., a human) in utero or as soon as a diagnosis has been made in the peri- or postnatal period. The biologically active variant can encode a fragment or other mutant of Hex-A that alleviates a sign or symptom of Tay-Sachs disease (e.g., expression of the variant may reduce the severity of, delay the onset of, or slow the progression of a sign or symptom of the disease). Variants used to treat other birth defects may be similarly assessed, and may mildly, moderately, or significantly improve the disease or prevent it. Accordingly, the methods of the invention include treating Tay-Sachs disease by using a vehicle of the invention to deliver a functional Hex-A polypeptide. Where we describe other specific deficiencies to illustrate the invention, it is to be understood that vehicles that compensate for the stated deficiency and methods of treating that deficiency are within the scope of the present invention. For example, the invention includes a vehicle as described herein configured to express (e.g., comprising a sequence that encodes) galactocerebrosidase and methods of using those vehicles to treat Krabbe's disease, as described further below.

Another enzyme-related birth defect is referred to as Krabbe's disease (also known as Krabbe's leukodystrophy or galactosylceramide β-galactosidase deficiency), which is a rare inherited lipid storage disease in which the enzyme galactocerebrosidase (GALC) is deficient. The result is demyelination. The vehicles of the invention can include a sequence that encodes the deficient polypeptide (e.g., an enzyme) or a biologically active variant thereof (e.g., a fragment or other mutant that, upon expression, results in a clinically significant improvement in demyelination). The “sequence”, regardless of the precise disease indication for which it is mentioned, may be referred to as a “heterologous sequence” or “transgene”. As with Tay-Sachs disease, an appropriate vehicle can be administered in utero or at any time following diagnosis. In any of the methods, the amount of the vehicle can be described as a therapeutically effective amount.

Enzymatic activity is also affected in Gaucher's disease, where a glucocerebrosidase deficiency can result in accumulation of glucocerebroside in the spleen, liver, lungs, bone marrow, and brain. Thus, the invention includes compositions and methods for treating or preventing (e.g., reducing the severity of) Gaucher's disease. This defect is categorized according to severity. Type 1 is the most common form, and patients in this group usually bruise easily and experience fatigue due to anemia and low blood platelets. They also have an enlarged liver and spleen, skeletal disorders, and, in some instances, lung and kidney impairment. There are no signs of brain involvement. As symptoms can appear at any age, treatment can begin at any age. In type 2 Gaucher disease, liver and spleen enlargement are apparent by three months of age. Patients have extensive and progressive brain damage and usually die by two years of age. In the third category, type 3, liver and spleen enlargement is variable, and signs of brain involvement such as seizures gradually become apparent. The compositions and methods of the invention can be used to treat Gaucher patients by delivering a therapeutically effective amount of a transgene that encodes glucocerebrosidase or a biologically active fragment or other mutant thereof that facilitates breakdown and recycling of glucocerebroside.

Phenylketonuria (PKU) is another metabolic disorder. Babies affected by PKU cannot process certain proteins due to a lack of phenylalanine hydroxylase. Thus, where treatment for PKU is contemplated, the vehicles of the invention can include a sequence that encodes phenylalanine hydroxylase or a biologically active variant thereof, and the methods of the invention can include administering that vehicle to a mammal (e.g., a human) in utero or as soon as a diagnosis has been made in the peri- or postnatal period. As noted generally elsewhere, where a specific defect is described to illustrate the invention (here, PKU), it is to be understood that the invention encompasses vehicles, including vectors contained within pharmaceutically acceptable compositions, for the treatment of the defect, methods of treating a patient diagnosed as having that defect, the use of compositions (e.g., herpes viruses comprising modified artificial chromosomes) in the treatment or prevention of a birth defect, and use of the present compositions in the preparation of a medicament for the treatment or prevention of a birth defect.

Other defects are causally associated with a defective membrane channel (e.g., a chloride channel) or receptor. For example, cystic fibrosis (CF) results from the lack of a functional cystic fibrosis transmembrane conductive regulator (CFTR). In that condition, salts and water do not traverse the cell membrane normally and thick secretions form in the respiratory and digestive tracts. The vehicles of the invention can include a sequence that encodes CFTR or a biologically active variant thereof, and the methods of the invention include those for treating CF by administering that vehicle to a mammal (e.g., a human) in utero or as soon as a diagnosis has been made in the peri- or postnatal period. While it is preferable that the compositions and methods of the invention essentially eliminate the defect, compositions and methods that achieve less but still improve the patient's condition are also useful and are within the scope of the present invention. The present compositions can be used in conjunction with currently known therapies for any of the respective birth defects.

Thalassemia can also be treated, and the compositions of the invention include any of the herpes virus-based vectors described herein that include a transgene that encodes a protein deficient in this condition. The two primary types of thalassemia, alpha and beta, result when one or more of the four genes needed for making the alpha globin chain of hemoglobin are variant or missing. Moderate to severe anemia results when more than two genes are affected. Alpha thalassemia major can result in miscarriages. Beta thalassemia occurs when one or both of the two genes needed for making the beta globin chain of hemoglobin are variant. The severity of illness depends on whether one or both genes are affected, and the nature of the abnormality. If both genes are affected, anemia can range from moderate to severe. Accordingly, one can administer, by way of the compositions described herein, a nucleic acid sequence encoding the alpha globin chain, the beta globin chain of hemoglobin, or variants thereof that retain sufficient biological activity to improve the anemia that would otherwise typically result. As with other birth defects, the treatment can begin in utero, within the perinatal period, or as soon as the diagnosis is made. The methods of the invention can include the step of diagnosing the birth defect and the subject to be treated can be monitored periodically for signs of improvement.

The birth defects described here with particularity are representative examples of the defects that can be treated. In any instance where a birth defect is associated with a deficiency in protein expression (e.g., a lack of expression, diminished expression or expression of a dysfunctional protein), a herpes virus amplicon particle can be used to deliver a sequence encoding a functional (or more functional) protein to an affected cell, and such particles are within the scope of the present invention. For example, another sequence that can be incorporated into the compositions of the invention encodes the enzyme GUS-B, and another birth defect that can be treated is Canavan's disease. The herpes virus amplicon particle can be engineered to integrate the sequence carried by the amplicon into the genome of the host cell and the amplicon particle can be made by a helper virus-free method. Such particles, including a transgene that expresses a polypeptide or RNA that compensates for a protein or gene defect that is causally associated with the birth defect, are within the scope of the present invention, as are isolated or purified cells into which the amplicon particle has been introduced. Pharmaceutically acceptable compositions comprising these amplicon particles and kits are also within the scope of the present invention.

As noted above, altered herpes viruses that have packaged modified artificial chromosomes can be used to screen for therapeutic agents that can be developed and used to reduce the severity of a birth defect. These altered herpes viruses can also be used to identify a cellular target for therapeutic intervention during the prenatal or perinatal periods.

More specifically, we have found that targeting vectors containing certain elements of herpes viruses can be used to generate modified artificial chromosomes. These chromosomes, which include a transgene, can then be packaged into herpes virus particles, and the particles can be used for functional genomic studies of birth defects and in therapeutics thereof (including use in the preparation of a medicament). While some birth defects have been causally associated with a deficiency in a single, defined gene, many birth defects appear to be caused by abnormalities in a combination of one or more genes and/or environmental factors (i.e., there is multifactoral inheritance). These birth defects include cleft lip and cleft palate, clubfoot, some heart defects, and spina bifida. Other birth defects appear to have resulted solely from exposure to a teratogen, such as thalidomide or a retinoic acid.

To identify therapeutic agents useful in treating these birth defects, one can provide cells from an animal model of the defect; expose the cells to one or more transgenes carried by an altered herpes virus, and determine whether the transgene(s) ameliorate(s) the birth defect. Some animal models are known in the art. For example, the anticonvulsant sodium valproate (VPA) has been reported to be a teratogen, causing neural tube defects in 1% to 2% of exposed fetuses (Robert and Rosa, Lancet 2:937, 1982). A number of other defects are also induced by valproic acid treatment during pregnancy (Nau et al., J. Pharmacol. Exp. Ther., 219:768-777, 1981; see also Ehlers et al., 1992a 1992b). Accordingly, one can generate a model of a birth defect by exposing a cell to VPA, thalidomide, a retinoic acid, or any other known or suspected teratogen, including those listed in Appendix A, for a time and under conditions sufficient to allow the teratogen to adversely affect the cell. The cell can be a cell from an established cell line, a primary cell placed in culture, or a cell in vivo (i.e., whole animal models (e.g., rodents or non-human primates) can be used in the screening methods of the present invention. The cells of the cell line can be human and may be established from any given tissue (e.g., kidney, muscle, or brain). The primary cells may also be human. Regardless of whether the screen is carried out in tissue culture or in vivo, the cells can be exposed to one or more altered herpes viruses and examined to determine whether the transgene(s) carried by the herpes virus prevents or ameliorates the adverse effect of the teratogen on the cell. If so, the transgene and biologically active variants thereof are potential therapeutic agents useful in treating the birth defect(s) caused by the teratogen in question (i.e., the teratogen to which the cells were exposed).

The targeting vectors and modified artificial chromosomes can be made from existing artificial chromosomes or generated de novo. Methods for incorporating the modified artificial chromosomes into herpes viruses are described further below, and the resulting, altered herpes viruses can be configured in an array to carry out the screening methods of the invention. For example, cells or tissues obtained from an animal (e.g., a non-human animal such as a rodent or non-human primate) having a birth defect can be distributed in the wells of a multi-well tissue culture plate or other compartmentalized device containing altered herpes viruses that include distinct heterologous sequences. Where a single altered herpes virus includes, as its heterologous sequence, more than one gene sequence, the heterologous sequence can be reduced, if desired, until the minimal effective sequence is identified. In other configurations, cells or tissues can be distributed in the wells of a multi-well tissue culture plate or other compartmentalized device and exposed, simultaneously or sequentially, to one or more teratogens and altered herpes viruses that include distinct heterologous sequences. In addition to the screening methods per se, the compositions useful in carrying out these methods (e.g., a herpes virus comprising a modified artificial chromosome and cells (e.g., cells in culture, which may be configured in arrays) are also within the scope the present invention.

While cells within an array can be useful for, for example, high-throughput screening, cells in other configurations (e.g., homogeneous or heterogeneous populations of cells in tissue or organ cultures or in vivo) can also be screened.

Following transduction of a cell with an altered herpes virus, one can also determine whether a given transgene encodes a protein that affects a therapeutic target, thereby identifying the therapeutic target. For example, if cells affected by a genetic abnormality that gives rise to a birth defect are exposed to an altered herpes virus, and a gene sequence carried by that herpes virus encodes a polypeptide that ameliorates the birth defect by, for example, binding to and activating a cell surface receptor, then that receptor is a therapeutic target and other agents (e.g., antibodies or small molecules) that similarly affect the receptor can be used to treat the birth defect. The therapeutic target may be a primary target, which is directly affected by the transgene product (e.g., a receptor is a primary target where the transgene product binds and alters (e.g., stimulates or inhibits) the receptor's activity). The therapeutic target can also be a secondary target, which is one that operates in the same biochemical pathway as the primary target. For example, if a transgene product binds and inhibits a receptor's activity in a therapeutically beneficial way, one could then readily design therapeutic agents that inhibit one or more of the proteins that are active in the signal transduction pathway between the receptor and the effector (i.e., one or more of the secondary targets). Once a target has been identified, one can make and use therapeutic agents other than those encoded by the transgene. For example, where a therapeutically effective transgene encodes a receptor antagonist, one can, if desired, use receptor antagonists other than the one encoded by the transgene. For example, one could use a ligand engineered to bind the receptor but inhibit signal transduction or an antibody that specifically binds and inhibits the receptor. Other agents that inhibit the target by inhibiting its expression can also be administered (e.g., antisense oligonucleotides or siRNAs or other molecules that mediate RNAi). Similarly, where a therapeutically effective transgene encodes an enzyme, such as HEX-A, galactocerebrosidase, or any other enzyme causally associated with a birth defect or another agent that achieves the same result. For example, one can administer an expression construct that is not herpes virus-based (e.g., a plasmid) but that encodes the enzyme or a biologically active variant or fragment thereof.

We use the term “protein(s)” to refer to polymers of naturally or non-naturally occurring amino acid residues, whether glycosylated or not, and whether otherwise post-translationally modified or not. We may also refer to these polymers as “polypeptides” or “oligopeptides” or “peptides”.

While the screening methods of the invention are described further below, we note here that a library of altered herpes viruses that express various nucleic acid sequences (e.g., genomic or cDNA sequences from a human or another organism, such as a plant) can be used to identify genes important for a variety of physiological events (e.g., cell division, signal transduction, hormone production and secretion, motility, differentiation, muscle contraction, energy production, metabolism, neuroprotection or neuroregeneration). Using the methods described here, one can retrofit any library of existing artificial chromosomes so they can be converted into, or packaged within, herpes virus virions. Alternatively, one can generate new libraries of artificial chromosomes that can be packaged by virions. The retrofit includes inserting, preferably into each clone within the library (e.g., a BAC library), a cleavage/packaging signal (also known as an a sequence/segment or pac) and an ori (the origin of replication, also referred to as a c region) from a herpes virus. Generating new libraries requires providing parental vectors that include the a sequence and an ori, and using those vectors to generate a library of artificial chromosomes. Once the transgenes from the artificial chromosomes are packaged in the virions, cells can be transduced with the virions and examined to determine whether the transgene affects or alters a cellular process (e.g., cell survival, the rate of cell division, cell fate, regenerative ability, or any of the other cellular processes referred to herein and affected in the context of a birth defect).

In view of the present description, it will be understood that the invention features methods of reducing the severity of a birth defect in a mammal by, inter alia, exposing the mammal (e.g., in utero) to a herpes virus amplicon particle comprising a cis element-flanked transgene and a sequence encoding a transposase, wherein, upon expression, the transposase inserts the transgene into the genome of a cell (e.g., a neuron) within the mammal and the transgene expresses a polypeptide or RNA that compensates for a protein or gene defect that is causally associated with the birth defect. The mammal can be a human, and the protein that is causally associated with the birth defect can be an enzyme (e.g., hexosaminidase A or phenylalanine hydroxylase) or hormone. In any of the methods, the sequence encoding the transposase can be Sleeping Beauty or a biologically active variant or mutant thereof, and the herpes virus amplicon particle can be made by a helper virus-free method. Where the transgene expresses an RNA, it can be an RNA that mediates RNAi and compensates for a protein by mitigating the expression or activity of the protein.

Other methods can be carried out to determine whether a polypeptide or RNA compensates for a protein or gene defect that is causally associated with a birth defect in a mammal (e.g., a human). These methods can include the steps of: (a) providing a cell of a mammal, wherein the cell exhibits an abnormality exhibited by cells affected by the birth defect; (b) exposing the cell to a herpes virus comprising a modified artificial chromosome, wherein the cell is exposed to the herpes virus for a time and under conditions in which the herpes virus transduces the cell and a nucleic acid sequence carried by the artificial chromosome is expressed as an RNA or polypeptide within the cell; and (c) determining whether the RNA or polypeptide favorably alters the abnormality and thereby compensates for a protein that is causally associated with a birth defect. In this context as well, the protein that is causally associated with the birth defect can be an enzyme (e.g., hexosaminidase A or phenyalanine hydroxylase) or hormone. The cell can be one positioned in vivo or a cell in cell culture, and can be of any type (e.g., a neuron) or at any stage of differentiation (e.g., a neural precursor). The modified artificial chromosome can include: (a) a pair of cleavage sites that flank (i) a packaging/cleavage site of a herpes virus; (ii) an ori of a herpes virus; (iii) a first antibiotic resistance gene; and, optionally (iv) a sequence that encodes a detectable marker; (b) the nucleic acid sequence; and, optionally (c) a second antibiotic resistance gene. The herpes virus can be a herpes simplex virus, varicella zoster virus, Epstein-Barr virus, or cytomegalovirus, and the herpes simplex virus can be of any type (e.g., type 1 (HSV-1), type 2 (HSV-2), type 3 (HSV-3), type 4 (HSV-4), type 5 (HSV-5), type 6 (HSV-6), type 7 (HSV-7), or type 8 (HSV-8) herpes simplex virus). Where RNA is expressed, the RNA can mediate RNAi and compensate for a protein by mitigating the expression or activity of the protein.

Regarding use, the invention features the use of a herpes virus comprising a modified artificial chromosome, as described herein, in the treatment of (e.g., to reduce the severity of) a birth defect. The artificial chromosome includes a nucleic acid sequence that, when expressed as an RNA or polypeptide within a cell, compensates for a protein that is causally associated with the birth defect. Also featured is the use of a herpes virus comprising a modified artificial chromosome in the preparation of a medicament for the treatment of a birth defect, as described herein. The artificial chromosome includes a nucleic acid sequence that, when expressed as an RNA or polypeptide within a cell, compensates for a protein that is causally associated with the birth defect.

Other features and advantages of the invention will be apparent from the drawings, the following detailed description, and from the claims.

### BRIEF DESCRIPTION OF THE DRAWINGS

FIG. 1 is a schematic representation of a method that can be used to generate a modified artificial chromosome.

FIG. 2 is a schematic representation of an FRT site. The sequence of the FRT site is composed of three 13-bp symmetry elements (horizontal elements labeled a, b, and c) surrounding an asymmetrical 8-bp core (open box). FLP-mediated cleavage sites are indicated by two small vertical arrows.

FIG. 3 is a Table of essential HSV-1 genes.

FIGS. 4A and 4B are schematic representations of the HSV-1 genome and the overlapping set of five cosmids C6Δa48Δa (cos 6Δa, cos 28, cos 14, cos 56, and cos 48Δa; Fraefel et al., J. Virol. 70:7190-7197, 1996). In the HSV-1 genome of FIG. 5A, only the IE4 gene, oriS and oriL are shown. The a sequences, which contain the cleavage/packaging sites, are located at the junction between the long and short segments and at both termini. In FIG. 5B, the deleted a sequences in cos 6Δa and cos 48Δa are indicated by “X”.

FIG. 5 is a schematic representation of a bipartite integrating HSV amplicon vector system. HSVPrPUC, which harbors the HSV immediate-early 4/5 gene (IE4/5) promoter and a multiple cloning site, served as an empty vector control and has been described by Geller et al. (Proc. Natl. Acad. Sci. USA 87:8950-8954, 1990). HSVsb was constructed using HSVPrPUC as the plasmid backbone to express high transient levels of the SB transposase under the transcriptional control of the HSV IE4/5 promoter. The second amplicon served as the substrate vector for the transposase and carried a terminal inverted/direct repeat-flanked transgene segment (termed “transgenon”), which expressed a f-galactosidase-neomycin resistance gene fusion under Rous sarcoma virus (RSV) long terminal repeat transcriptional control (HSVT-βgeo). Following construction, all three amplicon vectors were packaged into HSV virions using a previously described helper virus-free methodology (Bowers et al., Gene Ther. 8:111-120, 2001) for in vitro and in vivo assessment.

FIG. 6 is a graph depicting integration of HSV amplicon vectors in BHK cells. Monolayers of BHK cells were left untreated or were transduced with 5×10⁴ virions of HSVsb alone, HSVT-βgeo alone, or HSVT-βgeo plus HSVsb. Three days later, cultures were placed under G418 selection, which was continued for 2 weeks to allow for colony growth. Resultant G418-resistant colonies were stained with X-gal and enumerated. * means that the difference between HSVT-βgeo alone and HSVT-βgeo plus HSVsb treatment was statistically significant (p<0.05).

FIGS. 7A and 7B are graphs depicting cotransduction of primary neuronal cultures with HSVT-βgeo and HSVsb. The cotransduction resulted in enhanced gene expression and retention of transgenon DNA. In FIG. 7A, primary neuronal cultures established from E15 C57BL/6 mouse embryos were transduced at 4 days in vitro with HSVsb and/or HSVT-βgeo and analyzed at day 4 or 9 posttransduction by enumeration of lacZ-positive cells following X-gal histochemistry. In FIG. 7B, quantitation of retained transgenon DNA sequences was quantitated using real-time quantitative PCR. * means that the difference between HSVT-βgeo alone and the HSVT-βgeo plus HSVsb combination group was statistically significant (p<0.05).

FIG. 8 is a schematic representation of integration sites of the viral constructs. Cotransduction of primary neuronal cultures with HSVT-βgeo and HSVsb results in integration of transgenon sequences into transduced cell DNA. Inverse PCR was performed to determine novel flanking sequences of the integrated transgenon in primary neuronal cultures using three nested sets of PCR primers. Amplified DNA segments were isolated, cloned into plasmids and sequenced. Vector/genome junction regions, including the mouse-derived flanking sequences and corresponding GenBank accession numbers are depicted for both the 5′ and the 3′ junctions.

FIG. 9 is a series of photomicrographs demonstrating that in utero co-delivery of HSVT-βgeo and HSVsb to E14.5 mouse CNS results in transgenon expression throughout the brain 97 days post-transduction. A 2-μl bolus (2×10⁴ total transducing units) of a 1:1 mixture of HSVsb+HSVT-βgeo or HSVPrPUC+HSVT-βgeo was administered to the brains of E14.5 C57BL/6 mouse embryos and the animals were allowed to develop to term. At 90 days of age, inoculated animals were sacrificed, perfused with 4% paraformaldehyde, brain sections processed for LacZ/Diaminobenzidine (DAB) immunohistochemistry, and sections imaged using light microscopy (n=8 per treatment group). To illustrate the widespread expression patterns arising from the T-βgeo transgenon, eight representative coronal brain sections from each of three mice receiving HSVsb+HSVT-βgeo are depicted in series from rostral to caudal regions. Stained regions, which indicate areas of βgeo expression, were equivalently maximized across sections for visualization purposes by PHOTOSHOP™-mediated enhancement of the blue color channel. Magnification=1.25×.

FIG. 10 is a series of photomicrographs demonstrating in utero co-delivery of HSVT-βgeo and HSVsb to E14.5 mouse CNS results in prolonged transgenon expression primarily in NeuN-positive neurons of the brain. A 2-μl bolus (2×10⁴ total transducing units) of a 1:1 mixture of HSVsb+HSVT-βgeo or HSVPrPUC+HSVT-βgeo was administered to the brains of E14.5 C57BL/6 mouse embryos and the animals were allowed to develop to term. At 90 days of age, inoculated animals were sacrificed, perfused with 4% paraformaldehyde, brain sections processed for dual LacZ/NeuN or LacZ/GFAP immunocytochemistry, and sections imaged using confocal microscopy (n=8 per treatment group). Representative brain sections corresponding to the cortex, dentate gyrus, and the CA1 region of the hippocampus are depicted. LacZ-specific staining results from T-βgeo transgenon-mediated expression appears in the green channel, GFAP-positive astrocytes and NeuN-positive neurons appear in the red channel, while co-localized staining (Merge) appears as yellow. Magnification=40×.

FIG. 11 is a series of photomicrographs demonstrating β-galactosidase-expressing neuronal precursor cells observed in the neurogenic regions of the brains from adult mice intraventricularly transduced with HSVsb+HSVT-βgeo at E14.5. At 90 days of age, inoculated animals were sacrificed and perfused with 4% paraformaldehyde; brain sections processed for dual lacZ with precursor marker DCX (a-d), TuJ1 (e-h), S100B (i-1), or NG2 (m-p) immunocytochemistry; and sections imaged using confocal microscopy (n=8 per treatment group). Representative brain sections corresponding to the ventricular zones are depicted. The “Merged” panels represent colocalized staining of LacZ-specific staining resulting from βgeo transgenon-mediated expression and precursor cell markers. Original magnification was 40× for all images except d, h, 1 and p, for which photomicrographs were taken at 100× magnification to reveal more morphological detail.

### DETAILED DESCRIPTION

The compositions described herein can be used, as appropriate, to reduce the severity of a birth defect in a mammal. The treatment methods can include the steps of: exposing the mammal, in utero, to a herpes virus amplicon particle comprising a cis element-flanked transgene and, optionally, a sequence encoding a transposase. Upon expression, the transposase inserts the transgene into the genome of a cell within the mammal and the transgene expresses a polypeptide or RNA that compensates for a protein or gene defect that is causally associated with the birth defect. The RNA can be selected to mediate RNAi and would compensate for a protein by mitigating the expression or activity of the protein. The use of an inhibitory substance, such as an siRNA or an shRNA, would be appropriate where birth defects result from overexpression of one or more gene products as occurs, for example, in trisomy 13, trisomy 18, and trisomy 21 (which manifests as Down Syndrome).

Methods for generating herpes virus amplicon particles are known in the art, and the particles used to express an RNA or polypeptide that ameliorates a sign or symptom of a birth defect can be produced by helper virus-free methods.

Methods for generating helper virus-free Herpesvirus amplicons: Generally, the therapeutic compositions of the invention can be made by transfecting a host cell with several vectors and then isolating HSV amplicon particles produced by the host cell (while the language used herein may commonly refer to a cell, it will be understood by those of ordinary skill in the art that the methods can be practiced using populations (whether substantially pure or not) of cells or cell types, examples of which are provided elsewhere in our description). The method for producing an hf-HSV amplicon particle can be carried out, for example, by co-transfecting a host cell with: (i) an amplicon vector comprising an HSV origin of replication, an HSV cleavage/packaging signal, and a heterologous transgene expressible in a cell; (ii) one or more vectors that, individually or collectively, encode all essential HSV genes but exclude all cleavage/packaging signals; and (iii) a vhs expression vector encoding a virion host shutoff protein. One can then isolate or purify (although absolute purity is not required) the HSV amplicon particles produced by the host cell. When the HSV amplicon particles are harvested from the host cell medium, the amplicon particles are substantially pure (i.e., free of any other virion particles) and present at a concentration of greater than about 1×10⁶ particles per milliliter. To further enhance the use of the amplicon particles, the resulting stock can also be concentrated, which affords a stock of isolated HSV amplicon particles at a concentration of at least about 1×10⁷ particles per milliliter.

The amplicon vector can either be in the form of a set of vectors or a single bacterial-artificial chromosome (“BAC”), which is formed, for example, by combining the set of vectors to create a single, doublestranded vector. As noted above, methods for preparing and using a five cosmid set are disclosed in, for example, Fraefel et al. (J. Virol, 70:7190-7197, 1996), and methods for ligating the cosmids together to form a single BAC are disclosed in Stavropoulos and Strathdee (J. Virol. 72:7137-43, 1998). The BAC described in Stavropoulos and Strathdee includes a pac cassette inserted at a BamHI site located within the UL41coding sequence, thereby disrupting expression of the HSV-1 virion host shutoff protein.

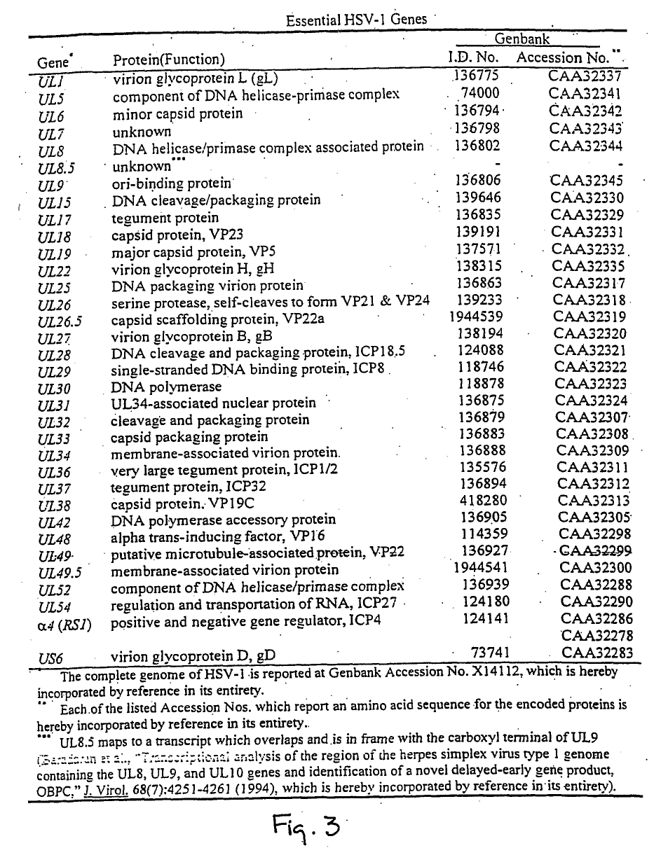

By “essential HSV genes”, it is intended that the one or more vectors include all genes that encode polypeptides that are necessary for replication of the amplicon vector and structural assembly of the amplicon particles. Thus, in the absence of such genes, the amplicon vector is not properly replicated and packaged within a capsid to form an amplicon particle capable of adsorption. Such “essential HSV genes” have previously been reported in review articles by Roizman (Proc. Natl. Acad. Sci. USA 93: 11313-8, 1996; Acta Viroloeica 43:75-80, 1999). Another source for identifying such essential genes is available at the Internet site operated by the Los Alamos National Laboratory, Bioscience Division, which reports the entire HSV-1 genome and includes a table identifying the essential HSV-1 genes. The genes currently identified as essential are listed in the Table provided as FIG. 3.

In other embodiments, a helper-free herpesvirus amplicon particle (e.g., an hf-HSV) can be generated by: (1) providing a cell that has been stably transfected with a nucleic acid sequence that encodes an accessory protein (alternatively, a transiently transfected cell can be provided); and (2) transfecting the cell with (a) one or more packaging vectors that, individually or collectively, encode one or more (and up to all) HSV structural proteins but do not encode a functional herpesvirus cleavage/packaging site and (b) an amplicon plasmid comprising a sequence that encodes a functional herpesvirus cleavage/packaging site and a herpesvirus origin of DNA replication (ori). The amplicon plasmid described in (b) can also include a sequence that encodes a therapeutic agent. In another embodiment, the method comprises transfecting a cell with (a) one or more packaging vectors that, individually or collectively, encode one or more HSV structural proteins (e.g., all HSV structural proteins) but do not encode a functional herpesvirus cleavage/packaging site; (b) an amplicon plasmid comprising a sequence that encodes a functional herpesvirus cleavage/packaging site, a herpesvirus origin of DNA replication, and a sequence that encodes an immunomodulatory protein (e.g., an immunostimulatory protein), a tumor-specific antigen, an antigen of an infectious agent, or a therapeutic agent (e.g., a growth factor); and (c) a nucleic acid sequence that encodes an accessory protein.

The HSV cleavage/packaging signal can be any cleavage/packaging that packages the vector into a particle that is capable of adsorbing to a cell (the cell being the target for transformation). A suitable packaging signal is the HSV-I “a” segment located at approximately nucleotides 127-1132 of the a sequence of the HSV-I virus or its equivalent (Davison et al., J. Gen. Virol. 55:315-331, 1981).

The HSV origin of replication can be any origin of replication that allows for replication of the amplicon vector in the host cell that is to be used for replication and packaging of the vector into HSV amplicon particles. A suitable origin of replication is the HSV-I “c” region, which contains the HSV-I ori segment located at approximately nucleotides 47-1066 of the HSV-I virus or its equivalent (McGeogh et al., Nucl. Acids Res. 14:1727-1745, 1986). Origin of replication signals from other related viruses (e.g., HSV-2 and other herpes viruses, including those listed above) can also be used.

The amplicon plasmids can be prepared (in accordance with the requirements set out herein) by methods known in the art of molecular biology. Empty amplicon vectors can be modified by introducing, at an appropriate restriction site within the vector, a complete transgene (including coding and regulatory sequences). Alternatively, one can assemble only a coding sequence and ligate that sequence into an empty amplicon vector or one that already contains appropriate regulatory sequences (promoter, enhancer, polyadenylation signal, transcription terminator, etc.) positioned on either side of the coding sequence. Alternatively, when using the pHSVlac vector, the LacZ sequence can be excised using appropriate restriction enzymes and replaced with a coding sequence for the transgene. Conditions appropriate for restriction enzyme digests and DNA ligase reactions are well known in the art (see, e.g., Sambrook et al., Molecular Cloning: A Laboratory Manual, Cold Spring Laboratory, Cold Spring Harbor, N.Y. (1989); Ausubel et al. (Eds.), Current Protocols in-Molecular Biology, John Wiley & Sons, New York, N.Y., 1999 and preceding editions; and U.S. Pat. No. 4,237,224).

We now further describe the targeting vectors and other compositions of matter that can be variously used to practice the methods of the invention. Manipulation of the targeting vectors by the methods described below gives rise to altered herpes viruses that can be used to screen for therapeutic agents useful in the treatment of birth defects and to identify therapeutic targets in that context.

Targeting vectors and precursors thereof The vectors we describe as targeting vectors can be made from nucleic acids and, in form, may be linear or circular. For example, the targeting vectors can be plasmids (single- or double-stranded, circularized DNA or RNA molecules). A circularized vector such as a plasmid can be converted to a linear vector by cleaving it at one or more locations. For example, a plasmid can be cleaved at one or more restriction sites or cleavage sites. Alternatively, a linear targeting vector can be made by methods known in the art. For example, one can synthesize and anneal sense and antisense strands of DNA or RNA.

With respect to content, the nucleic acid sequences within the targeting vectors can include a packaging/cleavage site of a herpes virus and an ori of a herpes virus. The packaging/cleavage signal can be any sequence that directs the vector into a particle that is capable of adsorbing to a cell (the cell being the target for transformation). Where the targeting vectors are linear and intended for insertion into a unique or particular site within an artificial chromosome, it is unlikely that any other elements need be present in the targeting vector. Where the targeting vectors participate in reactions where unwanted constructs may form, however, it is beneficial to include additional elements within the targeting vectors that facilitate selection or detection of the modified artificial chromosomes. For example, the ability to discern among possible recombinants can be facilitated by the use of a selectable marker carried with the targeting vector. Accordingly, in addition to the packaging/cleavage site and the ori, a targeting vector can include a sequence that encodes a selectable marker (e.g., an antibiotic resistance gene) and/or a sequence that encodes a detectable marker (e.g., a fluorescent protein). Additional elements may also be present, as may sequences that constitute the backbone of the vector.

The packaging/cleavage site can be that of any herpes virus or a biologically active fragment or other mutant thereof that retains sufficient biological activity to remain useful in the methods of the invention. The a sequence varies in size from 280 to 550 bp among HSV-1 strains and contains unique and directly repeated sequence elements. Similarly, the ori can be that of any herpes virus or an active fragment or other mutant thereof (e.g., a variant that retains the ability to mediate replication of nucleic acid sequences).

In specific embodiments, the packaging/cleavage site can be that of HSV-1. Other sequences can be found in the literature or in publicly available databases such as GenBank™. The ori can also be that of an HSV-1. More generally, in any of the compositions described herein that include a packaging/cleavage site and an ori, these elements can be, independently, those of any of the more than 100 known species of herpes virus. For example, the cleavage/packaging site and the ori can be those of an alpha herpes virus (e.g., a Varicella-Zoster virus, a pseudorabies virus, or a herpes simplex virus (e.g., type 1 or type 2 HSV) or an Epstein-Barr virus). The herpes virus can also be a cytomegalovirus.

Where an HSV element is employed, it can be that of a type 1 (HSV 1) or type 2 (HSV 2) HSV. It can also be that of a type 3 (HSV 3), type 4 (HSV 4), type 5 (HSV 5), type 6 (HSV 6), type 7 (HSV 7), or type 8 (HSV 8) herpes simplex virus. The cleavage/packaging site and the ori can also be those of a human herpes virus. In specific embodiments, the cleavage/packaging site and the ori can be those of HSV 1, and a modified artificial chromosome that incorporates them can be packaged in an HSV 1 virion. In other embodiments, the cleavage/packaging site, the or, and the virus can be HSV 2; and so on.

The selectable marker can be any protein that facilitates separation of the cells that express the marker from the cells that do not. For example, the targeting vector can include a sequence that confers resistance to an antibiotic; cells that express the marker will survive in the presence of the antibiotic, whereas cells that do not express the marker will perish. More specifically, the targeting vectors of the invention can include a sequence encoding a protein that confers resistance to aminopterin, ampicillin, chloramphenicol, erythromycin, kanamycin, hygromycin, spectinomycin, tetracycline, or another antibiotic. The marker may also be a protein that, when expressed, allows a cell to survive in an altered environment. For example, the protein may be a stress protein (e.g., a heat shock protein) that allows a cell to survive in, for example, an environment where the temperature is raised above a normal physiological temperature (e.g., about 37° C.). The targeting vector can include sequences that encode more than one (e.g., two or three) selectable marker, and the advantage of including more than one marker is described further below.

The detectable marker can be essentially any protein; all that is required is that the protein be useful in identifying a cell in which it is expressed. For example, the targeting vectors can include a sequence encoding a protein that is specifically bound by an antibody or other reagent (e.g., a labeled binding partner). The markers may also be detectable by virtue of chemiluminesence or fluorescence. For example, the detectable marker can be a fluorescent protein (e.g., a protein that, upon proper illumination, fluoresces green (e.g., GFP or enhanced GFP (EGFP)), red (e.g., DSred II), or blue). The sequence encoding the detectable marker can be operably linked to a promoter that directs its expression. For example, the promoter can be constitutively active in mammalian cells or cell type-specific. Many such promoters are known and used by those of ordinary skill in the art. As is true for other elements within the targeting vector, the sequence encoding the detectable marker can be incorporated into the modified artificial chromosomes and the virions that package them. For example, the sequence(s) encoding the detectable marker(s) can be flanked by the cleavage sites and recombined with the cleavage/packaging site, the ori, and the sequence encoding the selectable marker into an artificial chromosome.

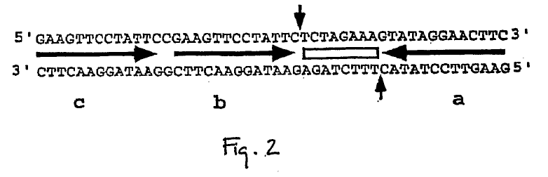

The elements described above (e.g., the herpes virus cleavage/packaging site, the ori, and the sequences encoding the selectable and/or detectable markers) can be flanked by a pair of cleavage sites, which may constitute any sequences that allow for recombination. For example, the cleavage sites can be a pair of LoxP elements, which can reform following cleavage with Cre recombinase, or a pair of Flp recombination targets (FRTs), which can reform following cleavage with Flp recombinase. Each member of the pair of LoxP elements can have, or can include, the sequence 5′-ataacttcgtataatgtatgctatacgaagttat-3′ (SEQ ID NO:1). The minimal sequence of the FRT site is believed to include a 34-basepair sequence containing two 13-basepair imperfect inverted repeats separated by an 8-basepair spacer that includes an Xba I restriction site. An additional 13-basepair repeat is found in most FRT sites, but it may not be required for cleavage. The FRT site serves as a binding site for Flp recombinase (see, e.g., Gronostajski and Sadowski, Mol. Cell. Biol. 5:3274-3279, 1985; Gronostajski and Sadowski, J. Biol. Chem. 260:12320-12327, 1985; and Gronostajski and Sadowski, J. Biol. Chem. 260:12328-12335, 1985). See also, FIG. 2.

As is true for all of the sequences useful in the present invention, the sequences of the cleavage sites can differ from naturally occurring sequences or from elements within commercially available vectors so long as they retain sufficient activity to be useful in the methods of the present invention. For example, the LoxP element can be a fragment or other mutant of a naturally occurring sequence so long as its sequence can still be recognized and cleaved by Cre recombinase. We may describe such fragments and other mutants of specified sequences as having biological activity or as being biologically active. The “cleavage site(s)/sequence(s)” are distinct from the “packaging/cleavage site/sequence.”

Biologically active fragments or mutant sequences can be degenerate variants of a naturally occurring or commercially available sequence. Where the nucleic acid sequences within, for example, the targeting vector or a modified artificial chromosome, encode a protein, at least some of the nucleotides in the third position of the codon can vary but yet encode the same amino acid residue. Biologically active fragments or mutant sequences can also be described as substitution, deletion, or addition mutants, where one or more nucleotides (e.g., 1, 2, 3, 4, 5, or more) are substituted, deleted, or added, respectively. Where the nucleic acid sequence encodes a protein, the biologically active nucleic acid sequence can be altered in such a way that the encoded protein contains a different amino acid residue (e.g., a residue that constitutes a conservative substitution), an additional amino acid residue, or fewer amino acid residues.

In specific embodiments, the targeting vectors of the invention include at least one (e.g., one) pair of cleavage sites, one or more cis elements from a herpes virus (e.g., a packaging/cleavage site and/or an ori), a sequence encoding a selectable marker (e.g., an antibiotic resistance gene) and, optionally, a sequence encoding a detectable marker (e.g., a detectable label or tag). A pair of cleavage sites can flank either all or various cis elements and the sequences encoding the selectable and detectable markers. For example, in one embodiment, the targeting vector includes a single pair of cleavage sites that flank a packaging/cleavage site of a herpes virus, an ori of a herpes virus, and an antibiotic resistance gene (e.g., a kanamycin resistance gene (Kan^{r})).

As noted above, the targeting vector can include a second selectable marker that may not lie between a pair of cleavage sites. For example, where the pair of cleavage sites flank a cleavage/packaging site, an ori, and a first resistance gene (e.g., Kan^{r}), the targeting vector may also contain, “outside” the cleavage sites, a second resistance gene (i.e., a gene that confers resistance to an antibiotic other than that to which the first resistance gene is directed). For example, where the first selectable marker is a Kan^{r} sequence, the second selectable marker can be a nucleic acid sequence that confers resistance to aminopterin, ampicillin, chloramphenicol, erythromycin, hygromycin, spectinomycin, or tetracycline.

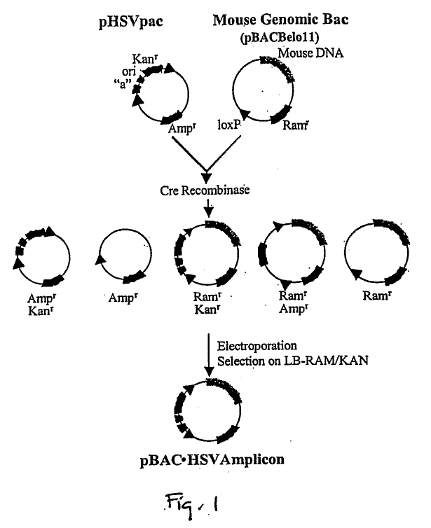

The sequence encoding the second selectable marker may have been present in a vector (e.g., a plasmid (e.g., pBluescript)) used to generate the targeting vector, and certain parental vectors are within the scope of the present invention. For example, the invention features precursor vectors in which either or both of a herpes virus cleavage/packaging site and a herpes virus ori are flanked by unique restriction sites or by a pair of cleavage sites. FIG. 1 is a schematic representation of a method that can be used to generate a modified artificial chromosome. The targeting vector and resulting modified artificial chromosomes, including the pBAC.HSV amplicon and modified artificial chromosomes having the elements of that construct, are within the scope of the present invention.

Using targeting vectors to retrofit an artificial chromosome: Targeting vectors can be used to modify or “retrofit” an artificial chromosome (or a collection thereof) with the a and ori sequences (i.e., to incorporate the a and ori sequences into the artificial chromosome). These two elements are sufficient to confer onto any vector, including the modified artificial chromosomes described herein, the ability to be replicated, cleaved, and inserted into a virion (e.g., an HSV virion). Methods of generating modified artificial chromosomes are described further below. The methods can be carried out by introducing a targeting vector and an artificial chromosome into a cell (e.g., an E. coli strain EL250 containing defective lambda prophage). Those methods, along with methods of inserting the modified chromosomes into virions and using those virions in screening assays and pharmaceutical compositions, are within the scope of the present invention.

We use the term “artificial chromosome” broadly to refer to any non-naturally occurring construct that is capable of incorporating (e.g., into its polymeric structure) large nucleic acid sequences (e.g., sequences greater than about 50 kb). For example, the artificial chromosomes used in the methods of the invention can be yeast artificial chromosomes (YACs), bacterial artificial chromosomes (BACs), and/or human artificial chromosomes (HACs). Within the confines of the upper length limit, these constructs can incorporate essentially any nucleic acid sequence of interest. For example, the constructs can include genomic DNA or cDNA from yeast, bacteria or other pathogens (e.g., viruses, parasites, and fungi), plants (including herbs and particularly including any plant considered to have medicinal properties), or animals. For example, the sequence of interest can be an avian sequence (e.g., a sequence that naturally occurs in a chicken, goose, duck, pheasant, or other bird or a sequence derived therefrom (e.g., a fragment or mutant of an avian sequence)), a reptilian sequence (e.g., a sequence that naturally occurs in a lizard or snake or a sequence derived therefrom (e.g., a fragment or mutant of a reptilian sequence)), an amphibian sequence (e.g., a sequence that naturally occurs in a frog or newt or a sequence derived therefrom (e.g., a fragment or mutant of an amphibian sequence)), or a mammalian sequence (e.g., a sequence that naturally occurs in a sheep, goat, cow, horse, dog, cat, rabbit, pig, human, or rodent (e.g., a rat, mouse, hamster, or guinea pig) or a sequence derived therefrom (e.g., a fragment or other mutant of a mammalian sequence)). Other useful sequences are those of insects (e.g., arthropods), including flies used in research (e.g., D. melanogaster) and other invertebrates (e.g., C. elegans). We may also refer to a nucleic acid sequence of interest as a “transgene.” While artificial chromosomes have the capacity to carry large transgenes, the methods of the invention can be practiced using transgenes of any length.

The artificial chromosomes and modified artificial chromosomes can include more than one transgene that, when expressed, would produce more than one protein or type of protein. For example, the nucleic acid of interest can include several (e.g., 1-5) transgenes that encode several (e.g., 1-5) proteins. For example, the nucleic acid can include transgenes that encode one or more enzymes, receptors, transcription factors, cofactors, extracellular matrix proteins, structural proteins, or other cellular proteins, and the proteins or types of proteins can be the same or different. For example, the nucleic acid of interest can include two transgenes that encode two enzymes, or an enzyme and a structural protein. A given transgene can also be one that encodes an antibody chain or any one of the proteins described herein (see, e.g., the various types and species described above). In the event the nucleic acid of interest within the artificial chromosome or modified artificial chromosome includes more than one transgene, and that nucleic acid produces a desirable effect on a cell, tissue, organ, or animal into which it is introduced (e.g., by way of the modified herpes viruses described herein), one can then isolate and test individual transgenes. For example, one can reduce the size of the nucleic acid (by, for example, exposing it to an endonuclease) so that it encodes only one functional protein or a biologically active fragment thereof. Where one wishes to express a given transgene in a cell, the modified artificial chromosome can be modified to include multiple copies of a transgene.

While it should be clear from the context, we have endeavored to use the term “artificial chromosome” to refer to an artificial chromosome that has not been exposed to, or recombined with elements from, a targeting vector, and the term “modified artificial chromosome” to refer to an artificial chromosome that has been altered to contain desired elements of a targeting vector.

The artificial chromosomes can also include a sequence encoding a selectable marker, which may differ from the selectable marker encoded by the targeting vector. The selectable marker in the artificial chromosome can confer resistance to an antibiotic, including aminopterin, ampicillin, chloramphenicol, erythromycin, kanamycin, hygromycin, spectinomycin, tetracycline, or another antibiotic. For example, the targeting vector can include a sequence encoding a protein that confers resistance to kanamycin, and the artificial chromosome can include a sequence encoding a protein that confers resistance to an antibiotic other than kanamycin (e.g., ampicillin, erythromycin, or tetracycline). When the modified artificial chromosome is generated, it can, then, include two selectable markers. For example, a sequence that confers resistance to neomycin, which can be useful in selecting successfully transduced mammalian cells, and a sequence that confers resistance to ampicillin, which can be useful in selecting successfully transduced bacterial cells (e.g., E. coli).

To generate a modified artificial chromosome, the targeting vector is combined with an artificial chromosome. The artificial chromosomes can contain, as noted above, a sequence of interest and, optionally, a sequence encoding a selectable marker that is distinct from any or all of the selectable markers encoded by the targeting vector. To facilitate recombination, the artificial chromosome can also include at least one cleavage site that is the same as at least one of the cleavage sites in the targeting vector. For example, where the targeting vector includes a pair of LoxP elements, the artificial chromosome can also include a LoxP element. Such targeting vectors and artificial chromosomes can be combined in the presence of Cre recombinase under conditions, and for a time, sufficient to allow the Cre recombinase to cleave the LoxP elements in the targeting vector and the artificial chromosome. Upon recombination, at least some of the reaction products will be configured so that the elements previously flanked by the LoxP sites in the targeting vector will be linked to the sequence of interest (or transgene) and, if present, the sequence encoding the selectable marker gene originally present in the artificial chromosome. These reaction products are within the scope of the present invention, as are pure or substantially pure populations of the desired reaction products.

The desired reaction products can be identified and isolated from other reaction products by transfecting cells (e.g., bacterial cells) with the pool of available reaction products, including the desired construct and those that have recombined in ways that are not useful. The cells can then be grown in the presence of antibiotics, which are chosen in view of the selectable marker genes incorporated in the targeting vector and artificial chromosomes. Where a reaction product includes sequences that confer resistance to the antibiotics, the bacterial cell will survive exposure to the antibiotics. For example, where the cleavage elements of the targeting vector flank Kan^{r} and the artificial chromosome includes Ram^{r}, cells that include modified artificial chromosomes that have recombined in a useful way, and therefore contain both of those resistance genes, will grow on (or in) culture medium containing kanamycin and chloramphenicol.

Targeting vectors that include other cleavage sites can be used to generate modified artificial chromosomes in an analogous way. For example, where the targeting vector includes a pair of FRTs, the artificial chromosome can also include one or more FRTs. Such targeting vectors and artificial chromosomes can be combined in the presence of Flp recombinase under conditions, and for a time, sufficient to allow the Flp recombinase to cleave the FRTs in the targeting vector and the artificial chromosome. Subsequently, the sequence between the FRTs in the targeting vector can be recombined with the FRT-cleaved artificial chromosome.

Targeting vectors and artificial chromosomes that include unique sequences recognized by a restriction endonuclease can also be recombined. For example, the a site and ori can be flanked by sequences that are recognized and cleaved by a restriction endonuclease that does not recognize or cleave the targeting vector at any other site. The modified chromosome can include the same sequence. The digested targeting vectors and artificial chromosomes can then be incubated together in the presence of a ligase. As with any genetic engineering, where the restriction endonuclease generates overhanging (as opposed to blunt) ends, the recombination is likely to be more efficient.

Linear targeting vectors: Instead of a circularized targeting vector, such as a plasmid, one can use a linear targeting vector, which we may also refer to herein as a “cassette”. Thus, the invention encompasses linear, double-stranded targeting vectors that include a cleavage/packaging site, an ori and, optionally, sequences encoding a selectable marker and/or a sequence encoding a detectable marker. The linear cassette can be recombined with an artificial chromosome (or a portion thereof) to generate a modified artificial chromosome. The ends of the linear cassette can be blunt or, to better facilitate recombination, the ends of the sense and antisense strands within the cassette can be staggered and complementary to cleavage sites generated within the artificial chromosome.

The methods employing linear cassettes are similar to those that employ circular targeting vectors; the linear cassette and a linearized artificial chromosome (or a portion thereof (e.g., a portion including a sequence of interest and a selectable marker gene)) are combined under conditions, and for a time, sufficient to allow recombination and the formation of a modified artificial chromosome. Selection can be carried out by transfecting cells (e.g., E. coli) with the resultant constructs, some of which will be properly recombined artificial chromosomes, and culturing the cells in the presence of antibiotics. For example, where the linearized targeting vector includes a sequence that confers resistance to ampicillin and the artificial chromosome (or the portion thereof) includes a sequence that confers resistance to tetracycline, properly modified artificial chromosomes can be selected on the basis of their ability to confer, to cells that contain them, resistance to ampicillin and tetracycline.

The modified artificial chromosomes generated using linear targeting vectors can be packaged in the herpes viruses described herein and used in the screening assays and therapeutic regimes described below (just as if they had been generated using a non-linear targeting vector).

Compositions containing targeting vectors: The targeting vectors can be lyophilized, mixed with a cryoprotectant, or solubilized or suspended in another diluent (e.g., a buffer or alcohol). The compositions can also include preservatives. Such compositions are within the scope of the present invention and may further include an artificial chromosome (as described further below, including those that contain sequences (e.g., cDNA or genomic sequences) of interest from mammals (e.g., humans, mice or other laboratory animals), other animals (e.g., livestock), plants, or pathogens).

Modified artificial chromosomes: The invention features modified artificial chromosomes, including those produced by the methods described here. The modified artificial chromosomes can include (a) a pair of cleavage sites that flank a packaging/cleavage site of a herpes virus; an ori of a herpes virus; and, optionally, a sequence encoding a first selectable marker and/or a sequence that encodes a detectable marker; (b) a nucleic acid sequence of interest; and (c) a sequence encoding a second selectable marker. Typically, the sequence encoding the first selectable marker is derived from the targeting vector (and is therefore flanked by the cleavage sites) and the sequence encoding the second selectable marker is derived from an unmodified artificial chromosome.

As the modified artificial chromosomes can be generated from the targeting vectors and artificial chromosomes described above, the various elements present in the modified artificial chromosomes can be any of those described above. For example, the cleavage sites can be LoxP elements, FRTs, or unique restriction sites; the selectable marker, when present, can be an antibiotic resistance gene (e.g., a sequence that, upon expression, confers resistance to aminopterin, ampicillin, chloramphenicol, erythromycin, hygromycin, kanamycin, spectinomycin, or tetracycline); the sequence of interest can be a genomic or cDNA sequence from a mammalian genome (e.g., the human genome) or the genome of a pathogen (inter alia); and so forth.

Where the modified artificial chromosome is made by methods other than the “recombineering” methods described herein, it may contain fewer elements than described and, in particular, may lack the cleavage sites. Thus, modified artificial chromosomes of the invention can include (e.g., in addition to only their backbone) a packaging/cleavage site of a herpes virus; an ori of a herpes virus; a nucleic acid sequence of interest; and, optionally, sequence encoding a selectable and/or detectable marker. Here, too, these elements can be any of those described in the present specification. Regardless of the precise manner in which the modified artificial chromosome is made, it can be packaged in any of the herpes virus (e.g., a herpes simplex virus, varicella zoster virus, Epstein-Barr virus, or cytomegalovirus). Methods of packaging modified artificial clromosomes are described further below.

In specific embodiments, a targeting vector and an artificial chromosome can be recombined within a cell. The vector and artificial chromosome can be introduced into the cell by methods known in the art, such as calcium phosphate precipitation or electroporation.

Altered herpes viruses: The screening methods to detect therapeutic agents and targets useful in the treatment of birth defects can employ altered herpes viruses that have packaged the modified artificial chromosomes. We may refer to these viruses as particles, and they may package the modified artificial chromosomes described herein. A substantially pure population of the particles can be formulated as compositions, and the particles within the population as well as the manner in which they are formulated, may vary depending upon their intended use (e.g., depending upon whether the particles are intended for use in a screening assay or as therapeutic agents). For example, the compositions may further include one or more diluents (e.g., one or more excipients or carriers).

The altered herpes viruses can infect cells, and an isolated or purified host cell that includes an altered herpes virus that includes a transgene capable of ameliorating a birth defect is within the scope of the present invention. We may refer to host cells as “permissive” for herpes virus propagation. The host cell can be a mammalian cell (e.g., a human cell), and the cell can be one that is maintained in tissue culture. For example, the host cells can be within an organ, tissue, or cell culture. Varying numbers of cells within the organ, tissue, or cell culture may carry the altered herpes virus (complete or uniform transduction is not required). The host cells can also be arrayed on a substrate, and arrays in which cells located in at least one of the positions within the array are infected with a different altered herpes virus than are cells located in at least one other position within the array are also within the scope of the present invention. Regardless of the source of the host cell, it can vary in its developmental stage. For example, mammalian host cells can be embryonic or fetal cells or can be obtained from any age animal (e.g., a young, adolescent, adult, or aged animal).

The altered herpes viruses, in the type of transgene described above, and cells containing them can also be formulated within compositions (e.g., physiologically acceptable compositions), and such compositions are within the scope of the invention. In one embodiment, the composition can include a plurality of altered herpes viruses, all of which (or substantially all of which) express the same transgene. Alternatively, the composition can include a plurality of altered herpes viruses, and the nucleic acid sequence of interest within the modified artificial chromosome of at least one member of the plurality can be different from the nucleic acid sequence of interest within the modified artificial chromosome contained by at least one other member of the plurality. In some embodiments, very few members of the plurality will contain the same transgene (i.e., the plurality can be extremely heterogeneous).

Methods of generating an altered herpes virus: The methods of the invention include methods of generating a herpes virus that includes a modified artificial chromosome or that can package and express a transgene carried by the chromosome. We may refer to these viruses as altered herpes viruses or as herpes virus particles. The methods can be carried out by (a) providing a cell, which may or may not include a nucleic acid sequence that encodes an accessory protein; (b) transfecting the cell with (i) one or more packaging vectors that, individually or collectively, encode one or more of the herpes virus structural proteins but do not include a functional herpes virus ori and (ii) a modified artificial chromosome; and (c) culturing the cell for a time and under conditions that permit the cell to produce an altered herpes virus. In lieu of steps (a) and (b), one may simply obtain the required cell (i.e., steps (a) and (b) may be collapsed into a single “providing” step). The herpes virus can be any of those types referenced above, and the cell can be any permissive cell (e.g., a mammalian cell (e.g., a human cell)). Although the particular cell type is not limited, one could use a neuron, a fibroblast, a blood cell, a hepatocyte, a keratinocyte, a melanocyte, a glial cell, an endocrine cell, an epithelial cell, a muscle cell, a bone cell, a prostate cell, a testicular cell, or a germ cell. The cell may also be diseased (e.g., malignant) and, as noted above, obtained at any developmental stage or at any stage of differentiation.

Where a sequence encoding an accessory protein is employed, that sequence can also encode a biologically active fragment or mutant of an accessory protein (e.g., a biologically active fragment or other mutant of vhs or VP16. The vhs protein has an endoribonucleolytic activity that is important in the time-dependent progression of HSV gene expression and virion assembly, and VP16 is a strong transcriptional activator protein. Any of the invention that include expression of a vhs protein can employ, for example, an HSV-1 vhs protein, an HSV-2 vhs protein, an HSV-3 vhs protein, bovine herpes virus 1 vhs protein, bovine herpes virus 1.1 vhs protein, gallid herpes-virus 1 vhs protein, gallid herpes virus 2 virion hsp, suid herpes virus 1 vhs protein, baboon herpes virus 2 vhs protein, pseudorabies vhs protein, cercopithecine herpes virus 7 vhs protein, meleagrid herpes virus 1 vhs protein, equine herpes virus 1 vhs protein, or equine herpes virus vhs protein). Any of these proteins can be operatively coupled to its native transcriptional control element(s) or to an artificial control element (i.e., a control element that does not normally regulate its expression in vivo).

The sequence encoding VP16 or a transcriptional activator that mimics VP16 can be introduced into packaging cells prior to the packaging components. The activation domain can be replaced with another regulatory protein so long as the signal that regulates the CAT/GRATATGARAT sequences is retained. While “pre-loading” the packaging cells with VP16 is not essential, it can be done within the context of the present methods, and it can lead to an additional enhancement of amplicon particle titers. Moreover, the methods can be carried out with cells in which VP16, or a biologically active variant thereof, is stably expressed (methods to achieve stable expression are known in the art). VHS, or a biologically active variant thereof, can also be stably expressed so long as its expression can be suitably controlled. For example, one can control the expression of a sequence encoding VHS (or a biologically active fragment or other mutant thereof) by placing it in the context of a tetracycline, RU46, or ecdysone system. Similarly, the methods in which herpes virus amplicon particles are generated by transfecting a cell with a sequence encoding VHS can be carried out with VHS (e.g., the VHS encoded by gene UL41) or with a mutant VHS, particularly one in which RNAse activity is reduced. Examples of VHS mutations that lead to abolished RNAse activity are the R27, Sc243, and M384 mutations described previously by Jones et al. (J. Virol. 69:4863-4871, 1995).

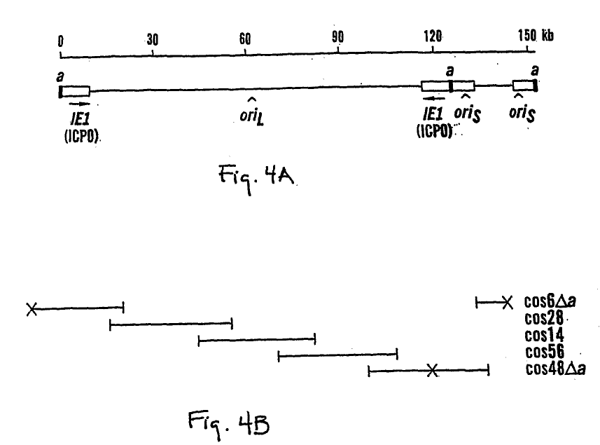

The packaging vectors employed can be a YAC, a BAC, a HAC, an F element plasmid, a cosmid or a set of cosmids. For example, one can use a set of cosmids that, individually or collectively, encode all essential HSV genes but exclude all cleavage/packaging signals. For example, the cosmids can include cos 6Δa, cos 28, cos 14, cos 56, and cos 48Δa (see FIGS. 4A and 4B). Essential HSV-1 genes are listed in the table of FIG. 3.

Methods of producing host cells with stably integrated transgenes: In alternative embodiments, the cell can also be transfected with a sequence encoding an enzyme that catalyzes a reaction within the cell, the consequence of the reaction being that the sequence carried by a herpes virus-based vehicle (e.g., a modified artificial chromosome or herpes virus amplicon particle), such as the transgene, is inserted into the genome of the cell. The enzyme can be, for example, a transposase (e.g., the transposase is encoded by Sleeping Beauty). Although HSV amplicon particles can efficiently infect non-dividing cells and express transgenes therein, long-term expression in actively dividing cells has proven difficult. Combining the Tc1-like Sleeping Beauty (SB) transposon system with the modified artificial chromosomes and packaging vectors described herein can create herpes virus particles that can integrate into the genomes of both dividing and non-dividing cell types. Vector integration within cells can extend the period of expression (e.g., expression of a protein of interest or of a therapeutic agent encoded by a modified artificial chromosome).

To regulate the transposase component of the system more tightly, one could, for example, incorporate the Sleeping Beauty protein into the virion in the form of a fusion with an HSV tegument protein. Alternatively, one could effect exogenous application of transposase protein with the transgenon-containing amplicon vector. Both approaches would prevent continued synthesis of Sleeping Beauty and thus, obviate additional catalysis of transposition. In another approach, the amplicon can be engineered to transiently coexpress host factors known to participate in Sleeping Beauty-mediated transposition to enhance integration into desired regions. One such factor is the highly-conserved DNA-bending protein, HMGB1 (see, e.g., Zayed et al., Nucleic Acids Res. 31:2313-2322). In yet another strategy, one could incorporate protein instability sequences into the open reading frame to limit transposase half-life.

The transposon in the integration vector should be compatible with sequences flanking the transgene in the amplicon plasmid. For example, where the transposon is of the Sleeping Beauty system, the amplicon vector can include a transgene (for integration) flanked by the Sleeping Beauty terminal repeats. Integrating forms of the HSV amplicon vector platform have been described previously. One form consists of an HSV amplicon backbone and adeno-associated virus (AAV) sequences required for integration. Here, an integration system is employed in connection with compositions designed to deliver therapeutic agents such as enzymes, hormones, membrane channels, and inhibitory RNAs (e.g., siRNAs or hairpin RNAs) to cells affected by birth defects.

Isolated altered herpes viruses and compositions containing same: In subsequent steps, the herpes virus particles can be isolated from the cell or from the medium in which the cell was cultured, and such isolated viruses and compositions (e.g., pharmaceutical compositions) containing them are within the scope of the present invention. The herpes virus particles can be partially purified from the cell or substantially purified (e.g., following a purification process, the herpes virus particles can constitute at least 85% (e.g., 90, 95, 99% or more) of the purified product. The compositions include cell-based and cell-free compositions. For example, the composition can include a host cell transduced with any of the altered herpes viruses described herein. The cell can be a mammalian cell (e.g., a human cell) and, with respect to cell type, can be any somatic cell susceptible to infection (e.g., a neuron or fibroblast). As noted, cells containing modified artificial chromosomes and/or altered herpes viruses that have packaged them can be arrayed, and such cellular arrays are within the scope of the present invention.

Methods of isolating herpes viruses from cells are known in the art and those methods can be applied to isolate the altered herpes viruses described herein. For example, the isolation methods can include lysing particle-containing cells; clearing or reducing the cellular debris; and applying the cleared remainder to a sucrose density gradient (particles come to reside at the interface). Purification can also be achieved by affinity chromatography. For example, one can immobilize an antibody or a fragment thereof (e.g., a single chain antibody that may be humanized) that recognizes a protein on the herpes virion (e.g., an env protein). The antibody can be immobilized on a column or other solid support. Once immobilized, the antibody can be exposed to a sample containing altered herpes viruses under conditions in which the antibody can specifically bind the particles. After the remainder of the sample is washed away, the antibody-virus interaction can be broken (e.g., the complex can be cleaved with a protease (e.g., an endopeptidase, a viral protease, or a combination thereof). Preferably, no protein is cleaved from the altered herpes virus.

Methods of identifying biologically active proteins: Other methods of the invention include methods of determining whether a protein alters the physiology of a cell affected by a birth defect. The protein can be a full-length or naturally occurring protein or a fragment or other mutant thereof (which may or may not retain biological activity). The methods can be carried out by (a) providing a cell; (b) exposing the cell to a herpes virus that includes a modified artificial chromosome having a sequence that encodes the protein; and (c) determining whether the protein alters the physiology of the cell. Preferably, the cell is exposed to the herpes virus for a time and under conditions in which the herpes virus transduces the cell and the nucleic acid sequence once carried by the artificial chromosome (the transgene or sequence of interest) is expressed as a protein within the cell. The cell can be any type of cell infectable by the altered herpes virus. For example, the cell can be a mammalian cell (e.g., a human cell). More specifically, the cell can be a neuron, a fibroblast, a blood cell, a hepatocyte, a keratinocyte, a melanocyte, a glial cell, an endocrine cell, an epithelial cell, a muscle cell, a bone cell, a prostate cell, a testicular cell, or a germ cell. The cell can also be diseased (e.g., malignant) and/or obtained at any developmental stage or at any stage of differentiation from a patient diagnosed as having a genetic defect or birth defect.

Methods of identifying therapeutic agents: Other methods of the invention include methods of identifying a candidate therapeutic agent by: (a) providing a cell; (b) exposing the cell to (i) the candidate therapeutic agent and (ii) a herpes virus comprising a modified artificial chromosome having a sequence of interest that encodes a protein; and (c) determining whether the candidate therapeutic agent affects the way in which the protein alters the physiology of the cell. Preferably, the cell is exposed to the herpes virus for a time and under conditions in which the herpes virus transduces the cell and a nucleic acid sequence of interest carried by the artificial chromosome is expressed as a protein within the cell. The candidate therapeutic agent can be applied before the cell is exposed to the altered herpes virus, simultaneously with (or in close sequence with) the application of the altered herpes virus, or after the virus has transduced the cell. The candidate therapeutic agent can be essentially any type of therapeutic agent, including a small molecule, a nucleic acid, or a protein (e.g., a protein described herein or an antibody that functions as an agonist or antagonist of a protein described herein), and the modified artificial chromosome can be any of those described herein. Similarly, the nucleic acid sequence of interest can be a genomic sequence or a cDNA sequence (e.g., a genomic human sequence or a human cDNA sequence or a sequence of a pathogen such as a virus, bacterium, fungus, parasite, or prion). Where nucleic acids are tested as therapeutic agents, those nucleic acids can mediate RNAi or may be more traditional antisense oligonucleotides. The nucleic acids can also encode functional proteins. Small molecules can be any organic or inorganic molecule, including those available in compound libraries, many of which are publicly or commercially available.

Methods of delivering therapeutic agents to a patient: Where the therapeutic agent is a protein, the altered herpes viruses described herein can be used to deliver that protein to a cell in vivo or in cell culture. The therapeutic agent can be one that is discovered in the screening methods of the present invention or a protein presently known or suspected of being therapeutic for a given disorder (i.e., the altered herpes viruses of the present invention can be used to deliver previously identified therapeutic proteins). Accordingly, the invention features methods of identifying a therapeutic protein, whether by using a screening method described herein or by surveying information within the public domain, and delivering that therapeutic protein to a cell in vivo or in cell culture. The protein can be delivered by exposing the cell to an altered herpes virus that expresses the protein for a time and under conditions that permit the virus to transducer the cell. In other embodiments, once the therapeutic protein is identified (by, for example, the screening process described above), it can be delivered to a patient by other vehicles. For example, it can be expressed by another viral vector (e.g., a retrovirus) or another type of vector (e.g., a plasmid).

In the event altered herpes viruses are introduced into cells in culture, the host cells can then be administered to patients. The cells administered may have been obtained initially from a patient and subsequently placed in culture; the administration can be of an autologous cell. However, the invention is not so limited. The cell can be any of a wide variety of types, so long as it is permissive for herpes virus propagation and compatible with the patient being treated (i.e., so long as the cell does not induce unacceptable side effects). As noted above, cells can be exposed to an altered herpes virus in combination with a vector that expresses an enzyme (e.g., a transposase) that facilitates chromosomal integration of the transgene carried by the modified artificial chromosome. Such an enzyme can be used when the cells are intended for administration to a patient, and cells (e.g., isolated cells or cells found ex vivo) and cell-based compositions (e.g., pharmaceutical compositions) bearing chromosomally integrated transgenes, originally carried by, for example, an artificial chromosome, are within the scope of the invention. We note, however, that the transgene may also be present episomally within a cell.

Generally, The patient may have any of a wide variety of diseases or conditions. For example, the patient can have an infectious disease. These patients may have been, or may become, infected with a wide variety of agents (including viruses such as a human immunodeficiency virus, human papilloma virus, herpes simplex virus, influenza virus, a pox virus, Ebola virus, bacteria (including eubacteria and archaea), such as Escherichia (e.g., E. coli) a Staphylococcus, Streptococcus, Campylobacter (e.g., C. jejuni), Listeria (e.g., L. monocytogenes), Salmonella, Shigella, or Bacillus (e.g., B. anthracis), a parasite, a mycoplasma, or an unconventional infectious agent such as a prior protein). The patient may also have, or be at risk for developing, a cancer (e.g., a leukemia or lymphoma) or other cellular proliferative disorder (e.g., a benign growth). Patients diagnosed as having a neurological deficit (e.g., a cognitive defect, motor disorder (including paralysis or parenthesis) or a sensory loss (e.g., an impaired sense of hearing, taste, smell, or sight), or a neurological disease (e.g., Parkinson's disease, Alzheimer's disease, or Huntington's disease) are also amenable to treatment. Other patients include those having a disease or condition that results from a genetic defect (e.g., cystic fibrosis) or birth injury (e.g., brain impairment due to oxygen deprivation). A patient having a disorder can be a patient diagnosed as having that disorder. Accordingly, a patient can be treated after they have been diagnosed as having a cancer, an infectious disease, or a neurological disorder, etc. . . . Similarly, since certain agents of the present invention can be formulated as vaccines, patients can be treated before they have developed the cancer, infectious disease, neurological disorder, or the like. Thus, “treatment” encompasses prophylactic treatment. For example, patients who have experienced a loss of hearing can be treated at any time, including before the loss occurs. For example, altered herpes viruses carrying a therapeutic transgene can be administered before the patient is exposed to some agent, such as a chemotherapeutic agent or industrial hazard, that may damage their hearing.

In all instances where a full-length protein can be used in the methods of the invention, a biologically active fragment or other mutant thereof can also be used. It follows that nucleic acid sequences that encode such biologically active fragments or mutants (e.g., proteins that are mutant by virtue of including one or more amino acid substitutions or additions) can also be used. These nucleic acid and protein variants can be used in methods for making a composition described herein (e.g., a modified artificial chromosome or altered herpes virus); in methods for screening for therapeutic agents; in methods for making pharmaceutical compositions; or in methods for administering the agents or compositions.

Kits: Kits that can be used to generate modified artificial chromosomes and/or altered herpes viruses as well as kits that can be used to screen for drug targets and therapeutic agents in the context of a birth defect are also within the scope of the present invention. For example, the invention features a kit that includes a targeting vector described herein and, optionally, an artificial chromosome that contains a nucleic acid sequence of interest. Alternatively, the kit can include a herpes virus amplicon particle including a transgene that, upon expression of RNA or a polypeptides, ameliorates a sign or symptom associated with a birth defect. The kits can also contain a composition (e.g., a physiologically acceptable composition) that contains such chromosomes or viruses. Alternatively, or in addition, the kits can contain host cells (e.g., prokaryotic host cells that include, or can include, a modified artificial chromosome or eukaryotic cells that include an altered herpes virus). Other kits can include one or more of the components useful in generating modified artificial chromosomes or altered herpes viruses. For example, a kit can include an enzyme to facilitate recombineering, a host cell, a helper virus, and/or a modified artificial chromosome. Alternatively, or in addition, the kits may include an enzyme, or a vector that encodes an enzyme, that mediates integration of the transgene carried by the modified artificial chromosome into the genome of a host cell. Where the kits are intended to aid screening assays, they may include cellular arrays and reagents for assessing physiological function. For example, the kits can include one or more reagents to assess the effect of a transgene on a cellular process (e.g., cell survival, the rate of cell division, differentiation potential, or regenerative activity). Any of the kits can also include instructions for use. The instructions can be conveyed by a variety of media (e.g., print, audiotape, videotape, CD, DVD, and the like). The compositions of the kits can be packaged in sterile form.

### EXAMPLES

### Example 1

### Neuronal Precursor-Restricted Transduction via in Utero CNS

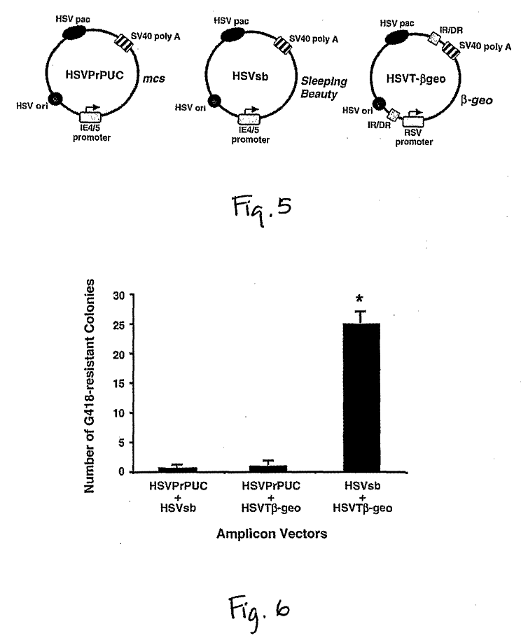

Gene Delivery of a Novel Bipartite HSVAmplicon/Transposase Hybrid Vector The ability of an HSV amplicon vector to deliver a transposable transcription unit for preferential expression in cells of the CNS was examined using a two-vector approach. To carry out this study, we constructed two vectors: one containing an SV40 promoter-driven β-galactosidase-neomycin (βgeo) fusion transgene flanked by the Sleeping Beauty (SB) inverted/direct repeats (HSVT0-βgeo), and a second containing the SB transposase gene transcriptionally driven by the HSV immediate-early 4/5 gene promoter (HSVsb) (see FIG. 5). We employed a two-vector strategy to preclude transposition events occurring when packaging the amplicon vectors using a modified helper virus-free methodology. Co-delivery of these vectors to the brains of E14.5 C57BL/6 mouse embryos resulted in the birth of viable neonates, integration of the transposable element from HSVT-βgeo, and extended transgene expression duration (at least 90 days) when compared to embryos transduced with HSVT-βgeo and empty vector control, HSVPrPUC.

A. In Vitro Characterization of the New Integration-Competent HSV Amplicon Vector

To determine if cotransduction with two amplicon vectors would result in enhanced integration in mitotically active cells, we initiated studies in HSV-susceptible baby hamster kidney (BHK) cells. We transduced BHK cultures with equivalent virion numbers of HSVsb+HSVPrPUC (empty vector control; FIG. 5), HSVT-hgeo+HSVPrPUC, or HSVT-hgeo+HSVsb. We placed the cultures under G418 selection, stained resistant colonies expressing the hgeo transgenon using X-gal histochemistry, and enumerated them. Cotransduction of HSVsb or HSVT-hgeo with the HSVPrPUC empty vector control amplicon resulted in very few G418-resistant, lacZ+ colonies (FIG. 6). By contrast, cotransduction of HSVsb with HSVT-hgeo greatly increased the number of colonies (˜25-fold), indicating that an HSV amplicon-delivered transgenon was stably maintained and expressed when briefly provided the transposase expressed from HSVsb. Percentages of G418-resistant colonies arising from transduced BHK cells ranged from 10 to 15% (data not shown). We did not measure the expression kinetics of HSVsb directly, but our previous studies showed that other IE4/5-driven transgenes exhibit the greatest gene product expression levels between 24 and 48 h posttransduction (Jin et al., Hum. Gene Ther. 7:2015-24, 1996; W. J. Bowers and H. J. Federoff, unpublished observations). This finding underscores the ability of transiently expressed SB to mobilize and catalyze transposition effectively.

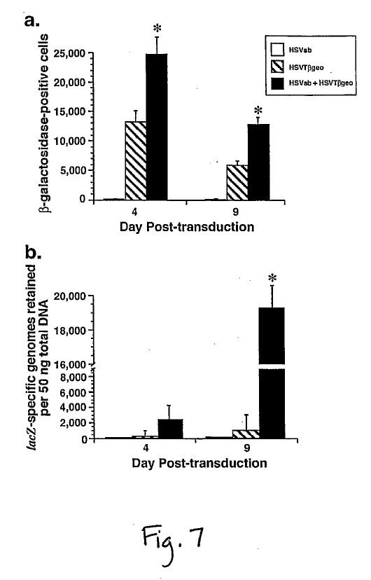

The observations in actively dividing BHK cells led to the evaluation of the new bipartite amplicon platform in primary murine cortical cultures to determine whether transposition of the amplicon-bearing transgene unit would occur in neural cells. We established primary cultures using B27 medium, which resulted in cultures of nearly exclusively neuronal cell types with minimal glial contamination. We incubated primary cortical cultures with equivalent numbers of transducing virions of HSVsb, HSVT-hgeo, or both vectors on day 5 in vitro (DIV 5). We analyzed treated cultures for X-gal histochemistry and real-time quantitative PCR analysis for the transgenon DNA segment. Enumeration of X-gal-positive cells in each of the treatment groups indicated that cultures receiving both test amplicons exhibited enhanced numbers of transgene-expressing cells on days 4 and 9 (FIG. 7A). Separate immunocytochemical analysis of cultures indicated that both neurons and rare glia expressed the βgeo transgene (data not shown). When we harvested DNA from transduced cultures using a method favoring the purification of chromosomal DNA (Beerman et al., Mech. Dev. 42:59-65, 1993), the cultures receiving both HSVsb and HSVT-hgeo amplicons exhibited an increased number of lacZ sequence targets over time (FIG. 7B). Taken in aggregate, these data suggested that in the presence of HSVsb the transgenon segment of the HSVT-hgeo amplicon had associated with host cell genomic DNA and that resulted in appreciably enhanced gene expression compared to cultures transduced with HSVT-hgeo alone. Transgenon expression resulting from the HSVsb+HSVT-βgeo treatment appeared to diminish slightly as a function of time (FIGS. 7A and 7B). Levels of βgeo gene product at early assay time points (<14 days posttransduction) likely represent the sum of amplicon episome- and integrant-mediated expression. Diminution of expression at later time points could be the result of a host-mediated cis repression phenomenon that has been shown to occur to vectors harboring the RSV promoter (see, e.g., Yeh et al., J. Biol. Chem. 270:15815-20, 1995; Laker et al., J. Virol. 72:339-48). In addition, subsets of transgenons could be localized to regions of chromatin that are undergoing progressive heterochromatin formation (see, e.g., Boyer et al., J. Immunol. 159:3383-90, 1997).

B. Assessment of Transgenon Integration Sites

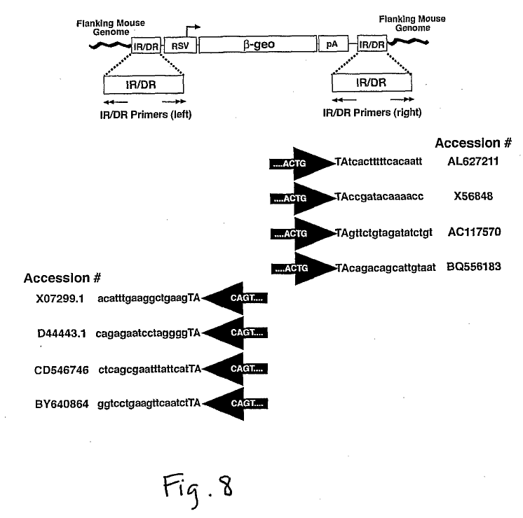

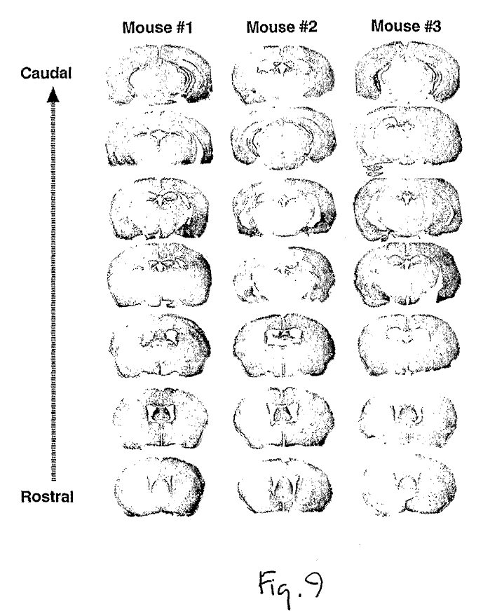

To assess definitively the occurrence of SB-mediated integration into the genome of mouse primary cortical cultures, we employed inverse PCR (see, e.g., Luo et al., Hum. Gene Ther. 6:421-430, 1995). On day 9 posttransduction, we subjected high-molecular-weight DNA isolated from primary cultures transduced with both HSVsb and HSVT-hgeo to three rounds of nested PCR. We sequenced the resultant integration junction PCR products and analyzed the identity of novel flanking nucleotide sequences. Several different flanking murine genomic sequence were identified by BLAST searches (FIG. 8). Integration sites detected included sequence homologous to the malate dehydrogenase gene (Accession No. X07299.1), dexamethasone-induced product gene (Accession No. D44443.1), an EST (Accession No.CD546746), and a RIKEN clone (Accession No. BY640864). We also found integration sites within unannotated regions of mouse chromosomes 4 (Accession No. AL627211) and 9(Accession No. AC117570). From the multiple sequences analyzed, there did not appear to be a sequence-specific preference for integration of the T-hgeo transgenon within the genome.

C. In Utero Delivery of the Integration-Competent HSV Amplicon to the Embryonic Mouse Brain

The new integrating system was evaluated in utero by gene transfer to the developing CNS of embryonic day 14.5 (E14.5) mice. Embryos transduced in utero with a 1:1 ratio of HSVPrPUC+HSVT-βgeo or HSVsb+HSVT-βgeo (FIG. 5) were re-introduced into the uterus, allowed to reach full term and then placed with Swiss Webster foster mothers. Brains were harvested, sectioned and processed from immunocytochemistry for β-galactosidase alone or in combination with cellular markers on postnatal day 90 (P90; approximately 97 days post-transduction).

Co-transduction of E14.5 mouse embryos with HSVsb and HSVT-βgeo led to widespread βgalactosidase expression (FIG. 9). The expression patterns arising from the βgeo transgenon (transposable transgene) from representative coronal brain sections from each of three mice receiving HSVsb+HSVT-βgeo are depicted in the rostrocaudal axis (coordinates ranging from +1.0 mm to −3.0 mm relative to Bregma). Transgenon expression was most pronounced in the subventicular zone, septofimbrial region, dentate gyrus, hippocampus, and the primary and secondary motor cortices. Histological assessment revealed no evidence of brain architecture alterations or cellular abnormalities at 90 days of age. Genomic DNA harvested from the hippocampus of two 21 day-old mice co-injected at E14.5 with HSVsb and HSVT-βgeo was subjected to inverse PCR analysis to determine amplicon vector/mouse genome junctions. A small number of inverse PCR products were derived from the brain tissue of these mice. Sequence analysis of the four isolated junction regions indicated the integration sites were located within unannotated regions of mouse chromosomes 3 (Accession #AC124190), 8 (Accession#AC145211), 11 (Accession #AL596456), and 12 (Accession #AC131991).

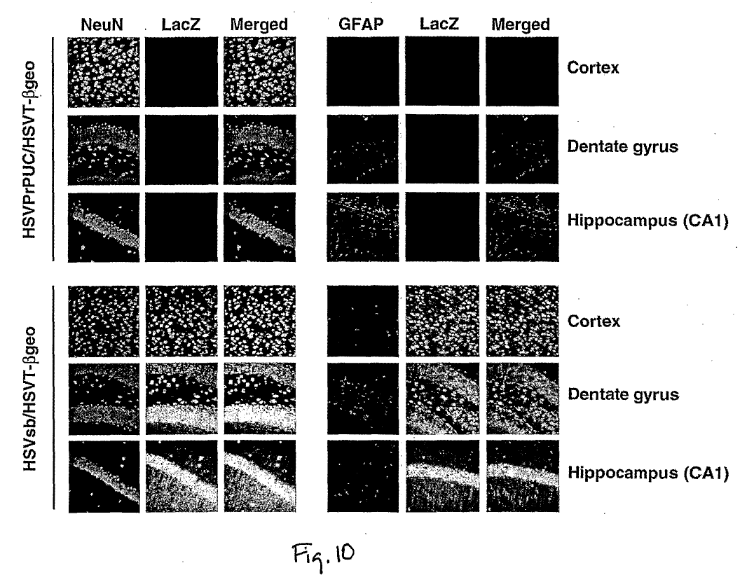

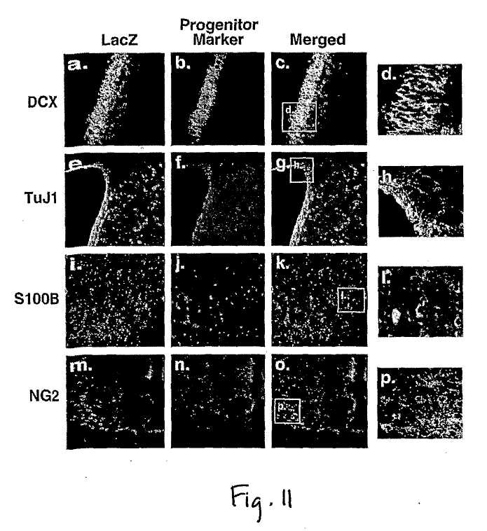

Microscopic examination of brain sections corresponding to the cortex, dentate gyrus, and the CA1 region of the hippocampus (FIG. 10) revealed many transgenon-expression β-galactosidase-positive cells (apparent in green when viewed in a color photograph). Intracranial injection of E14.5 C57BL/6 mouse embryos with HSVPrPUC and HSVT-βgeo (n=8) resulted in no detectable expression of β-galactosidase at the time of sacrifice (97 days post-transduction) in any of the regions of the brains analyzed (FIG. 10, upper panels). Conversely, all mice (n=8) receiving intracranial inoculations of both HSVsb and HSVT-βgeo at E14.5 showed consistent neuronal expression of the T-βgeo transgenon at 97 days following vector delivery (FIG.-10, lower panels). Few GFAP+iglia were noted to be β-galactosidase labeled. β-galactosidase expression was strikingly robust within the soma and processes of neurons residing in the cortex and CA1 pyramidal layer of the hippocampus. NeuN/LacZ dual positivity was also observed within regions of the dentate gyrus enriched in GABAergic interneurons (FIG. 10, lower panels) and within the hilar region and granule neurons of the infrapyramidal blade.

The overwhelming numbers of lacZ-positive mature neurons detected throughout the brains of 90-day-old HSVsb/HSVT-βgeo-injected mice strongly imply that embryonic intraventricular infusion of these vectors led to selective transduction of, integration within, and/or specific RSV promoter-driven transgenon expression in neural precursor cells populating the subventricular zone. The possibility, however, does exist that embryonic infusion led to transduction of migrating or mature neurons via inadvertent intraparenchymal injection during the surgical procedure. If a stem-like cell pool was initially transduced, one would expect to find neural-restricted precursor cells that express β-galactosidase in the adult mouse brain. We subsequently performed fluorescence immunocytochemistry on adjacent brain sections using antibodies specific for the following committed precursor cell markers: doublecortin (DCX), which is expressed by migrating neuroblasts; class III β-tubulin (TuJ1), which is found on immature neurons; the astroglial marker S100b; and the immature oligodendrocyte marker NG2. Immunocytochemical analysis of brain sections corresponding to periventricular regions (FIG. 11) revealed numerous lacZ-positive cells (which appear green when viewed in a color photograph), which were colabeled (red; see merged channel, yellow) with the neuronal precursor markers DCX and TuJ1, but not the S100b or NG2 surface markers. This observation indicates embryonic codelivery of HSVsb and HSVT-hgeo selectively transduced neuronally committed precursor cells, which remain transgenon-positive at 97 days posttransduction.

The utility of the HSV amplicon vector platform is greatly extended by the development of this integration-capable iteration. Its simplicity, relating to its minimal requirements of one effector protein and small flanking cis DNA elements, makes this approach an attractive alternative to pursue novel therapeutic modalities for prenatally detectable diseases that affect the nervous system. In addition, the amplicon could be engineered to transiently coexpress host factors known to participate in SB-mediated transposition to enhance integration into desired regions. One such factor is the highly-conserved DNA-bending protein, HMGB1 (see, e.g., Zayed et al., Nucleic Acids Res., 31: 2313-22, 2002).

Moreover, the use of cell-type-specific promoters could drive transgenon expression in defined regions of the brain, thereby adding a layer of regulation that may be important for specific indications. Although we observed very few transduced glial cells, the selection of neuronal promoters, which are transactivated following cell cycle withdrawal, would mitigate the potential for inadvertent activation of a dormant proto-oncogene within a cell type with mitogenic capability. Further, the type of cells transduced can be altered potentially by modifying the tropism of the HSV amplicon virion. Grandi and colleagues recently molecularly modified glycoprotein C to bind specifically to an engineered cellular receptor and, in doing so, effectively altered the tropism of the virus (Grandi et al., Mol. Ther. 9:419-27, 2004). This approach could be theoretically extended to target specific subsets of cells in the developing embryo.

Directing transgenon integration to a “safe” chromosomal site would be desirable in a clinical application. The SB transposon integrates exclusively into a TA dinucleotide motif that is duplicated as a result of transposition (see, e.g., Izsvak et al., Mol. Ther. 9:147-56, 2004), but does not appear to have genome specificity. Additionally, SB-mediated transpositions are precise events that do not result in chromosomal recombination or deletion (see, e.g., Izsvak et al.), thereby distinguishing this form of gene mobilization from that of other viral vector systems (i.e., rAAV and retrovirus/lentivirus). The adaptation of this transposition paradigm to the HSV amplicon may provide a means to promote region-specific integration. The large genomic capacity of the amplicon allows for the incorporation of segments of DNA homology that may increase the frequency of integration into a desired chromosomal region. In addition, the amplicon could be engineered to transiently coexpress host factors known to participate in SB-mediated transposition to enhance integration into desired regions, e.g., HMGB1 (see, e.g., Zayed et al., Nucleic Acids Res., 31: 2313-22, 2002).

The utility of the HSV amplicon vector platform is greatly extended by the development of integration-capable iterations, including the HSV/AAV hybrids and the currently described SB-based form. The simplicity of the latter, relating to its minimal requirements of one effector protein and small flanking cis DNA elements, makes this approach an attractive alternative to pursue novel therapeutic modalities for prenatally detectable diseases that affect the nervous system. In addition, the stable maintenance of the integration-competent amplicon following embryonic administration enables a vast number of new applications for studying cell fate determination and the function of gene products in precursor biology and their differentiated postmitotic types. Through further engineering, the safety profile of this vector system will be enhanced without compromising its intrinsic efficiency.

Cell culture: Baby hamster kidney (BHK) cells were maintained as described by Lu et al. (Human Gene Ther. 6:421-430, 1995). The NIH-3T3 mouse fibroblast cell line was originally obtained from American Type Culture Collection and maintained in Dulbecco's modified Eagle medium (DMEM) supplemented with 10% fetal bovine serum (FBS), 100 units/ml penicillin, and 100 μg/ml streptomycin. Primary cortical neurons were harvested from E15 mice and were prepared according to published methods (Yant et al., Nature Genetics 25:35-41, 2000). Cortices were dissociated initially by trypsinization (0.25% trypsin/EDTA) for 15 minutes at 37° C. and washed twice with HBSS containing Ca⁺ and Mg²⁺. Cells were mechanically dissociated further using a serologic pipette and resuspended in serum-free Neurobasal® plating medium containing 0.5 mM L-glutamine, 3.7 μg/ml L-glutamate and 2% B-27 supplement (Life Technologies, Gaithersburg, Md.). Cultures were maintained at 37° C. in a 6% CO₂ environment. Cultures were transduced helper virus-free HSV amplicon stocks at a multiplicity of infection (MOI) of 0.5 on Day 4 in vitro (DIV).

Amplicon construction: The SB transposase encoding sequence was removed from the pCMV-SB plasmid (Yant et al., Nature Genetics 25:35-41, 2000; kindly provided by Dr. M. Kay) by XhoI/SalI digestion and cloned into the SalI site of pHSVPrPUC to create pHSVsb (Geller et al., Proc. Natl. Acad. Sci. USA 87:8950-8954, 1990). The integration-competent transcription cassette from pT-βgeo (also provided by Dr. M. Kay) was removed using KpnI and VspI, blunted, and cloned into the blunted HindIII site of pHSV minOriSmc amplicon to create pHSVT-βgeo (Yant et al., Nature Genetics 25:35-41, 2000). In a subset of experiments the pHSVPrPUC amplicon was employed as an empty vector control and was previously described (Geller et al., Proc. Natl. Acad. Sci. USA 87:8950-8954, 1990).

Helper virus-free HSV amplicon packaging: Amplicon vectors were packaged as previously described (Bowers et al., Gene Ther. 8:111-120, 2001). Viral pellets were resuspended in 100 μl PBS and stored at −80° C. until use. Vectors were titered as described previously (Bowers et al., Mol. Ther. 1:294-299, 2000).

Real-time quantitative PCR analyses. To isolate total DNA for quantitation of amplicon genomes in transduced cells or brain tissue, isolates were lysed in 100 mM potassium phosphate (pH 7.8) and 0.2% Triton X-100. An equal volume of 2× Digestion Buffer (0.2 M NaCl, 20 mM Tris-Cl (pH 8.0), 50 mM EDTA, 0.5% SDS, 0.2 mg/ml proteinase K) was added to the lysate and the sample was incubated at 56° C. for 4 hours. Samples were processed further by one phenol:chloroform, one chloroform extraction, and a final ethanol precipitation. Total DNA was quantitated and 25 ng of total DNA was analyzed in a PE7700 quantitative PCR reaction using a designed lacZ-, or β-lactamase transposase gene-specific primer/probe combination multiplexed with an 18S rRNA-specific primer/probe set. The lacZ probe sequence was 5′-6FAM-ACCCCGTACGTCTTCCCGAGCG-TAMRA-3′; the lacZ sense primer sequence was 5′-GGGATCTGCCATTGTCAGACAT-3′; and the lacZ antisense primer sequence was 5′-TGGTGTFFFCCATAATTCAA-3′. The β-lactamase probe sequence was 5′-6FAM-CAGGACCACTTCTGCGCTCGGC-TAMRA-3′; the β-lactamase antisense primer sequence was 5′-CGGCTCCAGATTTATCAGCCAAT-3′. The 18S rRNA probe sequence was 5′-MAX-TGCTGGCACCAGACTTGCCCTC-TAMRA-3′; the 18S sense primer sequence was 5′-CGGCTACCACATCCAAGGAA-3′; and the 18S antisense primer sequence was 5′-GCTGGAATTACCGCGGCT-3′.

Analysis of integrated vector sequences: Inverse PCR was utilized for analysis of junction fragments as previously described by Luo et al. (Proc. Natl. Acad. Sci. USA., 95:10769-10773, 1998) using the identical three sets of nested primers that were designed for both the left (IR/DR-L) and right ends of the ITR (IR/DR-R). Briefly, genomic DNA was purified from amplicon-transduced primary neuronal cultures at Day 9 post-transduction or from the brains of mice receiving HSVsb and HSVT-βgeo in utero using a previously described method with a phenol:chloroform extraction step (Beermann et al., Mech. Dev. 42:59-65, 1993), digested with Sau3AI, and ligated with T4 DNA ligase. Samples were subsequently subjected to three rounds of PCR using the nested primer sets. Amplified products arising from the third PCR reaction were ligated into the pGEMT-Easy (Promega) or pCR2.1-TOPO (Invitrogen, Carlsbad, Calif.) cloning vector and sequenced using the dye terminator method.

In utero gene delivery: Under the surgical plane of anesthesia (Avertin, 0.5 mg/g), the maternal abdomen of post-coitum 14.5 C57BL/6 mice were shaved and prepped with proviodine scrub (Operand, Bradford, Conn.). A laparotomy was performed and the uterus was gently exteriorized onto a sterile disposable drape. The embryo was visualized using an Olympus SZ60 dissection microscope (Olympus, Japan). Injection of fetuses was performed with a Borosil micropipette needle (FHC, Inc., Bowdoinham, Me.) with a diameter of 1.0 mm, created with a Narishige PB-7 needle puller (Narishige International USA, Inc, New York, N.Y.), and then ground to a 30° angle with a Narishige EG-44 microgrinder (Narishige International USA, Inc, New York, N.Y.). Two microliters of HSVsb or the control amplicon HSVPrPuc was mixed, at a ratio of 1:1 (2×10⁴ total volume, 2×10⁴ total transducing units), with HSVT-βgeo, and delivered intracranially to the mouse embryo, using an IM300 Programmable Microinjector (Narishige International USA, Inc, New York, N.Y.). Efforts were made to restrict infusion to the ventricle, but the possibility exists that a subset of viral particles was delivered to the parenchyma. (Herpes virus amplicon particles carrying other transgenes can be microinjected or otherwise application to other tissues, such as muscle.) The uterus was returned to the abdominal cavity and the abdominal wall was closed using coated VICRYL (polyglactin 910) sutures (Ethicon, Somerville, N.J.), and the outer incision was closed with silk sutures (Tyco healthcare, VIIIe St. Laurent, Quebec). A layer of triple antibiotic gel (Fougera, Melville, N.Y.) was applied over the incision site, and the mouse monitored for breathing and reflexive movements until it regained consciousness. Throughout surgery the mouse was maintained on a 36° C. water pad circulated by a T-pump heat therapy system (Gaymar, Orchard Park N.Y.). Upon recovery the mouse was given 0.00325 mg/kg Buprenorphine hydrochloride (Reckitt Benkisen Healthcare Ltd., Hull, England) subcutaneously and returned to its cage. The mouse was monitored for weight gain and movement, and 5% lidocaine (Fougera, Melville, N.Y.) was applied to the incision site twice a day until it gave birth. Once born, the pups were placed in the care of a Swiss Webster foster mother (Jackson Laboratories, Bar Harbor, Me.), allowed to develop 28 or 90 days old, sacrificed, and processed for integration site analysis and immunocytochemical analyses (n=8 per treatment group).

Tissue preparation and immunocytochemistry: In utero-injected E14.5 embryos were sacrificed at 90 days post partum (97 days post injection) for immunocytochemistry. Following administration of anesthesia, a catheter was placed into the left ventricle of the heart, and intracardiac perfusion was initiated with 10 ml of heparinized saline (5,000 U/L saline) followed by 30 ml of chilled 4% PFA in saline. Brains were excised and post-fixed for 4-8 hours in 4% PFA at 4° C. Subsequently, brains were cryoprotected in a series of sucrose solutions with a final solution consisting of a 30% sucrose concentration (w/v) in PBS. Thirty-micron serial sections were cut using a sliding microtome (Micron/Zeiss, Thornwood, N.Y.) and stored in a cryoprotective solution (30% sucrose (w/v), 30% ethylene glycol in 0.1 M phosphate buffer (pH 7.2)) at −20° C. until processed for immuncytochemistry. Upon removal of cryoprotectant, sections were placed into Costar net wells (VWR, Springfield, N.J.) and incubated for two hours in 0.1 M phosphate buffered saline (PBS) (pH 7.6). Sections were permeabilized in 0.1 M PBS and 0.1% Triton-X-100 for five minutes at 25° C. Non-specific binding sites were blocked using 0.1 M PBS, 10% normal goat serum and 0.1% Triton-X-100 for one hour at 25° C. Double immunocytochemistry was performed using anti-β-galactosidase, rabbit IgG Fraction (1:2000; Biodesign, Saco Me.), with either mouse anti-Neuronal Nuclei (NeuN) monoclonal antibody (1:200; Chemicon International; Temecula, Calif.), or an anti-Glial Fibrillary Acid Protein (GFAP)-cy3 conjugate monoclonal antibody clone G-A-5 (1:2000; Sigma, St. Louis, Mo.). Sections were incubated for 24 hours at 4° C. with primary antibodies diluted in 0.1 M PBS, 1% normal goat serum and 0.1% Triton-X-100. After rinsing in 0.1 M PBS (5×5 minutes), fluorescent secondary antibodies (Alexa 488 anti-rabbit IgG (H+L; 1:200; Molecular Probes, Eugene, Oreg.) and Rhodamine Red™-X-conjugated AffiniPure goat anti-mouse IgG (H+L) (1:200; Jackson Immuno Research Laboratories Inc., West Grove, Pa.), diluted in 0.1 M PBS plus 1% normal goat serum and 0.1% Triton-X-100, were added to the sections and incubated for two hours at 25° C. The sections were rinsed in 0.1 M PBS, mounted on glass slides with Mowiol, and visualized using a confocal laser scanning microscope (FV 300, Olympus, Melville, N.Y.) at 20× or 60×. For diaminobenzidine (DAB) labeling of cells for detection of β-galactosidase, secondary goat anti-rabbit HRP-conjugated (1:1000; Jackson Immuno Research Laboratories Inc., West Grove, Pa.) was used. The DAB precipitant was then developed for 4-7 minutes using the DAB Peroxide substrate kit (Vector Laboratories, Burlingame, Calif.). The sections subsequently were rinsed in 0.1 M PBS, mounted on glass slides, cleared with Histoclear™ (National Diagnostics, Atlanta, Ga.), and coverslipped using Cytoseal™ mounting medium (Stephens Scientific, Riverdale, N.J.). Photomicrographs were digitally acquired using an Olympus Provis AX70 microscope (Olympus America Inc., Melville, N.Y.) and Spot RT camera (Diagnostic Instruments Inc., Sterling Heights, Mich.) at 1.25×, 40× or 100× magnification.

### Example 2

### A Recombineering Protocol for Modification of a BAC Vector Placed into E. coli Strain EL250 Containing Defective Lambda Prophage

To prepare competent cells, we streaked E. coli strain EL250 cells from a glycerol stock stored at −80° C. onto an LB plate. From an individual colony that arose on the plate, we inoculated a 50 ml LB culture (250 ml Ehrlenmyer flask), and grew the liquid culture at 32° C. overnight in a shaking incubator (2000 rpm). We removed the culture and chilled the flask in a slurry of ice and water, gently shaking by hand to chill the cells quickly. We transferred 10 ml of culture to a 15 ml conical tube and centrifuged it at 3500 rpm in a Beckman SLA-1500 rotor for 5 min at 4° C. (stopping with no brake). After pouring off the supernatant, we gently resuspended the cells in 10 ml of sterile double distilled water (ddH₂O). The resuspended cells were centrifuged at 3500 rpm in a Beckman SLA-1500 rotor for 5 minutes at 4° C. (and allowed to slow with no brake). The supernatant was again removed and the cells were resuspended in 1 ml of sterile ddH₂O. The washing step was repeated twice more, for a total of three washes with 1 ml of sterile ddH₂O. After a final centrifuge, we resuspended the cells in 80 μl of sterile ddH₂O to obtain cells resuspended in a final volume of 100 μl.

To introduce the targeting vector, we combined 10-100 ng of vector DNA with 50 μl of competent cells in a 1.5 ml microfuge tube and chilled the mixture on ice for 5 minutes. We then pipetted the cells into a prechilled 1 mm cuvette (BioRad) and electroporated them with 1.75 kV and 186 Ohms. We added 450 μl of SOC media to the cuvette and transferred the entire contents to 1.5 ml microfuge tube, which was incubated at 32° C. for 30 minutes. We then plated the cells using selective media (Ram^{r} for BAC).

To prepare a linear targeting vector, a selected circular targeting vector can be digested and the products separated by gel electrophoresis. The desired fragment can then be cut out of the gel and purified. Alternatively, a linear nucleic acid to be used for recombineering can be generated by PCR. The desired product can then be isolated from a gel (e.g., an acrylamide gel).

The RED genes used for recombination are under the control of a heat inducible promoter. The strains are briefly heated to 42° C. to allow expression and then chilled to reduce activity until the introduction of the PCR cassette through electroporation.

To induce RED genes, we inoculated 5 ml of LB medium containing a selective antibiotic with strain EL250 cells containing the vector to be modified. The cells were grown overnight at 32° C., and one ml of the overnight culture was inoculated into 50 ml of fresh media (in a 500 ml flask). The culture was grown at 32° C. until the OD₆₀₀ equaled 0.5-0.8. We then transferred 10 ml of culture to a 125 ml flask and place it in a 42° C. waterbath for 15 minutes. The flask was then moved to a slurry of ice and water and swirled gently to quickly chill the cells. We then transferred the culture to a 15 ml conical tube and centrifuged it at 3500 rpm in a Beckman SLA-1500 rotor for 8 minutes at 4° C. (the centrifuge slowed with no brake). We poured off the supernatant and gently resuspended the cells in 1 ml of sterile ddH₂O. The cells were pelleted again in a 4° C. microfuge at maximum speed for 20 seconds. We repeated the washing step twice more, for a total of three washes in 1 ml of sterile ddH₂O. After the final spin, we resuspended the cells in 100 μl of sterile ddH₂O.

For the final electroporation, we placed 50-100 ng of a PCR-generated cassette and 50 μl of competent cells in a 1.5 ml microfuge tube and chilled the tube on ice for 5 minutes. We pipetted the cells into a prechilled 1 mm curvette (BioRad) and electroporated them using 1.75 kV and 186 Ohms. Following electroporation, we added 950 μl of SOC media to the cuvette and transferred the entire contents to a 1.5 ml microfuge tube, which we incubated at 32° C. for 30 minutes. The cells were then plated on selective medium (Ram^{r} plates for BAC).

### Example 3

### Amplicon BAC Engineering for Discovery of New Molecules Involved in Neural Regeneration and Repair

A major obstacle in the treatment of traumatic injuries to the brain or spinal cord is the incapacity of neurons in the adult central nervous system (CNS) to regenerate damaged axons. One important factor attributed to this regenerative failure is the growth inhibitory environment encountered by injured axons. It is well established that adult CNS neurons possess the intrinsic machinery to grow axons, and when provided with a favorable environment, may extend axons over long distances. Multiple lines of evidence point to adult CNS myelin as a major barrier for axonal growth and regeneration. Several myelin-derived inhibitors have been identified, including myelin associated-glycoprotein (MAG), Nogo-A, oligodendrocyte-myelin glycoprotein (OMgp) and most recently, Semaphorin 4D. In addition, chondroitin sulfate proteoglycans and secreted semaphorins associated with the glial scar contribute to the growth inhibitor environment of injured CNS tissue (Filbin, Nature Rev. Neurosci. 4:703-713, 2003).

The recent identification of a neuronal surface receptor for Nogo66, called NgR1 (former NgR), provides for the first time mechanistic insights into Nogo function. NgR1, a member of a leucine-rich repeat (LRR) family, is linked to the cell surface through a glycosylphosphatidyl inositol (GPI) anchor and forms a heteromeric complex with p75NTR and LINGO-1 to signal inhibition across the neuronal cell membrane. Although Nogo, MAG and OMgp lack sequence homologies, they all bind to the NgR1 and recent data suggest that the myelin inhibitory proteins Nogo, MAG, and OMgp all signal growth inhibition through a NgR1/p75NTR/LINGO-1 receptor complex (Mi et al., Nature Neurosci. 7:221-228, 2004).

Considerable progress has been made in identifying myelin inhibitory proteins and their receptors. While growing evidence suggests that RhoA is a key mediator of growth inhibition, the molecular events leading to growth cone collapse and a net loss of actin polymerization at the leading edge of an (injured) axon are not well defined. Recent work suggests that conventional isoforms of PKC are upstream of RhoA and that neuronal expression of dominant negative forms of conventional PKCs attenuates myelin inhibition (Sivasankaran et al., Nature Neurosci. 7:261-268, 2004).

Perhaps most interestingly from a therapeutic point of view are recent findings showing that adult mammalian neurons can be “primed” by pre-exposure to neurotrophins (BDNF or NGF). Priming leads to a transcription/translation-dependent silencing of the classical RhoA inhibitory signaling pathway, which allows adult neurons to extend processes in the presence of myelin inhibitory proteins. Priming is mediated by activation of the cAMP-PKA pathway, which leads to CREB-mediated gene expression. One of the down-stream products of priming has been identified as arginase-1 (Cai et al., Neuron 35:711-719, 2002). Consistent with a key role in ‘primed neurons’, ectopic expression of arginase-1 allows neurons to grow process extensions on a MAG/myelin substrate.

Here, we propose to use an in vitro neurite outgrowth assay on myelin substrate combined with HSV-vector mediated gene transfer to screen for gene products that attenuate or overcome myelin-mediated inhibition of neurite outgrowth. As a control experiment, we propose to introduce arginase-1 into postnatal cerebellar granule neurons (CGNs) using HSV-mediated gene transfer. Neurite length of arginase-1 expressing CGCs will be quantified and compared to CGCs infected with a control HSV-vector carrying a reporter transgene.

Arginase-1 positive control: As a positive control for the proposed screen, HSV-BAC mediated neuronal expression of arginase-1, an enzyme previously shown to allow neurons to grow in the presence of myelin inhibitory proteins, will be used to demonstrate the feasibility of our approach.

In a first series of experiments, we will demonstrate expression of arginase-1 from cells infected with an HSV vector carrying a retrofitted arginase-1 BAC(HSV-BAC/arginase-1; a modified artificial chromosome). Expression will be monitored by Western blotting of HSV-BAC/arginase-1 infected COS-7 cells and immunocytochemistry of neurons infected with HSV-BAC/arginase-1. BAC clones carrying the arginase-1 gene will be ordered from BACPAC and retrofitted with HSV amplicon sequences (for details see below). For immunoblotting and immunocytochemistry, we will use a polyclonal anti-arginase-1 antibody (Abcam ab2111). To confirm that HSV-BAC/arginase-1 infected neurons overexpress arginase-1, we will double stain for arginase-1 and the neuron-specific marker TuJ1 using the anti-class III tubulin antibody (TuJ1; Promega).

Next, we will address whether HSV-BAC/arginase-1 mediated overexpression of arginase-1 in DRG neurons overcomes myelin mediated inhibition of neurite outgrowth. This will allow us to calibrate the neurite outgrowth assay (i.e., to establish myelin concentrations that allow for a large shift in neurite length following overexpression of arginase-1).

Next, we will determine the dilution of HSV-BAC/arginase-1 that still leads to a significant change in neurite length in our functional assay. Serial dilutions of HSV-BAC/arginase-1 with a control HSV-lacZ vector will be used to infect primary neurons. This will allow us to determine the complexity of viral pools optimal for the proposed screen and give an estimate of how many viral pools will have to be screened to cover the entire genome at least twice.

To show that we can identify arginase-1 from a complex viral pool containing HSV-BAC/arginase-1, the original pool will be divided into sub-pools and tested in our functional assay until single HSV clones are obtained. The BAC DNA of the identified HSV will be sequenced directly, or subcloned to demonstrate it contains the arginase-1 gene.

Preparation of HSV-BAC amplicon library: the Herpes Simplex Virus (HSV) amplicon vector has proven useful for highly efficient gene transfer into many mammalian cell types. As noted above, the amplicon is a circular DNA requiring only two cis elements from a herpes virus for production in virions. These are the “a” sequence, which is required for packaging, and an HSV origin (ori) of replication. These two sequences are sufficient to confer onto a DNA plasmid the ability to be replicated, cleaved, and inserted into an HSV viral envelope. By transducing cells with amplicons and amplicon-associated vectors, we are able to measure functional outcomes associated with the expression of transferred genes such as myelin responsiveness of primary neurons.

We will use a herpes amplicon library carrying murine BACs to identify genes important for CNS regeneration and repair. We propose to construct a library of HSV-BACs each containing a unique segment of chromosomal DNA from a human. Specifically, we propose to use BAC engineering techniques to generate this library. These HSV-BACs will be packaged into amplicon virions and used, for example, for functional genomic studies.

Generation of HSV-BAC amplicon vector: A BAC will be selected. In making the selection, we may consider its suitability for library construction, which is improved where primer sites for subsequent sequencing are included and backbone sequences divergent from HSV BAC are used in packaging to reduce the risk of recombination. Next, a cassette containing the HSV origins(s) and packaging site and selectable markers (Kan^{r} and dsRED) will be inserted into the BAC using recombineering within several sites of the backbone. Each vector will be tested to determine which construct results in the highest titer of infectious particles.

Construction of HSV-BAC amplicon library (a library of modified artificial chromosomes): We intend to outsource the construction of a human BAC library. We expect a service provider to provide ˜3 times the coverage of the human genome, resulting in ˜9,000 clones with insert size of ˜100 kb. We will ask that these clones be arrayed as single clones on microtiter plates and combined to make pools and super pools.

Packaging of the BAC amplicon library: Our group as well as several others have described helper virus-free packaging methods. We can convert an amplicon DNA, in this case our retrofitted BAC library (containing modified artificial chromosomes), into virus by the co-transfection of a separate BAC carrying the HSV replication and packaging sequences. Utilized in this way, we will prepare a population of virions that should represent, in a one-step packaging process, the complete collection of genomic BAC sequences. These will be characterized in a variety of different assays to make certain that there has been no significant skewing of the population and they will be utilized in cell culture studies to make sure that they are fully effective and capable of transduction.

Identification of Proteins Affecting Growth of Neuronal Processes: To visualize the axon growth inhibitory activity of CNS myelin, a number of robust and well-established cell culture assays may be used. Typically, crude or partially purified myelin fractions are spotted on polylysine and used as a substrate to culture dissociated neurons. A variation of this assay used in our laboratory is to adsorb cryosections of brain and/or spinal cord tissue directly onto glass coverslips in multiwell culture plates. Postnatal neurons are then plated on tissue sections to assess fiber length and number on CNS white matter and gray matter substrates. A major strength of this assay is that it allows us to directly compare fiber growth on gray (permissive) and white (non-permissive) matter. Furthermore, the concentration of tissue-associated inhibitors is likely to be comparable to that encountered by regenerating CNS fibers. This is particularly relevant when dealing with strategies designed to overcome myelin inhibition and promote axonal regeneration in a CNS environment.

Preparations of myelin inhibitory substrate: Myelin inhibitory proteins will be isolated from adult rat spinal cord. Briefly, spinal cords (10 g) from adult rat will be dissected, homogenized, and extracted in ice-cold CHAPS buffer (60 mM CHAPS, 100 mM Tris pH 8.0, 10 mM EDTA, 2% protease inhibitor cocktail (Sigma)). Extracted proteins will be separated from cell debris by two high speed spins (Beckman table top ultracentrifuge; 200,000×g 1 hour each). The clear supernatant will be fractionated over a mono-Q ion exchange column using a BioRad (DuoFlow) FPLC using a linear 0-1M NaCl gradient. Fractions eluting between 0.25-0.5 M NaCl will be pooled, dialyzed and used as an inhibitory substrate for neurite outgrowth.

Primary neuronal cultures: Standard procedures will be used for primary neuronal cultures. For neurite outgrowth assays we use routinely rat P7-P10 cerebellar granule cells and adult rat DRGs.

### Example 4

### Production of a Helper Virus-Free Amplicon Particle

As noted above, HSV-based amplicon particles are attractive gene delivery tools, and they are particularly well suited for delivering gene products to neurons (e.g. neurons in the central nervous system) because they are easy to manipulate, can carry large transgenes, and are naturally neurotropic (Geller and Breakefield, Science 241:1667-1669, 1988; Spaete and Frenkel, Cell 30:305-310, 1982; Federoff et al., Proc. Natl. Acad. Sci. USA 89:1636-1640, 1992; Federoff in Cells: A Laboratory Manual, Spector et al., Eds., Cold Spring Harbor Press, Cold Spring Harbor, N.Y., 1997; Frenkel et al., in Eucaryotic Viral Vectors, Gluzman, Ed., Cold Spring Harbor Press, Cold Spring Harbor, N.Y., 1982). Efforts to bring this vector system into the clinical arena to treat neurodegenerative disease have been hampered by potential cytotoxicites that are associated with traditional methods of virus packaging. This problem involves the co-packaging of helper virus that encodes cytotoxic and immunogenic viral proteins. Newer methods of packaging have been developed that result in helper virus-free amplicon stocks (Fraefel et al., J. Virol. 70:7190-7197, 1996; Stavropoulos and Strathdee, J. Virol. 72:7137-7143, 1998; see also U.S. Pat. Nos. 5,851,826 and 5,998,208). Stocks prepared by these methods, however, are typically low titer (<10⁵ expression units/ml), allowing for only modest scale experimentation, primarily in vitro. Such low titers make large animal studies difficult, if not impossible. Present helper virus-free packaging strategies lead to not only lower amplicon titers, but also to stocks that exhibit a high frequency of pseudotransduction events when used to infect a variety of cell types.

Optimal propagation of wild-type HSV virions requires orderly progression of α, β and γ gene transcription following infection of a host cell. This is achieved by delivery of co-packaged proteins, carried by the virion, that help co-opt the cell's transcription machinery and transactivation of viral α gene promoters. This information is fundamental to the development of our helper virus-free system. Helper virus-based packaging involves superinfection of an amplicon DNA-transfected monolayer of packaging cells with a replication-defective helper virus. The helper virus genome, as in the case of wild-type HSV, is delivered to the cell in a complex with co-packaged proteins, including VP16 and virion host shutoff (vhs). The HSV vhs protein functions to inhibit the expression of genes in infected cells via destabilization of both viral and host mRNAs. Because vhs plays such a vital role in establishing the HSV replicative cycle and is a potential structural protein, we hypothesized that its presence during amplicon packaging accounted for the higher titers obtained with helper virus-based packaging systems. VP16 is another co-packaged protein that resides in the helper virus nucleocapsid and is responsible for activating transcription of HSV immediate-early genes to initiate the cascade of lytic cycle-related viral protein expression.

In contrast to helper virus-based packaging systems, helper virus-free systems involve co-transfection of naked DNA forms of either an HSV genome-encoding cosmid set or BAC reagent with an amplicon vector (e.g., a plasmid). Thus, the HSV genome gains access to the cell without co-packaged vhs or VP16. The initiation and temporal progression of HSV gene expression is, we speculated, not optimal for production of packaged amplicon vectors due to the absence of these important HSV proteins. To test our hypothesis—that the efficiency of amplicon packaging would be increased by introducing vhs and/or VP16 during the initial phase of virus propagation—we included a vhs-encoding DNA segment in the packaging protocol as a co-transfection reagent. In some instances, packaging cells were “pre-loaded” with VP16 to mimic its presence during helper virus-mediated amplicon packaging. As shown below, these modifications led to a 30- to 50-fold enhancement of packaged amplicon vector titers, nearly approximatig titers obtained using helper virus-based traditional approaches. In addition, the viral stocks failed to exhibit the pseudotransduction phenomenon. These improvements make large-scale in vivo applications much more likely. The methods used to make a helper virus-free amplicon particles are described first, followed by a description of the results obtained.

Cell culture: Baby hamster kidney (BHK) cells were maintained as described by Lu et al. (Human Gene Ther. 6:421-430, 1995). NIH 3T3 cells were originally obtained from the American Type Culture Collection and were maintained in Dulbecco's modified Eagle medium (DMED) supplemented with 10% fetal bovine serum, penicillin, and streptomycin.

Plasmid construction: The HSVPrPUC/CMVegfp amplicon plasmid was constructed by cloning the 0.8-kb cytomegalovirus (CMV) immediate early promoter and 0.7-kb enhanced green fluorescent protein cDNA (Clontech, Inc.) into the BamHI restriction enzyme site of the pHSVPrPUC amplicon vector (Geller et al., Proc. Natl. Acad. Sci. USA 87:8950-8954, 1990). A 3.5 kb HpaI/HindIII fragment encompassing the UL41 (vhs) open reading frame and its 5′ and 3′ transcriptional regulatory elements was removed from cos 56 (Cunningham and Davison, Virol. 197:116-124, 1993) and cloned into pBSKSII (Stratagene, Inc.) to create pBSKS(vhs). For construction of pGRE₅vp16, the VP16 coding sequence was amplified by PCR from pBAC-V2 using gene-specific oligonucleotides that possess EcoRI (5′-CGGAATTCCGCAGGTTTTGTAATGTATGTGCTCGT-3′ (SEQ ID NO:2) and HindIII (5′-CTCCGAAGCTTAAGCCCGATATCGTCTTTCCCGTATCA-3′ (SEQ ID NO:3)) restriction enzyme sequences that facilitate cloning into the pGRE₅-2 vector (Mader and White, Proc. Natl. Acad. Sci. USA 90:5603-5607, 1993).

Helper virus-free Amplicon Packaging: On the day prior to transfection, 2×10⁶ BHK cells were seeded on a 60-mm culture dish and incubated overnight at 37° C. The following procedures were followed for cosmid-based packaging. The day of transfection, 250 μl Opti-MEM (Gibco-BRL, Bethesda, Md.), 0.4 μg of each of five cosmid DNAs (kindly provided by Dr. A. Geller, and 0.5 μg amplicon vector DNA, with or without varying amounts of pBSKS(vhs) plasmid DNA were combined in a sterile polypropylene tube (Fraefel et al., J. Virol. 70:7190-7197, 1996). The following procedures were followed for BAC-based packaging. 250 μl Opti-MEM (Gibco-BRL, Bethesda, Md.), 3.5 μg of pBAC-V2 DNA (kindly provided by Dr. C. Strathdee, and 0.5 μg amplicon vector DNA, with or without varying amounts of pBSKS(vhs) plasmid DNA were combined in a sterile polypropylene tube (Stavropoulos and Strathdee, J. Virol. 72:7137-7143, 1998). The protocol for both cosmid- and BAC-based packaging was identical from the following step forward. Ten microliters of Lipofectamine Plus™ reagent (Gibco-BRL) were added over a 30-second period to the DNA mix and allowed to incubate at room temperature for 20 minutes. In a separate tube, 15 μl Lipofectamine (Gibco-BRL) were mixed with 250 μl Opti-MEM. Following the 20 minute incubation, the contents of the two tubes were combined over a one-minute period and then incubated for an additional 20 minutes at room temperature. During the second incubation, the medium in the seeded 60 mm dish was removed and replaced with 2 ml Opti-MEM. The transfection mix was added to the flask and allowed to incubate at 37° C. for five hours. The transfection mix was then diluted with an equal volume of DMEM plus 20% FBS, 2% penicillin/streptomycin, and 2 mM hexamethylene bis-acetamide (HMBA), and incubated overnight at 34° C. The following day, medium was removed and replaced with DMEM plus 10% FBS, 1% penicillin/streptomycin, and 2 mM HMBA. The packaging flask was incubated an additional three days and virus was harvested and stored at −80° C. until purification. Viral preparations were subsequently thawed, sonicated, and clarified by centrifugation (3000×g for 20 minutes). Viral samples were stored at −80° C. until use.

For concentrated viral stocks, viral preparations were subsequently thawed, sonicated, clarified by centrifugation, and concentrated by ultracentrifugation through a 30% sucrose cushion (Geschwind et al., Providing pharmacological access to the brain in Methods in Neuroscience, Conn, Ed., Academic Press, Orlando, Fla., 1994). Viral pellets were resuspended in 100 μl PBS and stored at −80° C. until use. For packaging experiments examining the effect of VP16 on amplicon titers, the cells plated for packaging were first allowed to adhere to the 60 mm culture dish for 5 hours and subsequently transfected with pGRE₅vp16 using the Lipofectamine reagent as described above. Following a five-hour incubation, the transfection mix was removed, complete medium (DMEM plus 10% FBS, 1% penicillin/streptomycin) was added, and the cultures were incubated at 37° C. until the packaging co-transfection step the next day.

Viral titering: Amplicon titers were determined by counting the number of cells expressing enhanced green fluorescent protein (HSVPrPUC/CMVegfp amplicon) or β-galactosidase (HSVlac amplicon). Briefly, 10 μl of concentrated amplicon stock was incubated with confluent monolayers (2×10⁵ expressing particles) of NIH 3T3 cells plated on glass coverslips. Following a 48-hr incubation, cells were either fixed with 4% paraformaldehyde for 15 min at RT and mounted in Mowiol for fluorescence microscopy (eGFP visualization), or fixed with 1% glutaraldehyde and processed for X-gal histochemistry to detect the lacZ transgene product. Fluorescent or X-gal-stained cells were enumerated, expression titer calculated, and represented as either green-forming units per ml (gfu/ml) or blue-forming units per ml (bfu/ml), respectively.

TaqMan Quantitative PCR System: To isolate total DNA for quantitation of amplicon genomes in packaged stocks, virions were lysed in 100-mM potassium phosphate pH 7.8 and 0.2% Triton X-100. Two micrograms of genomic carrier DNA was added to each sample. An equal volume of 2× Digestion Buffer (0.2 M NaCl, 20 mM Tris-Cl pH 8.0, 50 mM EDTA, 0.5% SDS, 0.2 mg/ml proteinase K) was added to the lysate and the sample was incubated at 56° C. for 4 hrs. Samples were processed further by one phenol:chloroform, one chloroform extraction, and a final ethanol precipitation. Total DNA was quantitated and 50 ng of DNA was analyzed in a PE7700 quantitative PCR reaction using a designed lacz-specific primer/probe combination multiplexed with an 18S rRNA-specific primer/probe set. The lacZ probe sequence was 5′-6FAM-ACCCCGTACGTCTTCCCGAGCG-TAMRA-3′ (SEQ ID NO:4); the lacZ sense primer sequence was 5′-GGGATCTGCCATTGTCAGACAT-3′ (SEQ ID NO:5); and the lacZ antisense primer sequence was 5′-TGGTGTGGGCCATAATTCAA-3′ (SEQ ID NO:6). The 18S rRNA probe sequence was 5′-JOE-TGCTGGCACCAGACTTGCCCTC-TAMRA-3′ (SEQ ID NO:7); the 18S sense primer sequence was 5′-CGGCTACCACATCCAAGGAA-3′ (SEQ ID NO:8); and the 18S antisense primer sequence was 5′-GCTGGAATTACCGCGGCT-3′ (SEQ ID NO:9).

Each 25-μl PCR sample contained 2.5 μl (50 ng) of purified DNA, 900 nM of each primer, 50 nM of each probe, and 12.5 μl of 2× Perkin-Elmer Master Mix. Following a 2-min 50° C. incubation and 2-min 95° C. denaturation step, the samples were subjected to 40 cycles of 95° C. for 15 sec. and 60° C. for 1 min. Fluorescent intensity of each sample was detected automatically during the cycles by the Perkin-Elmer Applied Biosystem Sequence Detector 7700 machine. Each PCR run included the following: no-template control samples, positive control samples consisting of either amplicon DNA (for lacZ) or cellular genomic DNA (for 18S rRNA), and standard curve dilution series (for lacZ and 18S). Following the PCR run, “real-time” data were analyzed using Perkin-Elmer Sequence Detector Software version 1.6.3 and the standard curves. Precise quantities of starting template were determined for each titering sample and results were expressed as numbers of vector genomes per ml of original viral stock.

Western blot analysis: BHK cell monolayers (2×10⁶ cells) transfected with varying packaging components were lysed with RIPA buffer (150 mM NaCl, 1% NP-40, 0.5% DOC, 0.5% SDS, and 50 mM Tris-Cl, pH 8). Equal amounts of protein were electrophoretically separated on a 10% SDS-PAGE gel and transferred to a PVDF membrane. The resultant blot was incubated with an anti-VP16 monoclonal antibody (Chemicon, Inc.), and specific VP16 immunoreactive band visualized using an alkaline phosphatase-based chemiluminescent detection kit (ECL).

Cytotoxicity Assays: The effect of BAC-packaged HSVlac stocks prepared in the presence or absence of VP16 and/or vhs on cell viability was determined using a lactate dehydrogenase (LDH) release-based assay (Promega Corp., Madison, Wis.). Equivalent expression units of virus from each packaging sample were used to transduce 5×10³ NIH 3T3 cells in 96-well flat-bottomed culture dishes. Quantitation of LDH release was performed according to manufacturer's instructions. Viability data were represented as normalized cell viability index.

Stereotactic injections: Mice were anesthetized with Avertin at a dose of 0.6 ml per 25 g body weight. After positioning in an ASI murine stereotactic apparatus, the skull was exposed via a midline incision, and burr holes were drilled over the following coordinates (bregma, +0.5 mm; lateral −2.0 mm; and deep, −3.0 mm) to target infections to the striatum. A 33 GA steel needle was gradually advanced to the desired depth, and 3 μl (equivalent in vitro titer) HSVPrPUC/CMVegfp virus was infused via a microprocessor-controlled pump over 10 minutes (UltraMicroPump, World Precision Instruments, Sarasota Springs, Fla.). The injector unit was mounted on a precision small animal stereotaxic frame (ASI Instruments, Warren, Mich.) micromanipulator at a 90° angle using a mount for the injector. Viral injections were performed at a constant rate of 300 nl/min. The needle was removed slowly over an additional 10-minute period.

Tissue preparation and GFP visualization: Infected mice were anesthetized four days later, a catheter was placed into the left ventricle, and intracardiac perfusion was initiated with 10 ml of heparinized saline (5,000 U/L saline) followed by 60 ml of chilled 4% PFA. Brains were extracted and postfixed for 1-2 hours in 4% PFA at 4° C. Subsequently, brains were cryoprotected in a series of sucrose solutions with a final solution consisting of a 30% sucrose concentration (w/v) in PBS. Forty micron serial sections were cut on a sliding microtome (Micron/Zeiss, Thomwood, N.Y.) and stored in a cryoprotective solution (30% sucrose (w/v), 30% ethylene glycol in 0.1 M phosphate buffer (pH 7.2)) at −20° C. until processed for GFP visualization. Sections were placed into Costar net wells (VWR, Springfield, N.J.) and incubated for 2 hrs in 0.1 M Tris buffered saline (TBS) (pH 7.6). Upon removal of cryoprotectant, two additional 10 min washes in 0.1 M TBS with 0.25% Triton X-100 (Sigma, St. Louis, Mo.) were performed. Sections were mounted with a fine paint brush onto subbed slides, allowed to air dry, and mounted with an aqueous mounting media, Mowiol. GFP-positive cells were visualized with a fluorescent microscope (Axioskop, Zeiss, Thornwood, N.Y.) utilizing a FITC cube (Chroma Filters, Brattleboro, Vt.). All images used for morphological analyses were digitally acquired with a 3-chip color CCD camera at 200× magnification (DXC-9000, Sony, Montvale, N.J.).

Morphological analyses: Cell counts were performed on digital images acquired within 24 hrs of mounting. At the time of tissue processing coronal slices were stored serially in three separate compartments. All compartments were processed for cell counting and GFP(+) cell numbers reflect cell counts throughout the entire injection site. All spatial measurements were acquired using an image analysis program (Image-Pro Plus, Silver Spring, Md.) at a final magnification of 200×. Every section was analyzed using identical parameters in three different planes of focus throughout the section to prevent repeated scoring of GFP(+) cells. Each field was analyzed by a computer macro to count cells based on the following criteria: object area, image intensity (fluorescent signal) and plane of focus. Only cells in which the cell body was unequivocally GFP(+) and nucleus clearly defined were counted. Every section that contained a GFP(+) cell was counted. In addition, a watershed separation technique was applied to every plane of focus in each field to delineate overlapping cell bodies. The watershed method is an algorithm that is designed to erode objects until they disappear, then dilates them again such that they do not touch.

Statistical Analyses: Statistical analyses were carried out using one-way analyses of variance (ANOVA) with plasmid construct as the between-group variable. Two-way repeated measure analyses of variance (RMANOVA) were carried out using plasmid construct as the between-group variable and time interval as a within-group variable.

Results: Prior to the methods described herein, widespread use of helper virus-free HSV particles has been hampered by helper virus-mediated cytotoxicity associated with traditionally packaged amplicon stocks or by the low titers obtained from helper virus-free production methods. Helper virus-free methods of packaging hold the most promise as resultant stocks exhibit little or no cytotoxicity. As shown here, modifications to such packaging strategies could be made to increase viral titers.

We utilized both cosmid- and BAC-based methods of helper virus-free packaging previously described (Fraefel et al., J. Virol 70:719-7197, 1996; Stavropoulos and Strathdee, J. Virol. 72:7137-7143, 1998; and Saeki et al., Hum. Gene Ther. 9:2787-2794, 1998). The low titers observed for helper virus-free methods may be a result of the sub-optimal state of the HSV genome at the beginning of amplicon production, as the genome is without co-packaged viral regulators vhs and VP16. To determine if introduction of vhs into the packaging scheme could increase amplicon titers and quality, we cloned a genomic segment of the UL41 gene into pBluescript and added this plasmid (pBSKS(vhs)) to the co-transfection protocols to provide vhs in trans. The genomic copy of UL41 contained the transcriptional regulatory region and flanking cis elements believed to confer native UL41 gene expression during packaging. When pBSKS(vhs) was added to the packaging protocols for production of a β-galactosidase (lacZ)-expressing amplicon (HSVlac), a maximum of 10-fold enhanced amplicon expression titers was observed for both cosmid- and BAC-based strategies. As observed previously, the expression titers for HSVlac virus produced by the BAC-based method were approximately 500- to 1000-fold higher than stocks produced using the modified cosmid set. Even though titers were disparate between the differently prepared stocks, the effect of additionally expressed vhs on amplicon titers was analogous.

The punctate appearance of reporter gene product (pseudotransduction), a phenomenon associated with first-generation helper virus-free stocks, was substantially diminished in vitro when vhs was included in BAC-based packaging of a β-galactosidase-expressing (HSVlac) or an enhanced green fluorescent (GFP)-expressing virus (HSVPrPUC/CMVegfp). Pseudotransduction was not observed, as well, for cosmid-packaged amplicon stocks prepared in the presence of vhs. To assess the ability of the improved amplicon stocks to mediate gene delivery in vivo, BAC-packaged HSVPrPUC/CMVegfp virus prepared in the absence or presence of pBSKS(vhs) was injected stereotactically into the striata of C57BL/6 mice (see above). Four days following infection, animals were sacrificed and analyzed for GFP-positive cells present in the striatum. The numbers of cells transduced by HSVPrPUC/CMVegfp prepared in the presence of vhs were significantly higher than in animals injected with stocks produced in the absence of vhs. In fact, it was difficult to definitively identify GFP-positive cells in animals transduced with vhs(−) amplicon stocks.

The mechanism by which vhs expression resulted in higher apparent amplicon titers in helper virus-free packaging could be attributed to one or several properties of vhs. The UL41 gene product is a component of the viral tegument and could be implicated in structural integrity, and its absence could account for the appearance of punctate gene product material following transduction. For example, the viral particles may be unstable as a consequence of lacking vhs. Thus, physical conditions, such as repeated freeze-thaw cycles or long-term storage, may have led to inactivation or destruction of vhs-lacking virions at a faster rate than those containing vhs.

The stability of HSVPrPUC/CMVegfp packaged via the BAC method in the presence or absence of vhs was analyzed initially with a series of incubations at typically used experimental temperatures. Viral aliquots from prepared stocks of HSVPrPUC/CMVegfp were incubated at 4, 22, or 37° C. for periods up to three hours. Virus recovered at time points 0, 30, 60, 120, and 180 minutes were analyzed for their respective expression titer on NIH 3T3 cells. The rates of decline in viable amplicon particles, as judged by their ability to infect and express GFP, did not differ significantly between the vhs(+) and vhs(−) stocks. Another condition that packaged amplicons encounter during experimental manipulation is freeze-thaw cycling. Repetitive freezing and thawing of virus stocks is known to diminish numbers of viable particles, and potentially the absence of vhs in the tegument of BAC-packaged amplicons leads to sensitivity to freeze fracture. To test this possibility, viral aliquots were exposed to a series of four freeze-thaw cycles. Following each cycle, samples were removed and titered for GFP expression on NIH 3T3 cells as described previously. At the conclusion of the fourth freeze-thaw cycle, the vhs(−) HSVPrPUC/CMVegfp stock exhibited a 10-fold diminution in expression titers as opposed to only a 2-fold decrease for vhs(+) stocks. This observation suggests that not only do vhs(+) stocks have increased expression titers, but the virions are more stable when exposed to temperature extremes, as determined by repetitive freeze-thaw cycling.

The native HSV genome enters the host cell with several viral proteins besides vhs, including the strong transcriptional activator VP16. Once within the cell, VP16 interacts with cellular transcription factors and HSV genome to initiate immediate-early gene transcription. Under helper virus-free conditions, transcriptional initiation of immediate-early gene expression from the HSV genome may not occur optimally, thus leading to lower than expected titers. To address this issue, a VP16 expression construct was introduced into packaging cells prior to cosmid/BAC, amplicon, and pBSKS(vhs) DNAs, and resultant amplicon titers were measured. To achieve regulated expression a glucocorticoid-controlled VP16 expression vector was used (pGRE₅vp16).

The pGRE₅vp16 vector was introduced into the packaging cells 24 hours prior to transfection of the regular packaging DNAs. HSVlac was packaged in the presence or absence of vhs and/or VP16 and resultant amplicon stocks were assessed for expression titer. Some packaging cultures received 100-nM dexamethasone at the time of pGRE₅vp 16 transfection to strongly induce VP16 expression; others received no dexamethasone. Introduction ofpGRE₅vp16 in an uninduced (basal levels) or induced state (100 nM dexamethasone) had no effect on HSVlac titers when vhs was absent from the cosmid- or BAC-based protocol. In the presence of vhs, addition of pGRE₅vp 16 led to either a two- or five-fold enhancement of expression titers over those of stocks packaged with only vhs (cosmid- and BAC-derived stocks). The effect of “uninduced” pGRE₅vp 16 on expression titers suggested that VP16 expression was occurring in the absence of dexamethasone. To examine this, Western blot analysis with a VP16-specific monoclonal antibody was performed using lysates prepared from BHK cells transfected with the various packaging components. Cultures transfected with pGRE₅vp16/BAC/pBSKS(vhs) in the absence of dexamethasone did show VP16 levels intermediate to cultures transfected either with BAC alone (lowest) or those transfected with pGRE₅vp16/BAC/pBSKS(vhs) in the presence of 100 nM dexamethasone (highest) (FIG. 4C). There was no difference in level of pGRE₅vp16-mediated expression in the presence or absence of BAC, nor did dexamethasone treatment induce VP16 expression from the BAC.

VP16-mediated enhancement of packaged amplicon expression titers could be due to increased DNA replication and packaging of amplicon genomes. Conversely, the additional VP16 that is expressed via pGRE₅vp16 could be incorporated into virions and act by increasing vector-directed expression in transduced cells. To test the possibility that VP16 is acting by increasing replication in the packaging cells, concentrations of vector genomes in BAC-derived vector stocks were determined. HSVlac stocks produced in the presence or absence of vhs and/or VP16 were analyzed using a “real-time” quantitative PCR method. The concentration of vector genome was increased two-fold in stocks prepared in the presence of VP16 and this increase was unaffected by the presence of vhs.

There is a possibility that addition of viral proteins, like vhs and VP16, to the packaging process may lead to vector stocks that are inherently more cytotoxic. The amplicon stocks described above were examined for cytotoxicity using a lactate dehydrogenase (LDH) release-based cell viability assay. Packaged amplicon stocks were used to transduce NIH 3T3 cells and 48 hours following infection, viability of the cell monolayers was assessed by the LDH-release assay. Amplicon stocks produced in the presence of vhs and VP16 displayed less cytotoxicity on a per virion basis than stocks packaged using the previously published BAC-based protocol (Stavropoulos and Strathdee, supra).

Significance: Wild-type HSV virions contain multiple regulatory proteins that prepare an infected host cell for virus propagation. These virally encoded regulators, which are localized to the tegument and nucleocapsid, include vhs and VP16, respectively. The UL41 gene-encoded vhs protein exhibits an essential endoribonucleolytic cleavage activity during lytic growth that destabilizes both cellular and viral mRNA species (Smibert et al., J. Gen. Virol. 73:467-470, 1992). Vhs-mediated ribonucleolytic activity appears to prefer the 5′ ends of miRNAs over 3′ termini, and the activity is specific for mRNA, as vhs does not act upon ribosomal RNAs (Karr and Read, Virology 264:195-204, 1999). Vhs also serves a structural role in virus particle maturation as a component of the tegument. HSV isolates that possess disruptions in UL41 demonstrate abnormal regulation of IE gene transcription and significantly lower titers than wild-type HSV-1 (Read and Frenkel, J. Virol. 46:498-512, 1983), presumably due to the absence of vhs activity. Therefore, because vhs is essential for efficient production of viable wild-type HSV particles, it likely plays a similarly important role in packaging of HSV-1-derived amplicon vectors.

The term “pseudotransduction” refers to virion expression-independent transfer of biologically active vector-encoded gene product to target cells (Liu et al., J. Virol. 70:2497-2502, 1996; Alexander et al. Human Gene Ther. 8:1911-1920, 1997. This phenomenon was originally described with retrovirus and adeno-associated virus vector stocks and was shown to result in an overestimation of gene transfer efficiencies. β-galactosidase and alkaline phosphatase are two commonly expressed reporter proteins that have been implicated in pseudotransduction, presumably due to their relatively high enzymatic stability and sensitivity of their respective detection assays (Alexander et al., supra). Stocks of β-galactosidase expressing HSVlac and GFP-expressing HSVPrPUC/CMVegfp exhibited high levels of pseudotransduction when packaged in the absence of vhs. Upon addition of vhs to the previously described helper virus-free packaging protocols, a 10-fold increase in expression titers and concomitant decrease in pseudotransduction were observed in vitro.

Vhs-mediated enhancement of HSV amplicon packaging was even more evident when stocks were examined in vivo. GFP-expressing cells in animals transduced with vhs(+) stocks were several hundred-fold greater in number than in animals receiving vhs(−) stocks. This could have been due to differences in virion stability, where decreased particle stability could have led to release of co-packaged reporter gene product observed in the case of vhs(−) stocks. Additionally, the absence of vhs may have resulted in packaging of reporter gene product into particles that consist of only tegument and envelope (Rixon et al., J. Gen. Virol. 73:277-284, 1992). Release of co-packaged reporter gene product in either case could potentially activate a vigorous immune response in the CNS, resulting in much lower than expected numbers of vector-expressing cells.

Pre-loading of packaging cells with low levels of the potent HSV transcriptional activator VP16 led to a 2- to 5-fold additional increase in amplicon expression titers only in the presence of vhs for cosmid- and BAC-based packaging systems, respectively. This observation indicates the transactivation and structural functions of VP16 were not sufficient to increase viable viral particle production when vhs was absent, and most likely led to generation of incomplete virions containing amplicon genomes as detected by quantitative PCR. When vhs was present for viral assembly, however, VP16-mediated enhancement of genome replication led to higher numbers of viable particles formed. Quantitative PCR analysis of amplicon stocks produced in the presence of VP16 and vhs showed that viral genomes were increased only 2-fold while expression titers were increased 5-fold over stocks produced in the presence of vhs only. This result suggests that a portion of the effect related to VP16-mediated enhancement of genome replication while the additional ˜2-fold enhancement in expression titers may be attributed to the structural role of VP16. The effect of VP16 on expression titers was not specific to amplicons possessing the immediate-early 4/5 promoter of HSV, as amplicons with other promoters were packaged to similar titers in the presence of VP16 and vhs.

VP16 is a strong transactivator protein and structural component of the HSV virion (Post et al., Cell 24:555-565, 1981). VP16-mediated transcriptional activation occurs via interaction of VP16 and two cellular factors, Oct-1 (O'Hare and Goding, Cell 52:435-445, 1988; Preston et al., Cell 52:425-434, 1988; Stem et al., Nature 341:624-630, 1989) and HCF (Wilson et al., Cell 74:115-125, 1993; Xiao and Capone, Mol. Cell. Biol. 10:4974-4977, 1990) and subsequent binding of the complex to TAATGARAT elements found within HSV IE promoter regions (O'Hare, Semin. Virol. 4:145-155, 1993. This interaction results in robust up-regulation of IE gene expression. Neuronal splice-variants of the related Oct-2 transcription factor have been shown to block IE gene activation via binding to TAATGARAT elements (Lillycrop et al., Neuron 7:381-390, 1991) suggesting that cellular transcription factors may also play a role in limiting HSV lytic growth.

The levels of VP16 appear to be important in determining its effect on expression titers. Low, basal levels of VP16 (via uninduced pGRE₅vp16) present in the packaging cell prior to introduction of the packaging components induced the largest effect on amplicon expression titers. Conversely, higher expression of VP16 (via dexamethasone-induced pGRE₅vp16) did not enhance virus production to the same degree and may have, in fact, abrogated the process. The presence of glucocorticoids in the serum components of growth medium is the most likely reason for this low-level VP16 expression, as charcoal-stripped sera significantly reduces basal expression from this construct. Perhaps only a low level or short burst of VP16 is required to initiate IE gene transcription, but excessive VP16 leads to disruption of the temporal progression through the HSV lytic cycle, possibly via inhibition of vhs activity. Moreover, evidence has arisen to suggest vhs activity is downregulated by interaction with newly synthesized VP16 during the HSV lytic cycle, thereby allowing for accumulation of viral mRNAs after host transcripts have been degraded (Schmelter et al., J. Virol. 70:2124-2131, 1996; Smibert et al., J. Virol. 68:2333-2346, 1994; Lam et al., EMBO J. 15:2575-2581, 1996). Therefore, a delicate regulatory protein balance may be required to attain optimal infectious particle propagation. Additionally, the 100-nM dexamethasone treatment used to induce VP16 expression may have a deleterious effect on cellular gene activity and/or interfere with replication of the OriS-containing amplicon genome in packaging cells. High levels of dexamethasone have been shown previously to repress HSV-1 OriS-dependent replication by an unknown mechanism Hardwicke and Schaffer, J. Virol. 71:3580-3587, 1997). Inhibition of OriS-dependent replication does not appear to be responsible for our results, however, since quantitative PCR analysis of amplicon stocks produced in the presence and absence of dexamethasone indicated no change in genome content as a function of drug concentration. It is interesting to note that amplicon stocks were prepared in the presence of hexamethylene bisacetamide (HMBA). HMBA has been shown to compensate for the absence of VP16, thus leading to the transactivation of immediate early gene promoters (McFarlane et al., J. Gen. Virol. 73:285-292, 1992. In the absence of HMBA pre-loading a packaging cell with VP16 could impart an even more dramatic effect on titers.

Ectopic expression of vhs and VP16 did not lead to amplicon stocks that exhibited higher cytotoxicity than helper virus-free stocks prepared in the traditional manner when examined by an LDH-release assay. Stocks prepared by the various methods were equilibrated to identical expression titers prior to exposure to cells. The heightened cytotoxicity in stocks produced in the absence of vhs and/or VP16 may reflect that larger volumes of these stocks were required to obtain similar expression titers as the vhs/VP16-containing samples or the levels of defective particles in the former may be significantly higher. Contaminating cellular proteins that co-purify with the amplicon particles are most likely higher in concentration in the traditional stocks, and probably impart the higher toxicity profiles observed.

Abovis; Acebutolol; Acebutolol hydrochloride; Acemetacin; Acepreval; Acetaldehyde; Acetamide; 5-Acetamide-1,3,4-thiadiazole-2-sulfonamide; Acetazolamide sodium; Acetic acid methylnitrosaminomethyl ester; Acetohydroxamic acid; Acetonitrile; 3-(alpha-Acetonyl-para-nitrobenzyl)-4-hydroxy-coumarin; para-Acetophenetidide; 17-Acetoxy-19-nor-17-alpha-pregn-4-EN-20-YN-3-one; Acetoxyphenylmercury; Acetoxytriphenylstannane; 1-alpha-Acetylmethadol hydrochloride; Acetylsalicylic acid; Acetyltryptophan; Acid red 92; 4,-(9-Acridinylamino)methanesulphon-meta-anisidide; Acriflavin hydrochloride; Acrylic acid; Acrylonitrile; Actihaemyl; Actinomycin; Actinomycin C; Actinomycin D; Acyclovir; Acyclovir sodium salt; Adalat; 1-Adamantanamine hydrochloride; Adapin; Adenine; Adenosine-3,-(alpha-amino-para-methoxyhydrocinnamamido)-3,-deoxy-n,n-dimethyl; Adipic acid bis(2-ethylhexyl)ester;
Adipic acid dibutyl ester; Adipic acid di(2-hexyloxyethyl)ester; Adobiol; Adona trihydrate; 1-Adrenaline chloride; Adrenocorticotrophic hormone; Adriamycin; Aflatoxin; Aflatoxin B1; Afridol blue; Agent orange; Alclometasone dipropionate; Alcohol sulphate; Aldactazide; Aldecin; Aldimorph; Aldrin; alpha-Alkenesulfonic acid; Alkyl dimethylbenzyl ammonium chloride; 3-(Alkylamino)propionitrile; Alkylbenzenesulfonate; Allantoxanic acid, potassium salt; Alloxan; Allyl chloride; Allyl glucosinolate; Allyl isothiocyanate; 6-Allyl-6,7-dihydro-5h-dibenz(c,e) azepine phosphate; Allylestrenol; (4-Allyloxy-3-chlorophenyl)acetic acid; Alternariol; Alternariol monomethyl ether and alternariol (1:1); Alternariol-9-methyl ether; Aluminum aceglutamide; Aluminum chloride; Aluminum chloride hexahydrate; Aluminum lactate; Aluminium (III) nitrate, nonahydrate (1:3:9); Aluminium potassium sulfate, dodecahydrate; Ambroxol hydrochloride; Ametycin; Amfenac sodium monohydrate; Amicardine; N1-Amidinosulfanilamide; Amidoline; 5-((2-Aminoacetamido) methyl)-1-(4-chloro-2-(orthochlorobenzoyl)phenyl)-n,n-dimethyl-1H-S-triazole-3-carboxamide, hydrochloride, dehydrate; Aminoacetonitrile bisulfate; Aminoacetonitrile sulfate; 2-Aminobenzimidazole; 2-Amino-6-benzimidazolyl phenylketone; Aminobenzylpenicillin; 5-Amino-1-bis(dimethylamide) phosphoryl-3-phenyl-1,2,4-triazole; 2-Amino-5-bromo-6-phenyl-4 (1 h)-pyrimidinone; 4-Amino-2-(4-butanoylhexahydro-1 h-1,4-diazepin-1-yl)-6,7-dimethoxyquinazoline hydrochloride; 2-Amino-5-butylbenzimidazole; 5-Amino-1,6-dihydro-7h-v-triazolo (4,5-d) pyrimidin-7-one; 3-(2-aminoethyl)indol-5-ol; 3-(2-aminoethyl) indol-5-ol creatinine sulfate; trans-4-Aminoethylcyclohexane-1-carboxylic acid; Aminoglutethimide; 2-Amino-3-hydroxybenzoic acid; 8-Amino-7-hydroxy-3,6-napthalenedisulfonic acid, sodium salt;
4-Amino-n-(6-methoxy-3-pyridazinyl)-benzenesulfonamide; 3-Amino-4-methylbenzenesulfonylcyclohexylurea; 2-Amino-6-(1,-methyl-4,-nitro-5,-imidazolyl)mercaptopurine; 1-(4-Amino-2-methylpyrimidin-5-yl)methyl-3-(2-chloroethyl)-3-nitrosourea; 2-Amino-4-(methylsulfinyl)butyric acid; 5-Amino-2-napthalenesulfonic acid sodium salt; 6-Aminonicotinamide; 2-Amino-4-nitroaniline; 4-Amino-2-nitroaniline; Aminonucleoside puromycin; 2-Aminophenol; 3-Aminophenol; 4-Aminophenol; meta-Aminophenol, chlorinated; 7-(d-alpha-aminophenylacetamido) desacetoxycephalosporanic acid; 3-Aminopropionitrile; beta-Aminopropionitrile fumarate; Aminopropyl aminoethylthiophosphate; 3-(2-Aminopropyl)indole; Aminopteridine; 2-Aminopurine-6-thiol; Aminopyrine sodium sulfonate; Aminopyrine-barbital; 5-Amino-2-beta-d-ribofuranosyl-as-triazin-3-(2H)-one; 4-Amino-2,2,5,5-tetrakis(trifluoromethyl)-3-imidazoline; 2-Amino-1,3,4-thiadiazole; 2-Amino-1,3,4-thiadiazolehydrochloride; 2-Amino-1,3,4-thiadiazole-5-sulfonamide sodium salt; 1-Amino-2-(4-thiazolyl)-5-benzimidazolecarbamic acid isopropyl ester; Amitriptyline-n-oxide; Amitrole; Ammonium vanadate; Amosulalol hydrochloride; Amoxicillin trihydrate; dl-Amphetamine sulfate; Ampicillin trihydrate; Anrinone; Amsacrine lactate; Amygdalin; Anabasine; Anatoxin I; Androctonus amoreuxi venom; Androfluorene; Androfurazanol; Androstanazol; Androstenediol dipropionate; Androstenedione; Androstenolone; Androstestone-M; Angel dust; Angiotonin; Anguidin; Aniline violet; 6-(para-anilinosulfonyl)metanilamide; 2-Anthracenamine; Antibiotic BB-K8; Antibiotic BB-K8 sulfate; Antibiotic BL-640; Antibiotic MA 144A1; Antimony oxide; Apholate; 9-beta-d-Arabino furanosyl adenine; Arabinocytidine; Ara-C palmitate; Araten phosphate; Arathane; 1-Arginine monohydrochloride; Aristocort; Aristocort acetonide; Aristocort diacetate; Aristolic acid; Aristospan; Aromatol; Arotinoic acid; Arotinoic methanol; Arotinoid ethyl ester; Arsenic; ortho-Arsenic acid; Arsenic acid, disodium salt, heptahydrate; Arsenic acid, sodium salt; Arsenic trioxide; Asalin; 1-Ascorbic acid; 1-Asparaginase; Atrazine; Atromid S; Atropine; Atropine sulfate (2:1); Auranofin; Aureine; 1-Aurothio-d-glucopyranose; Ayush-47; Azabicyclane citrate; Azactam; Azacytidine; Azaserine; Azathioprine; Azelastine hydrochloride; 1-2-Azetidinecarboxylic acid; Azinphos methyl; Azo blue; Azo ethane; Azosemide; Azoxyethane; Azoxymethane; Baccidal; Bacmecillinam; Bal; Barbital sodium; Barium ferrite; Barium fluoride; Bayer 205; Baythion; Befunolol hydrochloride; Bendacort; Bendadryl hydrochloride; Benedectin; Benomyl; Benzarone; d-Benzedrine sulfate; Benzenamine hydrochloride; Benzene; Benzene hexachloride-g-isomer; 1-Benzhydryl-4-(2-(2-hydroxyethoxy)ethyl)piperazine; Benzidamine hydrochloride; 2-Benzimidazolecarbamic acid; 1-(2-Benzimidazolyl)-3-methylurea; 1,2-Benzisothiazol-3(2H)-one-1,1-dioxide; 1,2-Benzisoxazole-3-methanesulfonamide; Benzo (alpha) pyrene; Benzo (e) pyrene; Benzoctamine hydrochloride; para-Benzoquinone monoamine; Benzothiazole disulfide; 2-Benzothiazolethiol; 2-Benzothiazolyl-N-morpholinosulfide; 2-(meta-Benzoylphenyl)propionic acid; 2-Benzylbenzimidazole; Benzyl chloride; Benzyl penicillinic acid sodium salt; Beryllium chloride; Beryllium oxide; Bestrabucil; Betamethasone; Betamethasone; acetate and betamethasone phosphate; Betamethasone benzoate; Betamethasone dipropionate; Betamethasone disodium phosphate; Betel nut; Betnelan phosphate; BHT (food grade); Bindon ethyl ether; Binoside; 4-Biphenylacetic acid; 2-Biphenylol; 2-Biphenylol, sodium salt; 3-(4-Biphenylylcarbonyl)propionic acid; 2,2-Bipyridine; Bis(para-acetoxyphenyl)-2-methylcylcophexylidenemethane; 4,4-Bis(1-amino-8-hydroxy-2,4-disulfo-7-napthylazo)-3,3,-bitolyl,tetrasodium salt; 1,4-Bis(3-bromopropionyl)-piperazine; 1,3-Bis(carbamoylthio)-2-(N,N-dimethylamino)propane hydrochloride; trans-N,N,-Bis(2-chlorobenzyl)-1,4 cyclohexanebis(methylamine) dihydrochloride; Bis(2-chloroethyl)amine hydrochloride; 4,-(Bis(2-chloroethyl)amino)acetanilide; 4,-(Bis(2-chloroethyl)amino)-2-fluoro acetanilide; dl-3-(para-(Bis(2-chloroethyl)amino)phenyl)alanine; Bis(beta-chloroethyl)methylamine; Bis(2-chloroethyl)methylamine hydrochloride; Bis(2-chloroethyl)sulfide; N,N,-Bis(2-chloroethyl)-N-nitrosourea; N,N,-Bis(2-chloroethyl)-para-phenylenediamine; Bis(para-chlorophenyl)acetic acid; 2,2-Bis(ortho, para-chlorophenyl)-1,1,1-trichloroethane; 1,1-Bis(para-chlorophenyl)-2,2,2-trichloroethanol; Bis(beta-cyanoetyl)amine; Bis(dichloroacetyl)-1,8-diaminooctane; 3,5-Bis-dimethylamino-1,2,4-dithiazolium chloride; Bis(dimethyldithiocarbamato) zinc; (((3,5-Bis(1,1-dimethylethyl)-4-hydroxyphenyl)methyl)thio)acetic acid 2-ethylhexyl ester; Bis(dimethylthiocarbamoyl)sulfate; 2,4-Bis(ethylamino)-6-chloro-s-triazine; Bis(ethylmercuri) phosphate; Bis-HM-A-TDA; Bishydroxycoumarin; Bis(4-hydroxy-3-coumarin) acetic acid ethyl ester; 1,4-Bis((2-((2-hydroxyethyl)amino)ethyl)amino)-9,10-athracenedione diacetate; Bis isooctyloxycarbonylmethylthio)dioctyl stannane; Bis(2-methoxy ethyl)ether; Bisphenol A; 1,4-Bis(phenyl amino)benzene; Bis(tributyl tin) oxide; 2-(3,5-Bis(trifluoromethyl)phenyl)-N-methyl-hydrazinecarbothioamide (9CI); Bladex; Bleomycin sulfate; Bomt; Bracken fern, dried; Bradykinin; Bredinin; Bremfol; Bromacil; Bromazepam; Bromocriptine; Bromocriptine mesilate; 5-Bromo-2,-deoxyuridine; 2-Bromo-d-lysergic acid diethylamide; 6-Bromo-1,2-napththoquinone; Bromoperidol; Bromophenophos; 4-Bromophenyl chloromethyl sulfone; Buclizine dihydrochloride; Budesonide; Bunitrolol hydrochloride; Buprenorphine hydrochloride; 1,3-Butadiene; Butamirate citrate; 1,4-Butanediamine; 1,4-Butanediol dimethyl sulfonate; 4-Butanolide; Butobarbital; Butoctamide semisuccinate; Butorphanol tartrate; Butoxybenzyl hyoscyamine bromide; 2-Butoxyethanol; para-Butoxyphenylacetohydroxamic acid; Butriptyline; Bromoperidol; Bromophenophos; 4-Bromophenyl chloromethyl sulfone; Buclizine dihydrochloride; Budesonide; Bunitrolol hydrochloride; Buprenorphine hydrochloride; 1,3-Butadiene; Butamirate citrate; 1,4-Butanediamine; 1,4-Butanediol dimethyl sulfonate; 4-Butanolide; Butobarbital; Butoctamide semisuccinate; Butorphanol tartrate; Butoxybenzyl hyoscyamine bromide; 2-Butoxyethanol; para-Butoxyphenylacetohydroxamic acid; Butriptyline; n-Butyl acetate; n-Butyl alcohol; sec-Butyl alcohol; tert-Butyl alcohol; alpha,-((tert-Butyl amino)methyl)-4-hydroxy-meta-xylene-alpha,alpha-diol; Butyl carbamate; Butyl carbobutoxymethyl phthalate; Butyl dichlorophenoxyacetate; Butyl ethyl acetic acid; Butyl flufenamate; n-Butyl glycidyl ether; n-Butyl mercaptan; n-Butyl-3,ortho-acetyl-12-b-13-alpha-dihydrojervine; 1-(tert-Butylamino)-3-(2-chloro-5-methylphenoxy)-2-propanol hydrochloride; alpha-Butylbenzenemethanol; 5-Butyl-2-benzimidazolecarbamic acid methyl ester; 5-Butyl-1-cylcohexylbarbituric acid; 2-sec-Butyl-4,6-dinitrophenol; 4-Butyl-1,2-diphenyl-3,5-dioxo pyrazolidine; n-Butyl-N-nitroso-1-butamine; N-Butyl-N-nitroso ethyl carbamate; n-Butylnitrosourea; 1-Butyl-2′,6′-pipecoloxylidide; 1-Butyl-3-sulfanilyl urea;
1-Butyl-3-(para-tolyl sulfonyl)urea; 1-Butyl-3-(para-tolylsulfonyl)urea, sodium salt; Butyl-2,4,5-trichlorophenoxyacetate; 1-Butyryl-4-(phenylallyl)piperazine hydrochloride; Buzepide methiodide; Cadmium; Cadmium (II) acetate; Cadmium chloride; Cadmium chloride, dihydrate; Cadmium compounds; Cadmium oxide; Cadmium sulfate (1:1); Cadmium sulfate (1:1) hydrate (3:8); Cadralazine; Caffeic acid; Caffeine; Calcium EbrA complex; Calcium fluoride; Calcium phosphonomycin hydrate; Calcium trisodium diethylene triamine pentaacetate; Calcium valproate; Calcium-N2-ethylhexyl-beta-oxybutyramide semisuccinate; Cambendazole; Camphorated oil; Candida albicans glycoproteins; Cannabidiol; Cannabinol; Cannabis; Cap; Caprolactam; Captafol; Captan; Carbamates; Carbaryl; Carbendazim and sodium nitrite (5:1); Carbidopa; Carbinilic acid isopropyl ester; Carbofuran; Carbon dioxide; Carbon disulfide; Carbon monoxide; Carbon tetrachloride; Carboprost tromethamine; Cargutocin; Carmetizide; Carmofur; 1-Carnitine hydrochloride; Carnosine; Carzinophilin; Cassaya, manihot utilissima; Catatoxic steroid No. 1; d-Catechol; CAZ pentahydrate; Cefamandole sodium; Cefotaxime sodium; Cefazedone; Cefazolin sodium salt; Cefinetazole; Cefinetazole sodium; Cefroxadin; Cefuroxim; Celestan-depot; Cellryl; Cellulose acetate monophthalate; Centbucridine hydrochloride; Centchroman; Cephalothin; Cervagem; Cesium arsenate; Cethylamine hydrofluoride; alpha-Chaconine; Chenodeoxycholic acid; Chlodithane; Chlorambucil; Chloramphenicol; Chloramphenicol monosuccinate sodium salt; Chloramphenicol palmitate; Chlorcyclizine hydrochloride; Chlorcyclizine hydrochloride A; hlorcyclohexamide; Chlordane; Chlorimipramine; Chlorinated camphene; Chlorinated dibenzo dioxins; Chlorisopropamide;
Chlormadinon; para-Chloro dimethylaminoazobenzene; 2-Chloroadenosine; 1-(3-Chloroallyl)-3,5,7-triaza-1-azoniaadamantane chloride; 3-Chloro-4-aminoaniline; 1-((para-(2-(Chloro-ortho-anisamido)ethyl)phenyl)sulfonyl)-3-cylcohexyl urea; Chlorobenzene; ortho-Chlorobenzylidene malononitrile; 1-para-Chlorobenzyl-1H-indazole-3-carboxylic acid; 7-Chloro-5-(ortho-chlorophenyl)-1,3-dihydro-3-hydroxy-2H-1,4-benzodiazepin-2-one; Chlorocylcine; 6-Chloro-5-Cyclohexyl-1-indancarboxylic acid; 6-Chloro-5-(2,3-dichlorophenoxy)-2-methylthio-benzimidazole; 5-Chloro-2-(2-(diethylamino)ethoxy)benzanilide; 7-Chloro-1,3-dihydro-5-phenyl, 2H-1,4-benzodiazepin-2-one; Chloroethyl mercury; 1-(2-Chloroethyl)-3-cylcohexyl-1-nitrosourea; 1-Chloro-3-ethyl-1-penten-4-YN-3-OL; Chloroform; 4-Chloro-N-furfuryl-5-sulfamoylanthranilic acid; Chlorogenic acid; endo-4-Chloro-N-(hexahydro-4,7-methanoisoindol-2-YL)-3-sulfamoylbenzamide; (−)—N-((5-Chloro-8-hydroxy-3-methyl-1-OXO-7-isochromanyl)carbonyl)-3-phenylalanine; 5-Chloro-7-iodo-8-quinolinol; (4-Chloro-2-methylphenoxy)acetic acid; 2-(4-Chloro-2-methylphenoxy)propanoic acid (R) (9CI); 4-Chloro-2-methylphenoxy-alpha-propionic acid; 7-Chloro-1-methyl-5-phenyl-1H-1,5-benzodiazepine-2,4(3H,5H)-dione; 2-Chloro-11-(4-methylpiperazino) dibenzo (b,f) (1,4) thiazepine; 4-((5-Chloro-2-OXO-3(2H)-benzothiazolyl)acetyl)-1-piperazineethanol; 4-(3-(2-Chlorophenothiazin-10-YL)propyl)-1-piperazineethanol; 4-Chlorophenylalanine; 1-(para-Chloro-alpha-phenylbenzyl)-4-(2-((2-hydroxyethoxy)ethyl)piperazine); 1-(meta-Chlorophenyl)-3-N,N-dimethylcarbamoyl-5-methoxypyrazole; 3-(para-Chlorophenyl)-1,1,dimethylurea; 5,(2-Chlorophenyl)-7-ethyl-1-methyl-1,3-dihydro-2H-thieno (2,3-e) (1,4) diazepin-2-one; N-3-Chlorophenylisopropylcarbamate; 3-(4-Chlorophenyl)-1-methoxy-1-methylurea; 2-(ortho-Chlorophenyl)-2-(methylamino)cyclohexanone hydrochloride; 3-(para-Chlorophenyl)-1-methyl-1-(1-methyl-2-propynyl)urea; 4-(para-Chlorophenyl)-2-phenyl-5-thiazoleacetic acid; 1-(para-Chlorophenylsulfonyl)-3-propylurea; para-Chlorophenyl-2,4,5-trichlorophenyl sulfone; 4-Chlorophenyl-2,4,5-trichlorophenylazosulfide mixed with 1,1-bis(4-chlorophenyl)ethanol; Chloropromazine; Chloropromazine hydrochloride; Chloroquine; Chloroquine diphosphate; N-(3-Chloro-ortho-tolyl)anthranilic acid; 2-((4-Chloro-ortho-tolyl)oxy)propionic acid potassium salt; Chloro(triethylphosphine)gold; Chlorovinylarsine dichloride; 4-Chloro-3,5-xylenol;
Chlorphentermine; g-(4-(para-Chlorphenyl)-4-hydropiperidino)-para-fluorbutyrophenone; Cholecalciferol; Cholesterol; Cholestyramine; Chorionic gonadotropin; Chromium chloride; Chromium (VI) oxide (1:3); Chromium trichloride hexahydrate; Chromomycin A3; C.I. 45405; C.I. Direct blue 1, tetrasodium salt; C.I. Direct blue 6, tetrasodium salt; C.I. Direct blue 14, tetrasodium salt; C.I. Direct blue 15, tetrasodium salt; Cilostazol; Cinoxacin; Citreoviridin; Citrinin; Citrus hystrix DC., fruit peel extract; Clavacin; Clindamycin-2-palmitate monohydrochloride; Clindamycin-2-phosphate; Cloazepam; Clobetasone butyrate; Cloconazole hydrochloride; Clofedanol hydrochloride; Clofexamide phenylbutazone; Clomiphene; racemic-Clomiphene citrate; trans-Clomiphene citrate; Clonidine hydrochloride; Clonixic acid; Cloxazolazepam; Clozapine; Coagulase; Cobalt (III) acetylacetonate; Cobalt (II) chloride; Corn oil; Corticosterone; Corticosterone acetate; Cortisol; Cortisone; Cortisone-21-acetate; Cottonseed oil (unhydrogenated); Coumarin; Cravetin; meta-Cresol; Cumoesterol; S-1-Cyano-2-hydroxy-3-butene; Cyanotrimethylandrostenolone; Cycasin; Cyclocytidine hydrochloride; Cycloguanyl; Cyclohexanamine hydrochloride; Cycloheximide; Cyclohexylamine; Cyclohexylamine sulfate; 2-(Cyclohexylamino)ethanol;
N-Cyclohexyl-2-benzothiazolesulfenamide; 4-(4-Cyclohexyl-3-chlorophenyl)-4-oxobutyric acid; 1-Cyclohexyl-3-para-tolysulfonylurea; Cyclonite; Cyclopamine; Cyclophosphamide hydrate; Cyclophosphoramide; alpha-Cyclopiazonic acid; 5-(Cyclopropylcarbonyl)-2-benzimidazolecarbamic acid methyl ester; Cyprosterone acetate; Cysteine-germanic acid; Cytochalasin B; Cytochalasin E; Cytostasan; Cytoxal alcohol; Cytoxyl amine; Demeton-O+Demeton-S; Demeton-O-methyl; Demetrin; Denopamine; 11-Deoxo-12-beta, 13-alpha-dihydro-11-alpha-hydroxyj ervine; 11-Deoxoj ervine-4-EN-3-one; 2,-Deoxy-5-fluorouridine; 2-Deoxyglucose; 2,-Deoxy-5-iodouridine; 4-Deoxypyridoxol hydrochloride; Dephosphate bromofenofos; Depofemin; Depo-medrate; N-Desacetylthiocolchicine; Desoxymetasone; 2-Desoxyphenobarbital; Detergents, Liquid containing AES; Detergents, Liquid containing LAS; Dexamethasone acetate; Dexamethasone 17,21-dipropionate; Dexamethasone palmitate; Dextran 1; Dextran 70; Dextropropoxyphene napsy; alpha-DFMO; Diabenor; Diacetylmorphine hydrochloride; Dialifor; Diamicron; 2,4-Diamino-6-methyl-5-phenylpyrimidine; 2,4-Diamino-5-phenyl-6-ethylpyrimidine; 2,4-Diamino-5-phenyl-6-propylpyrimidine; 2,4-Diamino-5-phenylpyrimidine; 2,5-Diaminotoluene dihydrochloride; Diazepam; Diazinon;
6-Diazo-5-oxonorleucine; Diazoxide; Dibekacin; 5H-Dibenz(b,f) azepine-5-carboxamide; 5H-Dibenz(b,f) azepine, 3-chloro-5-(3-(4-carbamoyl-4-piperidinopiperine; Dibenz(b,f) (1,4) oxazepine; Dibenzacepin; Dibenzyline hydrochloride; 1,2-Dibromo-3-chloropropane; 3,5-Dibromo-4-hydroxyphenyl-2-ethyl-3-benzofuranyl ketone; Dibromomaleinimide; 1,6-Dibromomannitol; Dibutyl phthalate; N,N-Di-n-butylformamide; Dibutyryl cyclic amp; Dicarbadodecaboranylmethylethyl sulfide; Dicarbadodecaboranylmethylpropyl sulfide; 1-(2,4-Dichlorbenzyl)indazole-3-carboxylic acid; Dichloroacetonitrile; (ortho-((2,6-Dichloroanilino)phenyl)acetic acid sodium salt; ortho-Dichlorobenzene; para-Dichlorobenzene; 4,5-Dichloro-meta-benzenedisulfonamide; 2,2,-Dichlorobiphenyl; Dichloro-1,3-butadiene; 1,4-Dichloro-2-butene; 2,2-Dichloro-1,1-difluorethyl methyl ether; 5,5-Dichloro-2,2,-dihydroxy-3,3,-dinitrobiplienyl; 1,1-Dichloroethane; 2,3-Dichloro-N-ethylmaleinimide; Dichloromaleimide; Dichloro-N-methylmaleimide; 2,4-Dichloro-4,-nitrodiphenyl ether; 2,4-Dichlorophenol; (2,4-Dichlorophenoxy)acetic acid butoxyethyl ester; (2,4-Dichlorophenoxy)acetic acid dimethylamine; 4-(2,4-Dichlorophenoxy)butyric acid; 2-(2,4-Dichlorophenoxy)propionic acid; (+)-2-(2,4-Dichlorophenoxy)propionic acid; 3,4-Dichlorophenoxyacetic acid; 2,4-Dichlorophenoxyacetic acid propylene glycol butyl ether ester; 2-(2,6-Dichlorophenylamino)-2-imidazoline; 3,6-Dichloro-2-pyridinecarboxylic acid; Dichlorvos; Dicyclohexyl adipate; Dicyclohexyl-18-crown-6; Dicyclopentadienyldichlorotitanium; 7,8-Didehydroretinoic acid; Dieldrin; Diethyl carbitol; Diethyl carbonate; Diethyl mercury; Diethyl phthalate; Diethyl sulfate; 2-(Diethyl)amino)-2′,6′-acetoxylidide; 2-Diethylamino-2′,6′-acetoxylidide hydrochloride; ortho-(Diethylaminoethoxy)benzanilide; 2-(2-(Diethylamino)ethoxy)-5-bromobenzanilide; 2-(2-(Diethylamino)ethoxy)-2,-chloro-benzanilide; 2-(2-(Diethylamino)ethoxy)-3,-chloro-benzanilide; 2-(2-(Diethylamino)ethoxy)-3,-chloro-methylbenzanilide; (para-2-Diethylaminoethoxyphenyl)-1-phenyl-2-para-anisylethanol; 1-(2-(Diethylamino)ethyl)reserpine; 7-Diethylamino-5-methyl-s-triazolo(1,5-alpha) pyrimidine; N,N-Diethylbenzenesulfonamide; Diethylcarbamazine; Diethylcarbamazine acid citrate; Diethyldiphenyl dichloroethane; Diethylene glycol; Diethylene glycol monomethyl ether; 1,2-Diethylhydrazine; 1,2-Diethylhydrazine dihydrochloride; N,N-Diethyllsergamide; N,N-Diethyl-4-methyl-3-oxo-5-alpha-4-azaandrostane-17-beta-carboxamide; 3,3-Diethyl-1-(meta-pyridyl)triazene; a,a-Diethyl-(E)-4,4,-stilbenediol bis(dihydrogen phosphate); a,a-Diethyl-4,4,-stilbenediol disodium salt; Diethylstilbesterol; Diethylstilbestrol dipalmitate; Diethylstilbestrol dipropionate; Diflorasone diacetate; Diflucortolone valerate; dl-alpha-Difluoromethylomithine; 5-(2,4-Difluorophenyl)salicylic acid; Difluprednate; Digoxin; Dihydantoin; Dihydrocodeinone bitartrate; Dihydrodiethylstilbestrol; 3,4-Dihydro-6-(4-(3,4-dimethoxybenzoyl)-1-piperazinyl)-2(1H)— quinolinone; 5,6-Dihydro-N-(3-(dimethylamino)propyl)-11H-dibenz(b,e)azepine; 10,11-Dihydro-5-(3-(dimethylamino)propyl)-5H-dibenz(b,f)azepine hydrochloride; 5,6-Dihydro-para-dithiin-2,3-dicarboximide; 12,b,13,alpha-Dihydrojervine; 10,11-Dihydro-5-(3-methylamino)propyl)-5H-dibenz(b,f)azepine hydrochloride; 1,7-Dihydro-6H-purin-6-one; 7,8-Dihydroretinoic acid; Dihydrostreptomycin; 4-Dihydrotestosterone; 3-alpha,17-beta-Dihydroxy-5-alpha-androstane; 3-alpha,7-beta-Dihydroxy-6-beta-cholan-24-OIC acid;
1 alpha,25-Dihydroxycholecalciferol; 3,4-Dihydroxy-alpha-((isopropylamino)methyl)benzyl alcohol; 1-Dihydroxyphenyl-1-alanine; 1-(−)-3-(3,4-Dihydroxyphenyl)-2-methylanine; 17R,21-alpha-Dihydroxy-4-propylajmalanium hydrogen tartrate; DI(2-Hydroxy-n-propyl)amine; Diisobutyl adipate; Diisobutyl phthalate; alpha-(2-(Diisopropylamino)ethyl)-alpha-phenyl-2-pyridineacetamide; Dilantin; Dilaudid; Diltiazem hydrochloride; Dimatif; Dimethoxy ethyl phthalate; 1,2-Dimethoxyethane; 3,6-Dimethoxy-4-sulfanilamidopyridazine; Dimethyl adipate; O,O-Dimethyl methylcarbamoylmethyl phosphordithioate; Dimethyl phthalate; Dimethyl sulfate; Dimethyl sulfoxide; O,S-Dimethyl phosphoramidothioate; N,N-Dimethylacetamide; O,O-Dimethyl-S-(2-(acetylamino)ethyl)dithiophosphate; 4-(Dimethylamine)-3,5-XYLYL-N-methylcarbamate; Dimethylaminoantipyrine; 4-Dimethylaminoazobenzene; para-Dimethylaminobenzenediazosodium sulphonate; 5-(3-(Dimethylamino)propyl)-2-hydroxy-10,11-dihydro-5H-dibenz(b,f)azephine; 11-(3-Dimethylaminopropylidene-6,11-dihydrodibenzo(b,e)thiepine hydrochloride; 10-(2-(Dimethylamino)propyl)phenothiazine; Dimethylbenzanthracene; 1,1-Dimethylbiguanide; 1-(2-(1,3-Dimethyl-2-butenylidene)hydrazino)phthalazine; Dimethyldicetylammonium chloride; 9,9-Dimethyl-10-dimethylaminopropylacridan hydrogen tartrate; 6-alpha,21-Dimethylethisterone; N-(5-(((1,1-Dimethylethyl)amino)sulfonyl)-1,3,4-thiadiazol-2-YL)acetamide monsodium salt; N,N-Dimethyl-para((para-fluorophenyl)azo)aniline; Dimethylformamide; 1,1-Dimethylhydrazine; 1,2-Dimethylhydrazine; 2,6-Dimethylhydroquinone; Dimethylimipramine; 1,3-Dimethylisothiourea; 1,3-Dimethylnitrosourea; 3,3-Dimethyl-1-phenyltriazene; Dimethylthiomethylphosphate; N,N-Dimethyl-4-(para-tolylazo)aniline; 5-(3,3-Dimethyl-1-triazeno)imidazole-4-carboxamide citrate; 2,6-Dimethyl-4-tridecylmorpholine; 1,3-Dimethylurea; 2,4-Dinitroaniline; 4,6-Dinitro-ortho-cresol ammonium salt; 2,6-Dinitro-N,N-dipropyl-4-(trifluoromethyl)benzenamine; 2,4-Dinitrophenol; 2,4-Dinitrophenol sodium salt; Dinitrosopiperazine; 2,4-Dinitrotoluene; 2,6-Dinitrotoluene; Dinoprost methyl ester; Dinoprostone; n-Dioctyl phthalate; Dioxane; meta-Dioxane-4,4-dimethyl; 1,4-Di-N-oxide of dihydroxymethylquinoxaline; 1,3-Dioxolane-4-methanol; 3-(2-(1,3-Dioxo-2-methylindanyl)) glutarimide; 3-(2-(1,3-Dioxo-2-phenyl-4,5,6,7-tetrahydro-4,7-dithiaindanyl)) glutarimide; 2-(2,6-Dioxopiperiden-3YL)phthalimide; N-(2,6-Dioxo-3-piperidyl)phthalimidine; 1,3-Dioxo-2-(3-pyridylmethylene)indan; Diphenylamine; Diphenylguanidine; Diphenylhydantoin and Phenobarbital; 3-(3,3-Diphenylpropylamino)propyl-3′,4′,5′-trimethoxybenzoate hydrochloride; Dipropyl adipate; Diquat; DI-sec-octyl phthalate; Disodium ethylene-1,2-bisidithiocarbamate; Disodium etidronate; Disodium inosinate; Disodium methanearsenate; Disodium molybdate dehydrate; Disodium phosphonomycin; Disodium selenate; Disulfuram; Dithane M-45; 2,2-Dithiobis(pyridine-1-oxide)magnesium sulfate trihydrate; 2,2-Dithiodipyridine-1,1,-dioxide; Diuron; alpha-DFMO; Dobutamine hydrochloride; Domperidone; Dopamine; Dopamine hydrochloride; Doriden; Doxifluridine; Doxycycline; 1-Dromoran tartrate; Duazomycin; Durabolin; Duricef; Dydrogesterone; Dye C; Econazole nitrate; Eflomithine hydrochloride; Elasiomycin; Elavil; Elavil hydrochloride; Elymoclavine; EM 255; Emoquil; Emorfazone; Enalapril maleate; Enavid; Endosulfan; Endrin; Enflurane; Enoxacin; Epe; Ephedrine; Epichlorohydrin; Epidehydrocholesterin; 2-alpha,3-alpha-Epithio-5-alpha-androstan-17-beta-OL; 4,5-Epithiovaleronitrile; EPN; Epocelin; 1,2-Epoxyethylbenzene; Eraldin; Ergochrome AA (2,2)-5-beta,6-alpha, 10-beta-5′,6′-alpha, 1-,-beta; Ergocomine methanesulfonate (salt); Ergotamine tartrate; Ergoterm TGO; Erythromycin; Escherichia coli endotoxin; Escin; beta-Escin; Escin, sodium salt; Estradiol; Estradiol dipropionate; Estradiol polyester with phosphoric acid; Estradiol-17-valerate; Estradiol-3-benzoate; Estradiol-3-benzoate mixed with progesterone (1:14 moles); Estradiol-17-caprylate; Estramustin phosphate sodium; Estra-1,3,5(10)-triene-17-beta-diol-17-tetrahydropyranyl ether; Estriol; Estrone; Ethanolamine; Ethinamate; Ethinyl estradiol; Ethinyl estradiol and norethindrone acetate; 17-alpha-Ethinyl-5,10-estrenolone; dl-Ethionine; Ethisterone and diethylstilbestrol; 6-Ethoxy-2-benzothiazolesulfonamide; 2-Ethoxyethanol; 2-Ethoxyethyl acetate; Ethyl alcohol; Ethyl all-trans-9-(4-methoxy-2,3,6-trimethylphenyl)-3,7-dimethyl-2,4,6,8-nonatetraenoate; Ethyl apovincaminate; Ethyl benzene; Ethyl (2,4-dichlorophenoxy)acetate; Ethyl fluclozepate; Ethyl hexylene glycol; Ethyl mercury chloride; Ethyl methacrylate; Ethyl methanesulfonate; Ethyl methyl 1,4-dihydro-2,6-dimethyl-4-(meta-nitrophenyl)-3,5-pyridinedicarboxylate; Ethyl morphine hydrochloride dehydrate; Ethyl thiourea; alpha-((Ethylamino)methyl)-meta-hydroxybenzyl alcohol; 2-Ethylamino-1,3,4-thiadiazole; 1-Ethyl-1,4-dihydro-7-methyl-4-oxo-1,8-naphthyridine-3-carboxylic acid; Ethyl-5-dimethylaminoethyl methylphosphonothiolate; Ethyl-N,N-dimethyl carbamate; Ethylene bis(dithiocarbamato)) zinc; Ethylene chlorohydrin; 1,2-Ethylene dibromide; Ethylene dichloride; Ethylene glycol; Ethylene glycol diethyl ether; Ethylene glycol methyl ether; Ethylene oxide; Ethylenebis (dithiocarbamato) manganese and zinc acetate (50:1); Ethylenediamine hydrochloride; Ethylenediaminetetraacetic acid; Ethylenediaminetetraacetic acid, disodium salt; Ethyleneimine; Ethylestrenol; 2-Ethylhexanol; Ethyl-para-hydroxyphenyl ketone; Ethylmercuric phosphate; Ethyl-N-methyl carbamate; Ethyl-2-methyl-4-chlorophenoxyacetate; 5-Ethyl-N-methyl-5-phenylbarbituric acid; 2-Ethyl-2-methylsuccinimide; 1-Ethyl-4-(2-morpholinoethyl)-3,3-diphenyl-2-pyrrolidinone; N-Ethyl-N-nitrosobiuret; 1-Ethyl-1-nitrosourea; Ethylnorgestrienone; 17-Ethyl-19-nortestosterone; N-Ethyl-para-(phenylazo) aniline; 5-Ethyl-5-phenylbarbituric acid; 1-5-Ethyl-5-phenylhydantoin; 3-Ethyl-5-phenylhydantoin; 5-(2-Ethylphenyl)-3-(3-methoxyphenyl)-s-triazole; 2-Ethylthioisonicotinamide; Ethyltrichlorphon; Ethyl-3,7,11-trimethyldodeca-2,4-dienoate; Ethylurea and sodium nitrite (1:1); Ethylurea and sodium nitrite (2:1); Ethynodiol; Ethynylestradiol mixed with norethindrone; 2-alpha-Ethynyl-alpha-nor-17-alpha-pregn-20-YNE-2-beta,17-beta-diol; Etizolam; Etoperidone; ETP;
E. typhosa lipopolysaccharide; False hellebore; Famfos; Famotidine; FD&C red No. 2; FD&C yellow NO. 5; Feldene; Fencahlonine; Fenestrel; Fenoprofen calcium dehydrate; Fenoterol hydrobromide; Fenthion;Fenthiuram; Ferbam; Ferrous sulfate; Fertodur; Fiboran; Firemaster BP-6; Firemaster FF-1; Flavoxate hydrochloride; Flomoxef sodium; Floxapen sodium; Flubendazole; Flucortolone; Flunarizine dihydrochloride; Flunisolide; Flunitrazepam; Fluoracizine; N-Fluoren-2-YL acetamide; Fluorobutyrophenone; Fluorocortisone; 5-Fluoro-2,-deoxycytidine; 3-Fluoro-4-dimethylaminoazobenzene; Fluorohydroxyandrostenedione; 2-Fluoro-alpha-methyl-(1,1,-biphenyl)-4-acetic acid 1-(acetyloxy)ethyl ester; 4,-Fluoro-4-(4-methylpiperidino)butyrophenone hydrochloride; 3-Fluoro-4-phenylhydratropic acid; 5-Fluoro-1-(tetrahydrofuran-2-YL)uracil; Fluorouracil; Flutamide; Flutazolam; Flutoprazepam; Flutropium bromide hydrate; Folic acid; Fominoben hydrochloride; Fonazine mesylate; Formaldehyde; Formamide; Formhydroxamic acid; Formoterol fumarate dehydrate; N-Formyl-N-hydroxyglycine; N-Formyljervine; Forphenicinol; Fortimicin A; Fortimicin A sulfate; Fotrin; Fulvine; Fumidil; Furapyrimidone; Furazosin hydrochloride; 2-(2-Furyl)-3-(5-nitro-2-furyl)acrylamide; Fusarenone X; Fusaric acid calcium salt; Fusariotoxin T 2; Fusidine;
Fyrol FR 2; Gabexate mesylate; Galactose; Gastrozepin; Gentamycin; Gentamycin sulfate; Gentisic cid; Germanium dioxide; Gestoral; Gindarine hydrochloride; Glucagon; 2-(beta-d-Glucopyranosyloxy)isobutyronitrile; d-Glucose; Gludiase; Glutaraldehyde; Glutril; Glycidol; Glycinonitrile; Glycinonitrile hydrochloride; Glycol ethers; Glycyrrhizic acid, ammonium salt; Gold sodium thiomalate; Gonadotropin releasing hormone agonist; Gossypol acetic acid; Grisofulvin; Guanabenz acetate; Guanazodine; Guanfacine hydrochloride;Guanine-3-N-oxide; Guanosine; HBK; Haloanisone; Halofantrine hydrochloride; Haloperidol decanoate; Halopredone acetate; Halothane; Haloxazolam; HCDD; Heliotrine; Hematoidin; Heptamethylphenylcyclotetrasiloxane; Heptyl phthalate; Heroin; Hexabromonaphthalene; Hexachlorobenzene; 2,2′,4,4′,5′5′-Hexachloro-1,1,-biphenyl; 3,3′,4,4′,5,5′-Hexachlorobiphenyl; Hexachlorobutadiene; Hexachlorocyclopentadiene; 1,2,3,4,7,8-Hexachlorodibenzofuran; Hexachlorophene; 4,5,6,7,8,8-Hexachlor-D1,5-tetrahydro-4,7-methanoinden; 1-Hexadecanamine; Hexadecyltrimethylammonium bromide; Hexafluoroacetone; Hexafluoro acetone trihydrate; Hexamethonium bromide; Hexamethylmelamine; n-Hexane; 1,6-Hexanediamine; 2-Hexanone; Hexocyclium methylsulfate; Hexone; Hexoprenaline dihydrochloride; Hexoprenaline sulfate; n-Hexyl carborane; Histamethizine; Histamine diphosphate; Homofolate; Human immunoglobin COG-78; Hyaluronic acid, sodium salt; Hycanthone methanesulfonate; Hydantoin; Hydralazine; Hydralazine hydrochloride; Hydrazine; Hydrochlorbenzethylamine dimaleate; Hydrochloric acid; Hydrocortisone sodium succinate; Hydrocortisone-21-acetate; Hydrocortisone-17-butyrate; Hydrocortisone-17-butyrate-21-propionate; Hydrocortisone-21-phosphate; Hydrofluoric acid; 10-beta-Hydroperoxy-17-alpha-ethynyl-4-estren-17-beta-OL-3-one; Hydroquinone-beta-d-glucopyranoside; N-Hydroxy ethyl carbamate; 4,-Hydroxyacetanilide; N-Hydroxy-N-acetyl-2-aminofluorene; N-Hydroxyadenine; 6-N-Hydroxyadenosine; 3-alpha-Hydroxy-17-androston-one; 17-beta-Hydroxy-5-beta-androstan-3-one; 3-Hydroxybenzoic acid; para-Hydroxybenzoic acid ethyl ester; 5-(alpha-Hydroxybenzyl)-2-benzimidazolecarbamic acid methyl ester; 1-Hydroxycholecalciferol; Hydroxydimethylarsine oxide; Hydroxydimethylarsine oxide, sodium salt; 9-Hydroxyellipticine; 2-(2-Hydroxyethoxy)ethyl-N-(alpha,alpha,alpha-trifluoro-meta-tolyl)anthranilate; Hydroxyethyl starch; beta-Hydroxyethylcarbamate; 1-Hydroxyethylidene-1,1-diphosphonic acid; 17-beta-Hydroxy-7-alpha-methylandrost-5-ENE-3-one; 7-Hydroxymethyl-12-methylbenz(alpha)anthracene; 1-Hydroxymethyl-2-methylditmide-2-oxide; 5-Hydroxymethyl-4-methyluracil; 2-Hydroxymethylphenol; 5-(1-Hydroxy-2-((1-methyl-3-phenylpropyl)amino)ethyl)salicyclamide hydrochloride N-(Hydroxymethyl)phthalimide; 3-(1-Hydroxy-2-piperidinoethyl)-5-phenylisoxazole citrate; 2-Hydroxy-N-(3-(meta-(piperidinomethyl)phenoxy)propyl)acetamide acetate (ester hydrochloride); Hydroxyprogesterone caproate; beta-(N-(3-Hydroxy-4-pyridone))-alpha-aminopropionic acid; 4-Hydroxysalicylic acid; 5-Hydroxytetracycline; 5-Hydroxytetracycline hydrochloride; 17-beta-Hydroxy-4,4,17-alpha-trimethyl-androst-5-ENE(2,3-d) isoxazole; Hydroxytriphenylstannane; dl-Hydroxytryptophan; 5-Hydroxy-1-tryptophan; dl-Hydroxytryptophan; 5-Hydroxy-1-tryptophan; Hydroxyurea; 3-Hydroxyxanthine; Hydroxyzine pamoate; Hyoscine hydrobromide; Hypochlorous acid; Hypoglycine B; Ibuprofen piconol; Ifenprodil tartrate; IMET 3106; 4-hnidazo (1,2-alpha) pyridin-2-YL-alpha-methylbenzeneacetic acid; Imidazole mustard; 2-Imidazolidinethione; 2-Imidazolidinethione mixed with sodium nitrite; 2-Imino-5-phenyl-4-oxazolidinone; Improsulfan tosylate; Indacrinone; Indanazoline hydrochloride; 1,3-Indandione; Indapamide; Indeloxazine hydrochloride; Inderal; Indium; Indium nitrate; 1H-Indole-3-acetic acid; Indole-3-carbinol; Indomethacin; Inolin; Insulin; Insulin protamine zinc; locarmate meglumine; Iodoacetic acid; lopramine hydrochloride; Iotroxate meglumine; Ipratropium bromide; Iron-dextran complex; Iron nickel zinc oxide; Iron-poly(sorbitol-gluconic acid) complex; Iron-sorbitol; Isoamygdalin; Isoamyl 5,6-dihydro-7,8-dimethyl-4,5-dioxo-4H-pyrano (3,2-c) quinoline-2-carboxylate; Isobutyl methacrylate; para-Isobutylhydratropic acid; Isocarboxazid; Isodecyl methacrylate; Isodonazole nitrate; Isoflurane; Isonicotinic acid hydrazide; Isonicotinic acid-2-isopropylhydrazide; Isooctyl-2,4-dichlorophenoxyacetate; Isophosphamide; Isoprenaline hydrochloride; Isoprenyl chalcone; Isopropyl alcohol; Isopropyl-2,4-D ester; Isopropylidine azastreptonigrin; 4,4,-Isopropylidenediphenol, polymer with 1-chloro-2,3-epoxypropane; Isopropylmethanesulfonate; Isosafrole-n-octylsulfoxide; Isothiacyanic acid, ethylene ester; Isothiocyanic acid, phenyl ester; Isothiourea; Jervine; Jervine-3-acetate; Josamycin; Kanamycin; Kanamycin sulfate (1:1) salt; KAO 264; Karminomycin; Kepone; Kerlone; Ketamine; Ketoprofen sodium; Ketotifen fumarate; KF-868; Khat leaf extract; KM-1146; KPE; Lactose; Latamoxef sodium; Lead; Lead acetate (II), trihydrate; Lead chloride; Lead diacetate; Lead (II) nitrate (1:2); Lecithin iodide; Lenampicillin hydrochloride; Lendormin; Lente insulin; Lentinan; Leptophos; 1-Leucine; Leurocristine; Leurocristine sulfate (1:1); Levamisole hydrochloride; Levorin; Levothyroxine sodium; Librium; d-Limonene; Linear alkylbenzenesulfonate, sodium salt; Linoleic acid (oxidized); Liothyronine; Lipopolysaccharide, escherichia coli; Lipopolysaccharide, from B. Abortus Bang; Lithium carbonate (2:1); Lithium carmine; Lithium chloride; Lividomycin; Lobenzarit isodium; Locoweed; Lofetensin hydrochloride; Lucanthone metabolite; Luteinizing hormone antiserum; Luteinizing hormone-releasing hormone; Luteinizing hormone-releasing hormone, diacetate (salt); Luteinizing hormone-releasing hormone, diacetate, tetrahydrate; Lyndiol; Lysenyl hydrogen maleate; d-Lysergic acid diethylamide tartrate; Lysergide tartrate; Lysine; Mafenide acetate; Magnesium glutamate hydrobromide; Magnesium sulfate (1:1); Malathion; Maleimide; Malotilate; Maltose; Manganese (II) chloride (1:2); Manganese (II) ethylenebis(dithiocarbamate); Manganese (II) sulfate (1:1); Maprotiline hydrochloride; Marezine hydrochloride; Maytansine; Mazindol; Mec; Meclizine dihydrochloride; Meclizine hydrochloride; Medemycin; Medrogestone; Medroxyprogesterone; Medroxyprogesterone acetate; Medullin; Melengestrol acetate; Mentha arvensis, oil; Mepiprazole dihydrochloride; Mepyrapone; Mequitazine; 2-Mercapto-1-methylimidazole; 1-(d-3-Mercapto-2-methyl-1-oxopropyl)-1-proline (S,S); N-(2-Mercapto-2-methylpropanoyl)-1-cysteine; 6-Mercaptopurine monohydrate; 6-Mercaptopurine 3-N-oxide; Mercaptopurine ribonucleoside; d,3-Mercaptovaline; Mercuric acetate; Mercuric oxide; Mercury; Mercury (II) chloride; Mercury (II) iodide; Mercury methylchloride; Merthiolate sodium; Mervan ethanolamine salt; Mescaline; Mesoxalylurea monohydrate; Mestranol mixed with norethindrone; Metalutin; Metaproterenol sulfate; Methadone; Methadone hydrochloride; dl-Methadone hydrochloride; Methallyl-19-nortestosterone; Methaminodiazepoxide hydrochloride; 1-Methamphetamine hydrochloride; Methaqualone hydrochloride; Methedrine; di-Methionine; 1-Methionine; Methionine sulfoximine; Methofadin; Methophenazine difumarate; Methotrexate; Methotrexate sodium; Methoxyacetic acid; 3-Methoxycarbonylaminophenyl-N-3,-methylphenylcarbamate; Methoxychlor; 5-Methoxyindoleacetic acid; 4-(6-Methoxy-2-naphthyl)-2-butanone; (+)-2-(Methoxy-2-naphthyl)-propionic acid; 2-(3-Methoxyphenyl)-5,6-dihydro-s-triazolo (5,1-alpha) isoquinoline; 2-(para-(6-Methoxy-2-phenyl-3-indenyl)phenoxy)triethylamine hydrochloride; 2-(para-(para-Methoxy-alpha-phenylphenethyl)phenoxy)triethylamine hydrochloride; N1-(3-Methoxy-2-pyrazinyl)sulfanilamide; Methyl alcohol; Methyl azoxymethyl acetate; Methyl benzimidazole-2-YL carbamate; 2-Methyl butylacrylate; Methyl chloride; Methyl chloroform; Methyl(beta)-11-alpha-16-dihydroxy-16-methyl-9-oxoprost-13-EN-1-OATE; Methyl ethyl ketone; Methyl hydrazine; Methyl isocyanate; Methyl mesylate; Methyl methacrylate; Methyl(methylthio)mercury; Methyl parathion; Methyl pentachlorophenate; Methyl phenidyl acetate; Methyl salicylate; Methyl thiourea; Methyl urea and sodium nitrite; Methylacetamide; Methyl-5-benzoyl benzimidazole-2-carbamate; 1-Methyl-2-benzylhydrazine; 1-Methyl-5-chloroindoline methylbromide; Methylchlortetracycline; 3-Methylcholanthrene; N-Methyl-4-cyclochexene-1,2-dicarboximide; N-Methyl-N-desacetylcolchicine; N-Methyl-dibromomaleinimide; beta-Methyldigoxin; 17-alpha-Methyldihydrotestosterone; N-Methyl-3,6-dithia-3,4,5,6-tetrahydrophthalimide; Methylene chloride; Methylene dimethanesulfonate; N,N,-Methylenebis(2-amino-1,3,4-thiadiazole); 2-Methylenecyclopropanylalanine; Methylergonovine maleate; 3-(1-Methylethyl)-1H-2,1,3-benzothiazain-4(3H)-one-2,2-dioxide; 4-Methylethylenethiourea; 3-Methyl-5-ethyl-5-phenylhydantoin; 3-Methylethynylestradiol;
x-Methylfolic acid; N-Methylformamide; Methylhesperidin; (alpha-(2-Methylhydrazino)-para-toluoyl)urea, monohydrobromide; 4-Methyl-7-hydroxycoumarin; Methyl-ortho-(4-hydroxy-3-methoxycinnamoyl)reserpate; 2-Methyl-1,3-indandione; N-Methyljervine; N-Methyllorazepam;
Methylmercuric dicyandiamide; Methylmercuric phosphate; Methylmercury; Methylmercury hydroxide; 1-Methyl-6-(1-methylallyl)-2,5-dithiobiurea; d-3-Methyl-N-methylmorphinan phosphate; N-Methyl-alpha-methyl-alpha-phenylsuccinimide; 2-Methyl-1,4-naphthoquinone; 2-Methyl-5-nitroimidazole-1-ethanol; N-Methyl-N′-nitro-N-nitrosoguanidine; 4-(N-Methyl-N-nitrosamino)-1-(3-pyridyl)-1-butanone; N-Methyl-N-nitrosoaniline; N-Methyl-N-nitrosoethylcarbamate; N-Methyl-N-nitroso-1-propanamine; N-Methyl-N-nitrosourea; (3-Methyl-4-oxo-5-piperidino-2-thiazolidinylidene)acetic acid ethyl ester; 10-Methylphenothiazine-2-acetic acid; N-Methyl-para-(phenylazo) aniline; 3-Methyl-2-phenylmorpholine hydrochloride; N-Methyl-2-phenyl-succinimide; Methyl-4-phthalimido-dl-glutaramate; N-Methyl-2-phthalimidoglutarimide; N-Methylpyrrolidone; Methylsulfonyl chloramphenicol; 17-Methyltestosterone; N-Methyl-3,4,5,6-tetrahydrophthalimide; Methylthioinosine; 6-Methylthiouracil; 6-Methyluracil; Metiapine; Meticrane; Metoprine; Metoprolol tartrate; Metrizamide; Mexiletine hydrochloride; Mezinium methyl sulfate; Mezlocillin; Mibolerone; Miconazole nitrate; Micromycin; Midodrine; Mikelan; Miloxacin; Miltown; Mineral oil; Mineral oil, petroleum extracts, heavy naphthenic distillate solvent; Mirex; Mithramycin; MN-1695; Mobilat; Molybdenum; Monoethylhexyl phthalate; Monoethylphenyltriazene; 8-Monohydro mirex; Monosodium glutamate; Morphine ydrochloride; Morphine sulfate; Morphocycline; Moxestrol; Moxnidazole; Mucopolysaccharide, polysulfuric acid ester; Muldamine; Mycosporin; Nafoxidine hydrochloride; Naftidrofuryl oxalate; Naja igricollis venom; Naloxone hydrochloride; Naphthalene; beta-Naphthoflavone; 1-Naphthol; Navaron; Neem oil; Nembutal sodium; Neocarzinostatin; Neoprene; Neoproserine; Neosynephrine; Netilmicin sulfate; Nickel; Nickel carbonyl; Nickel compounds; Nickel subsulfide; Nickelous chloride; Nicotergoline; Nicotine; Nicotine tartrate (1:2); N-Nicotinoyltryptamide; Nipradilol; Nisentil; Nitric acid; Nitrilotriacetic acid trisodium salt monohydrate; Nitrobenzene; Nitrofurantoin; Nitrofurazone; 4-((5-Nitrofurfurylidene)amino)-3-methylthiomorpholine-1,1-dioxide; Nitrogen dioxide; Nitrogen oxide; Nitroglycerin; 1-(2-Nitroimidazol-1-YL-3-methoxypropan-2-OL; Nitromifene citrate; 2-Nitropropane; 4-Nitroquinoline-N-oxide; Nitroso compounds; N-Nitroso compounds; N-Nitrosobis(2-oxopropyl)amine; Nitrosocimetidine; N-Nitrosodiethylamine; N-Nitrosodimethylamine; N-Nitrosodi-N-propylamine; N-Nitroso-N-ethyl aniline; N-Nitroso-N-ethylurethan; N-Nitroso-N-ethylvinylamine; N-Nitrosohexahydroazepine; N-Nitrosoimidazolidinethione; N-Nitrosopiperidine; 1-(Nitrosopropylamino)-2-propanol; N-Nitroso-N-propylurea; Nizofenone fumarate; Norchlorcyclizine; Norchlorcyclizine hydrochloride; 1-Norepinephrine; 19-Norethisterone; Norethisterone enanthate; Norgestrel; 1-Norgestrel; 19-Norpregn-4-ENE-3,20-dione; 19-Nor-17-alpha-pregn-5(10)-EN-20-YNE-3-alpha,17-diol; 19-Nor-17-alpha-pregn-5(10)-EN-20-YNE-3-beta,17-diol; 19-Nor-17-alpha-pregn-4-EN-20-YN-17-OL; Novadex;
Nutmeg oil, east Indian; Nystatin; Ochratoxin; Ochratoxin A sodium salt; Octabromodiphenyl;
Octachlorodibenzodioxin; Octoclothepine; Ofloxacin; Oleamine; Oleylamine hydrofluoride; Oncodazole; Ophthazin; Orgoteins; Orphenadrine hydrochloride; Oxaprozin; Oxatimide; Oxazolazepam; Oxepinac; Oxfendazole; Oxibendazole; Oxiranecarboxylic acid, 3-(((3-methyl-1-(((3-methylbutyl)amino)carbonyl)-,ethyl ester, (2S-(2-alpha-3-beta)R*))); N-(2-Oxo-3,5,7-cylcoheptatrien-1-YL)aminooxoacetic acid ethyl ester; 2-(3-Oxo-1-indanylidene)-1,3-indandione; Oxolamine citrate;N-(2-Oxo-3-piperidyl)phthalimide; Oxybutynin chloride; Oxymorphinone hydrochloride; beta-Oxypropylpropylnitrosamine; Ozone; Padrin; Palm oil; Panoral; d-Pantethine; Pantocrin; Papain; Papaverine chlorohydrate; Paradione; Paramathasone acetate; Paraquat dichloride; Parathion; Paraxanthine; Pavisoid; PE-043; Penfluridol; Penicillic acid; Penitrem A; Pentachlorobenzene; 2,3,4,7,8-Pentachlorodibenzofuran; Pentachloronitrobenzene; Pentachlorophenol; Pentafluorophenyl chloride; Pentazocine hydrochloride; Pentostatin; Pentothal; Pentothal sodium; Pentoxyphylline; Perchloroethylene; Perdipine; Perfluorodecanoic acid; Periactin hydrochloride; Periactinol; Perphenazine hydrochloride; Pharmagel A; 1,10-Phenanthroline; Phenazin-5-oxide; Phenethyl alcohol; Phenfluoramine hydrochloride; Phenol; 4-Phenoxy-3-(pyrrolidinyl)-5-sulfamoylbenzoic acid; Phenyl salicylate; Phenylacetic acid; (Phenylacetyl)urea; 1-Phenylalanine; 17-beta-Phenylaminocarbonyloxyoestra-1,3,5(10)-triene-3-methyl ether; para-Phenylazo)aniline; 2-Phenyl-5-benzothiazoleacetic acid; 1-Phenyl-3,3-diethyltriazene; 2-Phenyl-5,5-dimethyl-tetrahydro-1,4-oxazine hydrochloride; 1-Phenyl-2-(1′,1′-diphenylpropyl-3′-amino)propane; 4-Phenyl-1,2-diphenyl-3,5-pyrazolidinedione; meta-Phenylenediamine; 2-Phenylethylhydrazine; Phenylmethylcylosiloxane, mixed copolymer; N-Phenylphthalimidine; Phenyl-2-pyridylmethyl-beta-N,N-dimethylaminoethyl ether succinate; 2-(Phenylsulfonylamino)-1,3,4-thiadiazole-5-sulfonamide; 1-Phenyl-2-thiourea; Phomopsin; Phorbol myristate acetate; Phosphonacetyl-1-aspartic acid; Phosphoramide mustard cyclohexylamine salt; Phthalazinol; Phthalic anhydride; Phthalimide; Phthalimidomethyl-O,O-dimethyl phosphorodithioate; N-Phthaloly-1-aspartic acid; N-Phthalylisoglutamine; Physostigmine sulfate; Phytohemagglutinin; Picloram; Pilocarpine monohydrochloride; Pimozide; 2,6-piperazinedione-4,4,-propylene dioxopiperazine; Piperidine; 3-Piperidine-1,1-diphenyl-propanol-(1) methanesulphonate; Piperin; Piperonyl butoxide; Pipethanate ethylbromide; Pipram; Pituitary growth hormone; Plafibride; cis-Platinous diammine dichloride; Platinum thymine blue; Podophyllin; Podophyllotoxin; Polybrominated biphenyls;
Polychlorinated biphenyl (Aroclor 1248); Polychlorinated biphenyl (Aroclor 1254); Polychlorinated biphenyl (Kanechlor 300); Polychlorinated biphenyl (Kanechlor 400); Polychlorinated biphenyl (Kanechlor 500); Polyoxyethylene sorbitan monolaurate; Potassium bichromate; Potassium canrenoate; Potassium chromate (VI); Potassium clavulanate; Potassium cyanide; Potassium fluoride; Potassium iodide; Potassium nitrate; Potassium:nitrite (1:1);
Potassium perchlorate; Potassium thiocyanate; Potato blossoms, glycoalkaloid extract; Potato, green parts; Pranoprofen; Prednisolone succinate; Prednisone 21-acetate; Predonin; 9-beta,10-alpha-Pregna-4,6-diene-3,20-dione and 17-alpha-hydroxypregn-4-ENE-3,2ortho-dione (9:10);
5-alpha-17-alpha-Pregna-2-EN-20-YN-17-OL, acetate; Premarin; Primaquine phosphate; Primobolan; Prinadol hydrobromide; Procarbazine; Procarbazine hydrochloride; Procaterol ydrochloride; Prochlorpromazine; Progesterone; Prolinomethyltetracycline; Promethazine hydrochloride; Propadrine hydrochloride; Propane sultone; 1,3-Propanediamine; 1,2-Propanediol; Propanidide; 3-Propanolamine; Proparthrin; Propazone; Propiononitrile; Propoxur; 2-Propoxyethyl acetate; d-Propoxyphene hydrochloride; Propyl carbamate; Propyl ellosolve; n-Propyl gallate; Propylene glycol diacetate; Propylene glycol monomethyl ether; Propylene oxide; 2-Propylpentanoic acid; 2-Propylpiperidine; 6-Propyl-2-thiouracil; Propylthiouracil and iodine; 2-Propylvaleramide; 2-Propylvaleric acid sodium salt; Prostaglandin A1; Prostaglandin E1; Prostaglandin E2 sodium salt; Prostaglandin F1-alpha; Prostaglandin F2-alpha; Prostaglandin F2-alpha-tham; Protizinic acid; Proxil; Pseudolaric acid A; Pseudolaric acid B; Purapuridine; Purine-6-thiol; Pyrantel pamoate; Pyrazine-2,3-dicarboxylic acid imide; Pyrazole; Pyrbuterol hydrochloride; Pyridinamine (9CI); 2,3-Pyridinedicarboximide; 3,4-Pyridinedicarboximide; 1-(Pyridyl-3)-3,3-dimethyl triazene; 1-Pyridyl-3-methyl-3-ethyltriazene; 5-(para-(2-Pyridylsulfamoyl)phenylazo)salicyclic acid; Pyrimidine-4,5-dicarboxylic acid imide; N1-2-Pyrimidinyl-sulfanilamide; Pyrogallol; Pyronaridine; N-(1-Pyrrolidinylmethyl)-tetracycline; Quaalude; Quercetin; Quinine; 2-Quinoline thioacetamide hydrochloride; Ralgro; Refosporen; Reptilase; Reserpine; Retinoid etretin; all-trans-Retinylidene methyl nitrone; Rhodamine 6G extra base; 2-beta-d-Ribofuranosyl-as-triazine-3,5(2H,4H)-dione; 1-beta-d-Ribofuranosyl-1,2,4-triazole-3-carboxamide; Ricin; Rifamycin AMP; Rifamycin SV;
Ripcord; Ritodrine hydrochloride; Rizaben; Robaveron; Ronnel; Rose bengal sodium; Rotenone;
Rowachol; Rowatin; R Salt; Rubratoxin B; Rythmodan; Salicyclaldehyde; Salicyclamide; Salicyclic acid; Salicyclic acid, compounded with morpholine (1:1); ortho-Salicylsalicylic acid; Salipran; Salmonella enteritidis endotoxin; Sarkomycin; SCH 20569; Scopolamine; Sefril; Selenium; Selenodiglutathione; Semicarbazide hydrochloride; Serum gonadotropin; Sfericase; Silicone 360; Sisomicin; S. Marcescens lipopolysaccharide; Smoke condensate, cigarette; Smokeless tobacco; Sodium para-aminosalicylate; Sodium arsenite; Sodium benzoate; Sodium bicarbonate; Sodium chloride; Sodium chlorite; Sodium chondroitin polysulfate; Sodium cobaltinitrite; Sodium colistinemethanesulfonate; Sodium cyanide; Sodium cyclamate; Sodium dehydroacetic acid; Sodium dichlorocyanurate; Sodium diethyldithiocarbamate; Sodium diphenyldiazo-bis(alpha-naphthylaminesulfonate); Sodium fluoride; Sodium (E)-3-(para-(1H-imidazol-1-methyl)phenyl)-2-propenoate; Sodium iodide; Sodium lauryl sulfate; Sodium luminal; Sodium nigericin; Sodium nitrite; Sodium nitrite and carbendazime (1:1); Sodium nitrite and 1-citrulline (1:2); Sodium nitrite and 1-(methylethyl)urea; Sodium nitroferricyanide;
Sodium pentachlorophenate; Sodium picosulfate; Sodium piperacillin; Sodium retinoate; Sodium saccharin; Sodium salicylate; Sodium selenite; Sodium selenite pentahydrate; Sodium sulfate (2:1); Sodium d-thyroxine; Sodium tolmetin dihydrate; Sodium-2,4-dichlorophenoxyacetate; (22s,25r)-5-alpha-Solanidan-3-beta-OL; Solanid-5-ENE-3-beta, 12-alpha-diol; (22s,25r)-Solanid-5-EN-3-beta-OL; Solanine; Solcoseryl; Spectogard; Spiclomazine hydrochloride; Spiramycin; Spiroperidol; SRC-II, heavy distillate; 1-ST-2121; Sterculia foetida oil; Steroids; Stimulexin; Streptomycin; Streptomycin and dihydrostreptomycin; Streptomycin sesquisulfate; Streptomycin sulphate; Streptonigran; Streptonigrin methyl ester; Streptozoticin;
STS 557; Styrene; Subtigen; Succinic anhydride; Succinonitrile; Sucrose; Sulfadiazine silver salt; Sulfadimethoxypyrimidine; Sulfadimethyldiazine; Sulfamonomethoxin; Sulfamoxole-trimethoprim mixture; Sulfanilamide; 6-Sulfanilamido-2,4-dimethoxypyrimidine;
5-Sulfanilamido-3,4-dimethyl-isoxazole; Sulfanilylurea; N-Sulfanylacetamide; alpha-Sulfobenzylpenicillin disodium; Sulfur dioxide; Sulfuric acid; Suloctidyl; Sultopride hydrochloride; Supercortyl; Superprednol; Surgam; Surital sodium; Surmontil maleate; Suxibuzone; Sweet pea seeds; Sygethin; meta-Synephrine hydrochloride; Synephrine tartrate; Synsac; 2,4,5-T; T-1982; T-2588; Tagamet; Tarweed; TCDD; Tellurium; Tellurium dioxide; Temephos; Tenormin; Terbutaline sulphate; Terodiline hydrochloride; Testosterone; Testosterone heptanoate; Testosterone propionate; 1,1,3,3-Tetrabutylurea; 2,3,7,8-Tetrachlododibenzofuran; Tetrachloroacetone; 1,1,3,3-Tetrachloroacetone; 3,3′,4,4′-Tetrachloroazoxbenzene; 1,2,3,4-Tetrachlorobenzene; 3,3′,4,4′-Tetrachlorobiphenyl; 2,4,5,6-Tetrachlorophenol; Tetracycline; Tetracycline hydrochloride; Tetraethyl lead; 1-trans-D9-tetrahydrocannabinol; 2-(para-(1,2,3,4-Tetrahydro-2-(para-chlorophenyl)naphthyl)phenoxy)triethyl amine; 2,3,4,5-Tetrahydro-2,8-dimethyl-5-(2-(6-methyl-3-pyridyl)ethyl)-1H-pyrid 0-(4,3-beta) indole; Tetrahydro-3,5-dimethyl-4H,1,3,5-oxadiazine-4-thione; 5,6,7,8-Tetrahydrofolic acid; 2-(1,2,3,4-Tetrahydro-1-naphthylamino)-2-imidazoline hydrochloride; 4,-O-Tetrahydropyranyladriamycin hydrochloride; para-(1,1,3,3-Tetramethylbutyl)phenol, polymer with ethylene oxide and formaldehyde 2,2,9,9-Tetramethyl-1,10-decanediol; Tetramethyl lead; Tetramethylsuccinonitrile; Tetramethylthiourea; 1,1,3,3-Tetramethylurea; Tetranicotylfructose; Tetrapotassium hexacyanoferrate; Tetrasodium fosfestrol; Tetrazosin hydrochloride dihydrate; Thalidomide; Thallium acetate; Thallium chloride; Thallium compounds; Thallium sulfate; Thebaine hydrochloride; para-(2-Thenoyl)hydratropic acid; Theobromine; Theobromine sodium salicylate; Theophylline; 1-(Theophyllin-7-YL)ethyl-2-(2-(para-chlorophenoxy)-2-methylpropionate; Thiamine chloride; 2-(Thiazol-4-YL) benzimidazole; 2-(4-Thiazolyl)-5-benzimidazolecarbamic acid methyl ester; Thioacetamide; Thioinosine; Thiotriethylenephosphoramide; 2-Thiouracil; Thiram; Thymidine; Thyroid; 1-Thyroxin; Thyroxine; Tiapride hydrochloride; Ticarcillin sodium; Ticlodone; Timepidium bromide; Timiperone; Tinactin; Tindurin; Timidazole; Tinoridine hydrochloride; Tiquizium bromide; 2,4,5-T isooctyl ester; Titanium (wet powder); Tizanidine hydrochloride; Tobacco; Tobacco leaf, nicotiana glauca; Tobramycin; Todralazine hydrochloride hydrate; Togal; Tolmetine; Toluene; para-Toluenediamine sulfate; ortho-Toluidine; Tormosyl; 2,4,5-T propylene glycol butyl ether ester; Traxanox sodium pentahydrate; Triaminoguanidine nitrate; para,para,-Triazenylenedibenzenesulfonamide; Triazolam; Trichloroacetonitrile; 1,2,4-Trichlorobenzene;
   Trichloroethylene; 2,4,4,-Trichloro-2,-hydroxydiphenyl ether; (2,2,2-Trichloro-1-hydroxyethyl)dimethylphosphonate; N-(Trichloromethylthio)phthalimide; 4-(2,4,5-Trichlorophenoxy)butyric acid; alpha-(2,4,5-Trichlorophenoxy)propionic acid; Trichloropropionitrile; Triclopyr; Tricosanthin; Tridemorph; Tridiphane; Triethyl lead chloride; Triethylenetetramine; 2,2,2-Trifluoroethyl vinyl ether; 3,-Trifluoromethyl-4-dimethylaminoazobenzene; Trifluoromethylperazine; 2-(8,-Trifluoromethyl-4,-quinolylamino)benzoic acid, 2,3-dihydroxy propyl ester; Trifluperidol; Triglyme; Trimebutine maleate; (beta)-Trimethoquinol; Trimethoxazine; 5-(3,4,5-Trimethoxybenzyl)-2,4-diaminopyrimidine; Trimethyl lead chloride;
   Trimethyl phosphate; Trimethyl phosphate; 3,3,5-Trimethyl-2,4-diketooxazolidine; Trimethylenedimethanesulfonate; exo-Trimethylenenorbornane; 1,1,3-Trimethyl-3-nitrosourea;
   1,3,5-Trimethyl-2,4,6-tris(3,5-DI-tert-butyl-4-hydroxybenzyl)benzene; Triparanol; Tris; Tris (1-aziridinyl)-para-benzoquinone; Tris-(1-aziridinyl)phosphine oxide; Trisaziridinyltriazine; Tris(1-methylethylene)phosphoric triamide; Tritolyl phosphate; Tropacaine hydrochloride; 1-Tryptophan; TSH-releasing hormone; Tungsten; dl-meta-Tyrosine; 1-Tyrosine; Ubiquinone 10;
   Uracil; Uracil mixture with tegafur (4:1); Uranyl acetate dihydrate; Urapidil; Urbacide; Urbason soluble; Urethane; Urfamicin hydrochloride; Uridion; Urokinase; Valbazen; Valison; Vanadium pentoxide (dust); Vasodilan; Vasodilian; Vasodistal; Vasotonin; Venacil; Ventipulmin; Veratramine; Veratrine; Veratrylamine; Vincaleukoblastine; Vincaleukoblastine sulfate (1:1) salt); Vinyl chloride; Vinyl pivalate; Vinyl toluene; Vinylidene chloride; R-5-Vinyl-2-xazolidinethione; Viomycin; Vipera berus venom; Viriditoxin; Visken; Vistaril hydrochloride;
   Vitamin A; Vitamin A acetate; Vitamin A acid; 13-cis-Vitamin A acid; Vitamin A palmitate; Vitamin B7; Vitamin B12 complex; Vitamin B12, methyl; Vitamin D2; Vitamin K; Vitamin MK 4; Volidan; Vomitoxin; Wait's green mountain antihistamine; Warfarin; Warfarin sodium; White spirit; Xamoterolfumarate; Xanax; Xanthinol nicotinate; Xylene; meta-Xylene; ortho-Xylene;
   para-Xylene; Xylostatin; N-(2,3-Xylyl)anthranilic acid; Ytterbium chloride Zaroxolyn; Zearalenone; Zimelidine dihydrochloride; Zinc carbonate (1:1); Zinc chloride; Zinc (II) EbrA complex; Zinc oxide; Zinc (N,N,-propylene-1,2-bis(dithiocarbamate)); Zinc pyridine-2-thiol-1-oxide; Zinc sulfate; Zoapatle, crude leaf extract; Zoapatle, semi-purified leaf extract; Zotepine; Zygosporin A; Zyloprim.

1. A method of reducing the severity of a birth defect in a mammal, the method comprising exposing the mammal, in utero, to a herpes virus amplicon particle comprising a cis element-flanked transgene and a sequence encoding a transposase, wherein, upon expression, the transposase inserts the transgene into the genome of a cell within the mammal and the transgene expresses a polypeptide or RNA that compensates for a protein or gene defect that is causally associated with the birth defect.

2. The method of claim 1, wherein the mammal is a human.

3. The method of claim 1 or, wherein the protein that is causally associated with the birth defect is an enzyme or hormone.

4. The method of claim 3, wherein the enzyme is hexosaminidase A (Hex-A).

5. The method of claim 3, wherein the enzyme is phenylalanine hydroxylase.

6. The method of claim 1, wherein the sequence encoding the transposase is Sleeping Beauty or a biologically active variant or mutant thereof.

7. The method of claim 1, wherein the herpes virus amplicon particle is made by a helper virus-free method.

8. The method of claim 1, wherein the cell is a neuron.

9. The method of claim 1, wherein the RNA mediates RNAi and compensates for a protein by mitigating the expression or activity of the protein.

10. A method of determining whether a polypeptide or RNA compensates for a protein or gene defect that is causally associated with a birth defect, the method comprising:
(a) providing a cell of a mammal, wherein the cell exhibits an abnormality exhibited by cells affected by the birth defect;
(b) exposing the cell to a herpes virus comprising a modified artificial chromosome, wherein the cell is exposed to the herpes virus for a time and under conditions in which the herpes virus transduces the cell and a nucleic acid sequence carried by the artificial chromosome is expressed as an RNA or polypeptide within the cell; and
(c) determining whether the RNA or polypeptide favorably alters the abnormality and thereby compensates for a protein that is causally associated with a birth defect.

11. The method of claim 10, wherein the protein that is causally associated with the birth defect is an enzyme or hormone.

12. The method of claim 11, wherein the enzyme is hexosaminidase A (Hex-A).

13. The method of claim 11, wherein the enzyme is phenylalanine hydroxylase.

14. The method of claim 10, wherein the mammal is a human.

15. The method of claim 10, wherein the cell is a neuron.

16. The method of claim 10, wherein the cell is a cell in culture.

17. The method of claim 10, wherein the modified artificial chromosome comprises:
(a) a pair of cleavage sites that flank
(i) a packaging/cleavage site of a herpes virus;
(ii) an ori of a herpes virus;
(iii) a first antibiotic resistance gene; and, optionally
(iv) a sequence that encodes a detectable marker;
(b) the nucleic acid sequence; and, optionally
(c) a second antibiotic resistance gene.

18. The method of claim 10, wherein the herpes virus is a herpes simplex virus, varicella zoster virus, Epstein-Barr virus, or cytomegalovirus.

19. The method of claim 10, wherein the herpes simplex virus is a type 1 (HSV-1), type 2 (HSV-2), type 3 (HSV-3), type 4 (HSV-4), type 5 (HSV-5), type 6 (HSV-6), type 7 (HSV-7), or type 8 (HSV-8) herpes simplex virus.

20. The method of claim 10, wherein the RNA mediates RNAi and compensates for a protein by mitigating the expression or activity of the protein.

21. Use of a herpes virus comprising a modified artificial chromosome in the treatment of a birth defect, wherein the artificial chromosome comprises a nucleic acid sequence that, when expressed as an RNA or polypeptide within a cell, compensates for a protein that is causally associated with the birth defect.

22. Use of a herpes virus comprising a modified artificial chromosome in the preparation of a medicament for the treatment of a birth defect, wherein the artificial chromosome comprises a nucleic acid sequence that, when expressed as an RNA or polypeptide within a cell, compensates for a protein that is causally associated with the birth defect.
